(19) United States
(12) Patent Application Publication
(10) Pub. No.: US 2021/0237035 A1
(43) Pub. Date: Aug. 5, 2021

(54) HOLLOW CORE GRANULES, PRODUCTS INCORPORATING THE GRANULES, AND METHODS OF PREPARING THE GRANULES

(71) Applicant: Church & Dwight Co., Inc., Princeton, NJ (US)

(72) Inventors: Andre Vieira Gomes, Jackson, NJ (US); Adam E. Butera, Fairless Hills, PA (US); Petr A. Egoyants, Saint Petersburg (RU); Irina A. Chepurnaya, Saint Petersburg (RU); Mikhail P. Karushev, Saint Petersburg (RU); Vadim A. Bykov, Saint Petersburg (US); Ilya Ilyn, Wayland, MA (US)

(73) Assignee: Church & Dwight Co., Inc., Princeton, NJ (US)

(21) Appl. No.: 17/238,882

(22) Filed: Apr. 23, 2021

Related U.S. Application Data
(60) Provisional application No. 62/014,911, filed on Jun. 20, 2014.

(51) Int. Cl. B01J 20/28; C11D 11/00; C11D 17/00; C11D 3/10; C11D 3/12; C11D 3/37; C11D 3/18; C11D 3/20; B01J 20/04; B01J 20/12; B01J 20/26; B01J 20/24; B01J 20/30; C05G 5/12; A61K 8/02; A61Q 11/00; B01J 2/28; B01J 2/16; A01K 1/015

(52) U.S. Cl. CPC B01J 20/28021; A01K 1/0154; C11D 11/0023; C11D 17/0039; C11D 3/10; C11D 3/126; C11D 3/3707; C11D 3/181; C11D 3/2079; C11D 3/3715; B01J 20/043; B01J 20/12; B01J 20/262; B01J 20/24; B01J 20/3042; B01J 20/3028; C05G 5/12; A61K 8/0279; A61Q 11/00; B01J 2/28; B01J 2/16; C11D 11/0017

(57) ABSTRACT

The present disclosure relates to hollow core granules, products incorporating the hollow core granules, and methods of making the hollow core granules. The hollow core granules can comprise at least one wall surrounding a cavity that is devoid of any solid or liquid so as to define a hollow core, the at least one wall comprising a plurality of individual particles of at least one wall forming material, the plurality of individual particles sufficiently bound together so that the at least one wall is structurally self-sustaining. The hollow core granules further can include a binder material present in the at least one wall. The hollow core granules can be useful as a standalone material and/or may be useful in preparing a variety of products wherein the hollow core granules may be mixed or otherwise combined with further components. The disclosure further provides methods of making such hollow core granules.

### CROSS-REFERENCE TO RELATED APPLICATIONS

The present application claims priority to U.S. Provisional Patent Application No. 62/014,911, filed Apr. 24, 2020, the disclosure of which is incorporated herein by reference in its entirety.

### FIELD OF THE DISCLOSURE

The present disclosure relates to hollow core granules. The hollow core granules can comprise at least one wall surrounding a cavity defining a hollow core. The at least one wall can comprise particles of at least one wall forming material and likewise may include at least one binder.

### BACKGROUND

A variety of chemical compounds are known for a variety of uses in substantially solid forms. Many chemical compounds, when provided in a substantially particulate form, may provide uses that can be limited by the available surface area. For example, while some chemical compounds may be reactive, any reaction may take place substantially only at the surface of the particle, and much of the mass of the particle does not participate in the reaction. Moreover, many materials that are useful in a solid, substantially particulate form may be excessively heavy. For example, clay particles are commonly used in a variety of consumer products, most notably in typical animal litter compositions. Animal litters are often sold in substantially large volumes as may be needed to, for example, fill a litter tray. Because of this typical arrangement, the volume of animal litter that is needed for commercial packaging can be excessively heavy. Still further, there is an ongoing need for new forms of products that provide convenient handling while still exhibiting properties that are equal to or exceed typically achieved ranges. Accordingly, there remains a need in the field for means for providing chemicals, compounds, and compositions in a solid, substantially granular form while also providing improved properties.

### SUMMARY OF THE DISCLOSURE

The present disclosure relates to hollow core granules. The hollow core granules specifically can be an engineered structure wherein a plurality of particles of one or more wall forming materials are aggregated, agglomerated, or otherwise brought together in the form of at least one wall that substantially surrounds a cavity that defines the hollow core. The hollow core granules are differentiated from native forms of the wall forming material in that the combination of individual particles as a wall surrounding a hollow core can cause the granules to exhibit properties that are improved relative to the wall forming material in its native form (i.e., not present as a plurality of particles surrounding a hollow core). This can make the hollow core granules available for a variety of uses in a variety of products including, at least in part, a plurality of the hollow core granules. The disclosure also provides methods of forming such hollow core structures as well as a variety of products or articles of manufacture that can include the hollow core granules.

In one or more embodiments, the present disclosure can relate to a hollow core granule. While the structure is described in relation to a granule in the singular form, it is understood that such terminology is utilized for convenience, and the various properties and uses of the hollow core granule are not limited to a single granule. Rather, a plurality of granules exhibiting substantially the same properties and having substantially the same uses are encompassed by the present disclosure. Moreover, it is understood that, in use, a plurality of the granules will typically be utilized in forming a product or carrying out a specific use. Nevertheless, the present subject matter may be identified in a single granule or a plurality of granules.

In an example embodiment, a hollow core granule according to the present disclosure can comprise at least one wall substantially surrounding a cavity that is substantially devoid of any solid or liquid so as to define a hollow core, the at least one wall comprising a plurality of individual particles of at least one wall forming material, the plurality of individual particles sufficiently bound together so that the at least one wall is structurally self-sustaining. The hollow core granule (or a plurality of the hollow core granules) can be further defined, in one or more embodiments, in relation to any one or more of the following statements, which statements can be combined as desired in any number or order, the ability to make any specific combination of the following statements (or all of the possible combinations of the following statements) being readily evident from the further disclosure herein.

The at least one wall forming material can be selected from the group consisting of clays, glass, ceramics, aluminas, silicates, zeolites, carbon, metals, salts, absorbents, adsorbents, deodorizers, odor control agents, surfactants, enzymes, bleaches, oxidizers, reducers, gellants, flavors, fragrances, abrasives, fertilizers, insecticides, pesticides, bactericides, herbicides, antimicrobials, anti-sticking agents, fillers, binders, preservatives, optical agents, disinfectants, chelators, molecular binding agents, dyes, coloring agents, colored particles, de-dusting agents, and combinations thereof.

The at least one wall forming material includes can include clay.

The clay can comprise bentonite.

The at least one wall forming material can include a salt.

The salt can be selected from the group consisting of calcium carbonate, sodium chloride, sodium carbonate, sodium bicarbonate, sodium percarbonate, sodium sulfate, sodium carbonate peroxide, potassium chloride, magnesium carbonate, magnesium sulfate, and combinations thereof.

The salt can be sodium bicarbonate.

The hollow core granule of claim 6, wherein the salt can be sodium carbonate.

The salt can be sodium chloride.

The at least one wall forming material can be a fabric care composition.

The fabric care composition can be selected from the group consisting of laundry detergents, bleaches, whiteners, brighteners, stain removers, deodorizers, scent boosters, and combinations thereof.

The at least one wall forming material can be an additive for a fabric care composition.

The at least one wall forming material can be a pet litter composition.

The at least one wall forming material can be an additive for a pet litter composition.

The additive for the pet litter composition can be selected from the group consisting of fillers, clumping agents, binders, preservatives, de-dusting agents, fragrances, and mixture thereof.

The at least one wall forming material can be configured for absorption, adsorption, or other binding of one or more odor causing chemicals in which the hollow core granule comes in contact.

The at least one wall forming material can be configured for absorption, adsorption, or other binding of an aqueous liquid in which the hollow core granule comes in contact.

The at least one wall forming material can be configured for absorption, adsorption, or other binding of a non-aqueous liquid in which the hollow core granule comes in contact.

The at least one wall forming material can be a pH adjusting agent.

The at least one wall forming material can include a fertilizer.

The fertilizer can be selected from the group consisting of a nitrogen source, a phosphorus source, a potassium source, a micronutrient source, and combinations thereof.

The hollow core granule as a fertilizer can be characterized by one or more of the following conditions can be met: the at least one wall forming material further can include a clay, and at least a portion of the fertilizer can be absorbed, adsorbed, or otherwise combined with particles of the clay; at least a portion of the fertilizer can be in a microencapsulated form; the fertilizer can include at least two different fertilizers; the fertilizer can be configured for substantially immediate release; the fertilizer can be configured for controlled release.

The at least one wall forming material can include a pesticide.

The pesticide can be an active agent selected from the group consisting of bifenthrin, acephate, carbaryl, cyfluthrin, 2,4-dichlorophenoxyacetic acid, trifluralin, chlorpyrifos, allethrins, cypermethrin, disulfoton, 2,6-dichlorobenzonitrile, metolachlor, cyhalothrin, hydramethylnon, atrazine, chlorothalonil, myclobutanil, dicamba, azadirachtin, captan, diazinon, carbofuran, methomyl, deltamethrin, propiconazole, borate, dinotefuran, dithiopyr, isoxaben, prodiamine, quinclorac, sethoxydim, iron(III) phosphate, mancozeb, thiophanate-methyl, esfenvalerate, tebuconazole, resmethrin, glyphosate, malathion, permethrin, imidacloprid, fipronil, abamectin, spinosad, triclopyr, piperonyl butoxide, pendimethalin, oryzalin, oxadiazon, and combinations thereof.

The at least one wall forming material further can include a clay, and at least a portion of the pesticide is absorbed, adsorbed, or otherwise combined with particles of the clay.

The at least one wall forming material can include an odor masking agent.

The hollow core granule can be hydrophilic.

The hollow core granule can be hydrophobic.

The hollow core granule further can comprise one or more coating layers overlying at least a portion of the at least one wall.

The hollow core granule further can comprise at least one binder material present in at least a portion of interstitial spaces present between the individual particles of the at least one wall forming material.

The at least one binder can be a hydrophilic material.

The at least one binder can include a polyethylene glycol (PEG) material.

The at least one binder can be a hydrophobic material.

The at least one binder can include a material selected from the group consisting of wax, paraffin, polycaprolactone, ethylene-vinyl acetate copolymers, polypropylene carbonate, poly(tetramethylene oxide), poly(ethylene adipate), poly(trans-butadiene), thermoplastic polyurethane, stearic acid, and combinations thereof.

The at least one binder can comprise about 1% to about 45% by weight, based on the total weight of the hollow core granule.

The hollow core granule can have a diameter of about 0.1 mm to about 20 mm.

The diameter of the hollow core granule can be about 0.5 mm to about 6 mm.

The hollow core can have a diameter that is about 10% to about 80% of the diameter of the hollow core granule.

The diameter of the hollow core can be about 25% to about 55% of the diameter of the hollow core granule.

The at least one wall can have an average thickness of about 0.05 mm to about 8 mm. The average thickness can be about 0.1 mm to about 4 mm.

The hollow core granule can be configured so that the cavity that defines the hollow core has a volume that is about 0.1% to about 50% of a volume of the hollow core granule.

The volume of the cavity can be about 0.5% to about 10% of the volume of the hollow core granule.

The hollow core granule can have a density that is at least 20% lower than a density of the wall forming material.

The density of the hollow core granule can be about 15% to about 50% lower than the density of the wall forming material.

The hollow core granule can be buoyant in water.

The at least one wall can be an agglomeration of the individual particles of the wall forming material.

The individual particles of the wall forming material can have an average particle size of about 0.01 mm to about 2 mm.

The individual particles of the wall forming material can have an average particle size of about 0.05 mm to about 1.0 mm. The hollow core granule can exhibit a time to substantially complete solubilization that is at least 10% faster than a time to substantially complete solubilization of a same weight of the at least one wall forming material alone.

The hollow core granule of claim 1, wherein, upon application of an external force, the hollow core granule can be configured to break into a plurality of parts comprising individual groups of the particles of the wall forming material.

In example embodiments, the present disclosure can relate to a product that comprises a plurality of the hollow core granules. The plurality of the hollow core granules can be defined in relation to any one or more of the foregoing statements, as well as any further description of the hollow core granules as described herein. Moreover, the product comprising a plurality of the hollow core granules may be further defined in relation to any one or more of the following statements, which statements can be combined as desired in any number or order, the ability to make any specific combination of the following statements (or all of the possible combinations of the following statements) being readily evident from the further disclosure herein.

The product can be configured as a cleaning product.

The cleaning product can be a fabric care product.

The fabric care product can be selected from the group consisting of laundry detergents, upholstery cleaners, brighteners, whiteners, stain removers, scent boosters, and combinations thereof.

The cleaning product can be a dishwashing detergent.

The cleaning product can be an abrasive cleaner.

The cleaning product can be a teeth cleaning product.

The cleaning product can be a formulation of a plurality of ingredients, and wherein the plurality of the hollow core granule can be configured as a single ingredient of the plurality of ingredients.

The cleaning product can be a formulation of a plurality of ingredients, and wherein two or more of the plurality of ingredients are included as the wall forming material of the plurality of the hollow core granule.

All of the plurality of ingredients can be included as the wall forming material of the plurality of the hollow core granule.

The product can be configured as a nutritional supplement.

The product can be configured as a laxative.

The product can be configured as a deodorizer.

The plurality of the hollow core granule can be configured so as to include a material selected from the group consisting of sodium bicarbonate, zeolites, activated carbon, bentonite, and combinations thereof as the at least one wall forming material.

The plurality of the hollow core granule can be configured so as to include one or both of an odor neutralizing agent and an odor masking agent.

The product can be configured as an animal litter.

The plurality of the hollow core granule can be configured so as to include sodium bicarbonate as the at least one wall forming material.

The plurality of the hollow core granule can be configured so as to include a clay as the at least one wall forming material.

The clay can comprise bentonite.

The plurality of the hollow core granule can comprise at least 5% by weight of the animal litter.

The product can be configured as a pet litter additive.

The pet litter additive can be selected from the group consisting of fillers, clumping agents, binders, preservatives, de-dusting agents, fragrances, and mixture thereof.

The product can be a fertilizer.

The plurality of the hollow core granule can be configured so as to include one or more of a nitrogen source, a phosphorus source, a potassium source, and a micronutrient source as the at least one wall forming material.

The plurality of the hollow core granule can be configured so as to include individual particles of a clay as the at least one wall forming material.

At least one fertilizer material can be absorbed, adsorbed, or otherwise combined with the individual particles of the clay.

The plurality of the hollow core granule can be configured so as to include one or more fertilizer materials in an encapsulated form as the at least one wall forming material.

The product can be a pesticide.

The plurality of the hollow core granule can be configured so as to include an active agent selected from the group consisting of bifenthrin, acephate, carbaryl, cyfluthrin, 2,4-dichlorophenoxyacetic acid, trifluralin, chlorpyrifos, allethrins, cypermethrin, disulfoton, 2,6-dichlorobenzonitrile, metolachlor, cyhalothrin, hydramethylnon, atrazine, chlorothalonil, myclobutanil, dicamba, azadirachtin, captan, diazinon, carbofuran, methomyl, deltamethrin, propiconazole, borate, dinotefuran, dithiopyr, isoxaben, prodiamine, quinclorac, sethoxydim, iron(III) phosphate, mancozeb, thiophanate-methyl, esfenvalerate, tebuconazole, resmethrin, glyphosate, malathion, permethrin, imidacloprid, fipronil, abamectin, spinosad, triclopyr, piperonyl butoxide, pendimethalin, oryzalin, oxadiazon, and combinations thereof.

The plurality of the hollow core granule can be configured so as to include individual particles of a clay as the at least one wall forming material.

At least one pesticide material can be absorbed, adsorbed, or otherwise combined with the individual particles of the clay.

In example embodiments, the present disclosure further can provide methods for preparing hollow core granules. In particular, such methods can comprise: combining a binder having a melting point of about 40° C. to about 95° C. with a plurality of individual particles of at least one wall forming material that is substantially insoluble in the binder and has a melting point that is greater than the melting point of the binder so as to form a mixture; heating the mixture to a maximum temperature that is at or above the melting point of the binder and below the melting point of the plurality of the individual particles of at least one wall forming material to form agglomerations of the plurality of the individual particles of at least one wall forming material; and cooling the agglomerations of the plurality of the individual particles of at least one wall forming material to form the hollow core granules. The methods of manufacture may be further defined in relation to any one or more of the following statements, which statements can be combined as desired in any number or order, the ability to make any specific combination of the following statements (or all of the possible combinations of the following statements) being readily evident from the further disclosure herein.

The hollow core granules that are formed can comprise at least one wall substantially surrounding a cavity that is substantially devoid of any solid or liquid so as to define a hollow core, the at least one wall comprising the plurality of the individual particles of at least one wall forming material, the plurality of the individual particles sufficiently bound together so that the at least one wall is structurally self-sustaining.

The binder and the plurality of the individual particles of at least one wall forming material can be combined such that an amount of the binder present in the at least one wall of the hollow core granules is about 0.1% to about 50% by weight, based on the total weight of the hollow core granules.

The amount of the binder present in the at least one wall of the hollow core granules can be about 5% to about 30% by weight based on the total weight of the hollow core granules.

The process can be carried out in a fluidized bed.

The cooling can comprise cooling to a temperature that is below the melting point of the binder.

The present disclosure, in one or more embodiments, further can relate to products that comprise one or more hollow core granules prepared according to the methods specifically provided above and/or as otherwise described herein. In certain, non-limiting example embodiments, the product can be selected from the group consisting of laundry detergents, dish detergents, fabric cleaners, fabric deodorizers, abrasive cleaners, teeth cleaning compositions, disinfectants, stain removers, whiteners, brighteners, bleaches, scent boosters, absorbents, adsorbents, deodorizers, odor control products, odor masking products, fertilizers, pesticides, animal litters, and animal litter additives.

The present disclosure further comprises methods of delivering one or more materials to a desired site of use, wherein the one or more materials are provided for delivery as a plurality of individual particles of the material included in at least one wall of a hollow core granule as described herein.

### BRIEF SUMMARY OF THE DRAWINGS

FIG. 1 is a partially cut away, perspective view of a hollow core granule according to example embodiments of the present disclosure.

FIG. 2 is a partial cross-sectional of an enlarged portion of a wall of a hollow core granule according to example embodiments of the present disclosure.

FIG. 3 is a partial cross-sectional of an enlarged portion of a wall of a hollow core granule according to further example embodiments of the present disclosure.

FIG. 4 is a cross-sectional view of a hollow core granule incorporating a plurality of walls/layers according to example embodiments of the present disclosure.

FIG. 5 is a graph showing bulk density versus processing time of hollow core granules prepared according to example embodiments of the present disclosure.

FIG. 6 is a graph showing content of the wall forming material versus processing time of hollow core granules prepared according to example embodiments of the present disclosure.

FIG. 7 is a graph showing crush strength versus process time for hollow core granules prepared according to example embodiments of the present disclosure.

FIG. 8A through FIG. 8E are graphs showing attrition for hollow core granules prepared according to example embodiments of the present disclosure at different residence times in a fluidized bed apparatus.

FIG. 9 is a graph showing granule sizes and associated cavity sizes for hollow core granules prepared according to example embodiments of the present disclosure.

FIG. 10 is a graph showing fractional weights of hollow core granules prepared according to example embodiments of the present disclosure with different residence times in a fluidized bed apparatus.

FIG. 11 is a graph showing granule bulk density for hollow core granules with PEG binder and bentonite wall forming material prepared according to example embodiments of the present disclosure with different residence times in a fluidized bed apparatus.

FIG. 12 is a graph showing dimensions and associated cavity dimensions of hollow core granules prepared according to example embodiments of the present disclosure as a function of processing time in a fluidized bed apparatus.

FIG. 13 is a graph showing percentage of cavity volume to total granule volume for hollow core granules prepared according to example embodiments of the present disclosure.

FIG. 14 is a graph showing granule sizes and associated cavity sizes for hollow core granules prepared according to example embodiments of the present disclosure as a function of residence time is a fluidized bed apparatus.

FIG. 15 is a graph showing percentage of cavity volume to total granule volume for hollow core granules prepared according to example embodiments of the present disclosure.

FIG. 16 is a graph showing attrition for hollow core granules prepared according to example embodiments of the present disclosure as a function of time spent in a sieve.

FIG. 17 is a table showing data related to a variety of hollow core granules prepared according to example embodiments of the present disclosure.

FIG. 18 is a table showing additional data related to a variety of hollow core granules prepared according to example embodiments of the present disclosure.

FIG. 19A and FIG. 19B are scanning electron microscope (SEM) images at different magnifications of hollow core particles having a zeolite as the wall forming material according to example embodiments of the present disclosure.

FIG. 20A and FIG. 20B are SEM images at different magnifications of hollow core particles having activated charcoal as the wall forming material according to example embodiments of the present disclosure.

FIGS. 21A, 21B, and 21C are SEM images at different magnifications of hollow core particles having sodium bicarbonate as the wall forming material according to example embodiments of the present disclosure.

FIG. 22 is a graph showing performance of hollow core particles according to example embodiments of the present disclosure versus sodium bicarbonate alone and bentonite alone for reducing malodor caused by release of ammonia from a quantity of the cat litter mimicking composition, Felinine, added to a mass of the tested materials.

FIG. 23 is a graph showing performance of hollow core particles according to example embodiments of the present disclosure versus sodium bicarbonate alone and bentonite alone for reducing malodor caused by release of sulfurous compounds from a quantity of the cat litter mimicking composition, Felinine, added to a mass of the tested materials.

FIG. 24 is an image of a hollow core granule formed of sodium bicarbonate as the wall forming material and PEG as the binder according to example embodiments of the present disclosure, the granule having been cut in half.

FIG. 25 is an image of a hollow core granule formed of bentonite as the wall forming material and PEG as the binder according to example embodiments of the present disclosure, the granule having been cut in half.

FIG. 26 is an image of a bass of hollow core granules formed of sodium bicarbonate and bentonite as the wall forming material and PEG as the binder according to example embodiments of the present disclosure.

FIG. 27 is an image of a hollow core granule formed of sodium bicarbonate as the wall forming material and polyoxyethylene stearyl ether as the binder according to example embodiments of the present disclosure, the granule having been cut in half.

FIG. 28 is an image of a hollow core granule formed of bentonite as the wall forming material and polyoxyethylene stearyl ether as the binder according to example embodiments of the present disclosure, the granule having been cut in half.

### DETAILED DESCRIPTION OF THE DISCLOSURE

The invention now will be described more fully hereinafter through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The present disclosure relates to structures having substantially hollow cores, compositions incorporating such structures, methods of making such structures, and uses/applications of such structures and compositions. The structures provided herein may particularly be hollow core structures comprising at least one shell/wall surrounding a cavity, which is the hollow core. The shell/wall specifically can comprise at least one solid wall forming material and a binder material. A plurality of solid wall forming materials may be utilized. Likewise, a plurality of binders may be utilized. The at least one solid wall forming material can be configured as a plurality of individual particles that are bound together with the binder to define the shell/wall surrounding the cavity defining the hollow core. The shell/wall may be characterized as a substantially continuous wall that surrounds and encloses a hollow core or cavity. An individual hollow core structure, the wall being formed of a plurality of individual particles, may thus be referenced as being a granule. Thus, the term “granule” as used herein can reference a hollow core structure, and the term “particle” can reference individual pieces of the solid material(s) used as the wall forming material to form the shell/wall of the granule or hollow core structure. In some embodiments, a plurality of shells/walls can be present, and each shell/wall can independently have a different composition and/or thicknesses. Further, the hollow core may be configured such that one or more components are included therein in an amount such that the hollow core is not completely filled and thus can still be referenced as being a hollow core. Such hollow core structures may be useful as a standalone material and/or may be useful in preparing a variety of products wherein the hollow core structures may be mixed or otherwise combined with further components.

Hollow core granules according to the present disclosure can be configured with specific properties and specific uses. The exact nature of the properties and/or uses can vary based upon, among other factors, the nature of the material(s) forming the shell(s)/wall(s), the size of the hollow core structures, the nature of any material forming a component that is included within the hollow core, and the like. In some embodiments, the present hollow core structures can be particularly configured to provide one or more deodorization functions. This can include exhibiting an ability to absorb and/or entrain odor-causing compounds and can alternatively or additionally include exhibiting an odor neutralizing ability, such as by including and/or delivering an odor neutralizing agent. In some embodiments, the present hollow core structures can be particularly configured to provide one or more absorbent and/or adsorbent functions. This can include exhibiting an ability to absorb liquids, which can include polar and/or non-polar liquids. Further, the hollow core structures can be configured to selectively absorb and/or adsorb in terrestrial and/or aquatic settings.

In some embodiments, the present hollow core structures can be provided as an engineered form of one or more chemicals, compounds, compositions, or the like that has a desired use, and the provision of the one or more chemicals, compounds, compositions, or the like in the hollow core format can achieved improved properties (e.g., hollow core sodium carbonate exhibiting improved odor absorption and/or cleaning properties relative to “regular” sodium bicarbonate that is not in the re-engineered, hollow core format or hollow core clay exhibiting improved liquid absorption relative to “regular” clay that is not in the re-engineered, hollow core format). The improved properties particularly may be as related to a native form of the chemical, compound, composition, or the like, the native form being a form in which the chemical, compound, or composition naturally exists, or a form in which the chemical, compound, or composition is typically made and/or sold. The native form in particular can be a form that is not a hollow core format.

The present hollow core structures can be useful as a stand-alone chemical or compound that can be made available for a variety of purposes. Likewise, such stand-alone chemicals or compounds may be used as one or more components of a more complex composition (e.g., the complex composition being a material this is formed of at least two different chemicals, compounds, or the like). Further, two or more chemicals, compounds, or the like may be combined to form hollow core granules, which granules may form part or all of a composition. In example embodiments, stand-alone chemicals, compounds, or the like can include materials, such as sodium bicarbonate, clays, surfactants, etc., and further examples of such materials are further discussed herein. Such materials can thus be provided as a product that is formed entirely or in part of granules prepared such material. For example, cleaning products, abrasives, personal care products, deodorizers, animal litters, and the like may be prepared in total from hollow core granules as described herein or, alternatively, such hollow core granules may form one or more components of such products. In some embodiments, the present hollow core structures can be particularly configured for use in delivery of a desired product to a desired setting. For example, fertilizers, pesticides, and the like can be provided as hollow core structure to enable delivery of the fertilizers, pesticides, and the like with improved properties. The foregoing uses and products are understood to be example embodiments and are not intended to be limiting of the useful applications of the presently disclosed hollow core structures.

Structures with Hollow Cores

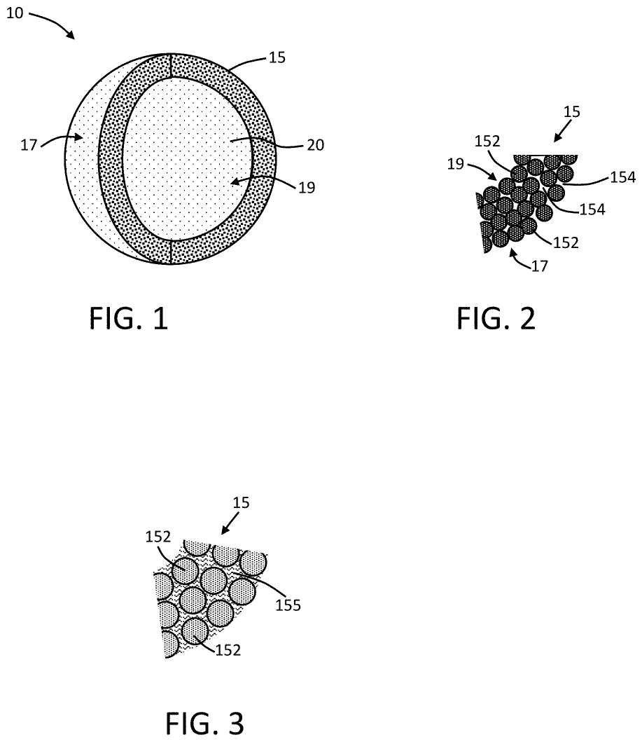

Referring to FIG. 1, a structure/granule 10 according to the present disclosure may comprise an outer wall 15 surrounding and substantially enclosing an inner core 20, which inner core may be substantially hollow and thus define a cavity. It is understood that the term “wall” should not be construed as limiting, and such term may be synonymous with similar terms, such as “shell.” Thus, while the term “wall” may be used throughout this disclosure, it is understood that the wall surrounds the cavity defining the hollow core. A substantially hollow core may include a relatively small content of material (e.g., solid or liquid) but is otherwise essentially an open void within the outer wall. Specifically, the phrase “substantially hollow” may indicate that at least 90%, at least 95%, or at least 99% by volume of the core is void of any solid and/or liquid material. The structure 10 may be further defined in relation to having an outer wall surface 17 and an inner wall surface 19. The core of the hollow core structure thus may be defined as the interior volume of the hollow core structure that is bounded by the inner wall surface 19.

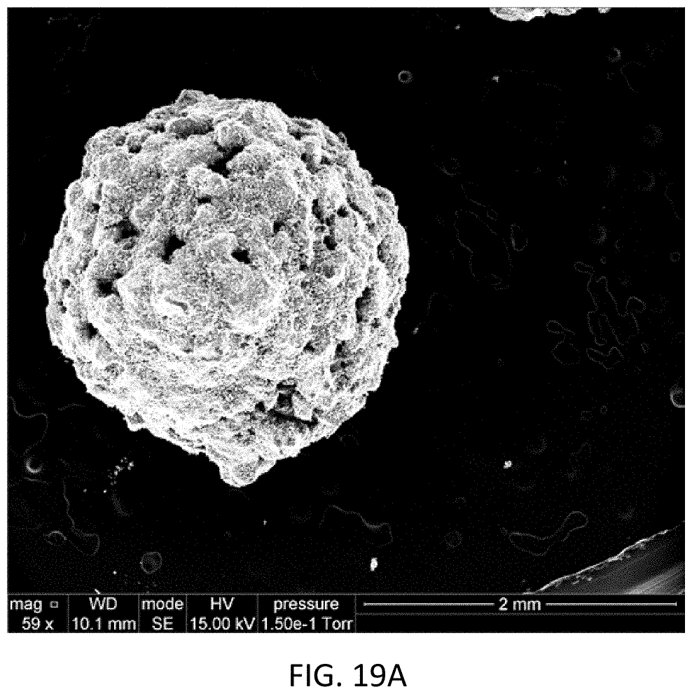

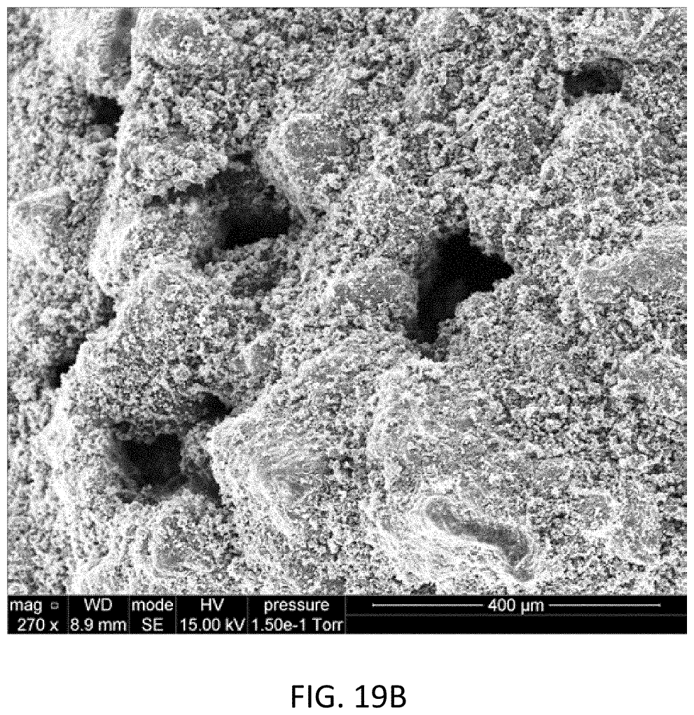

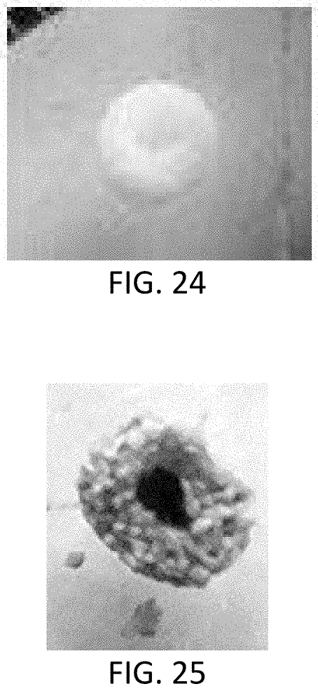

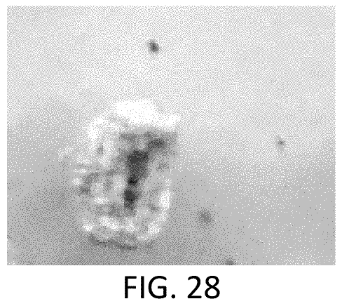

A hollow core granule according to the present disclosure particularly may be described as comprising at least one wall substantially surrounding a cavity that is substantially devoid of any solid or liquid so as to define a hollow core, the at least one wall comprising a plurality of individual particles of at least one wall forming material, the plurality of individual particles sufficiently bound together so that the at least one wall is structurally self-sustaining. The at least one wall that is substantially surrounding a cavity can indicate that the wall completely surrounds the cavity or may indicate the open porosity of the wall in that one more open pores may define one or more pathways between the inner cavity and the outer environment. In addition to further discussion provided herein, the nature of the at least on wall “substantially surrounding” the cavity particularly can mean that the wall completely surrounds the cavity (i.e., 100% enclosure) or surrounds the cavity with a minor portion of the wall being discontinuous, such as through the presence of open pores or other discontinuities in the wall providing an opening between the cavity and the outer environment (i.e., at least 90%, at least 95%, at least 98%, or at least 99% enclosure based on the area of the wall) may be present. Identifying the amount of enclosure can be calculated based on measurements of microscopic images. For example, in the SEM images provided in FIG. 19A to FIG. 21C, it is evident that open pores in the wall can be visually identified and measured. Other analytic methods may similarly be used. In some embodiments, it can be desirable to have somewhat less than 100% enclosure of the cavity in order to achieve improved properties as described herein. The cavity being “substantially devoid” of any solid or liquid can indicate that the core of the granule is not intentionally filled with solid or liquid material and there is an open space spanning across the hollow core granule when measured from an interior surface of the wall. This is particularly evident in the images shown in FIG. 24, FIG. 25, FIG. 27, and FIG. 28 of hollow core granules that have been cut in half to show the interior cavity. Substantially devoid thus can mean that internal volume of the core as defined by the inner surface of the wall is at least 90%, at least 95%, at least 97%, or at least 99% open and absent of any solid or liquid. The plurality of individual particles being “sufficiently bound” can mean that particles retain their positioning relative to one another and do not exhibit any significant degree of rearrangement during normal handling of the hollow core granules.

The hollow core structure 10 may be provided in a variety of sizes, and an average size may be defined in relation to a diameter (e.g., for substantially spherical structure) or in relation to the largest dimension (e.g., a cross-wise or length-wise measurement for a substantially elongated or non-uniform structures) of the hollow core structure. The hollow core granules can have an average size of about 0.1 mm to about 20 mm, about 1 mm to about 10 mm, or about 2 mm to about 5 mm. In some embodiments, the hollow core structures may be substantially small in size, such as having an average size of about 0.1 mm to about 7 mm, about 0.5 mm to about 6 mm, about 1 mm to about 5 mm, about 1.5 mm to about 4.5 mm, or about 2 mm to about 4 mm. In other embodiments, the hollow core structures may be substantially larger in size, such as having an average size of about 2 mm to about 20 mm, about 3 mm to about 15 mm, or about 4 mm to about 12 mm. In still further embodiments, even larger sizes can be achieved, such as about 5 mm to about 50 mm, about 10 mm to about 45 mm, or about 15 mm to about 40 mm. The foregoing sizes may thus relate to an individual granule. Further, as is more evident from the methods of manufacture described below, the granule size achieved may be defined at least in part by the particle size of the binder material that is used. Thus, binder material may be provided in larger particle sizes to achieve larger hollow core granules, and binder material may be provided in smaller particle sizes to achieve smaller hollow core granules.

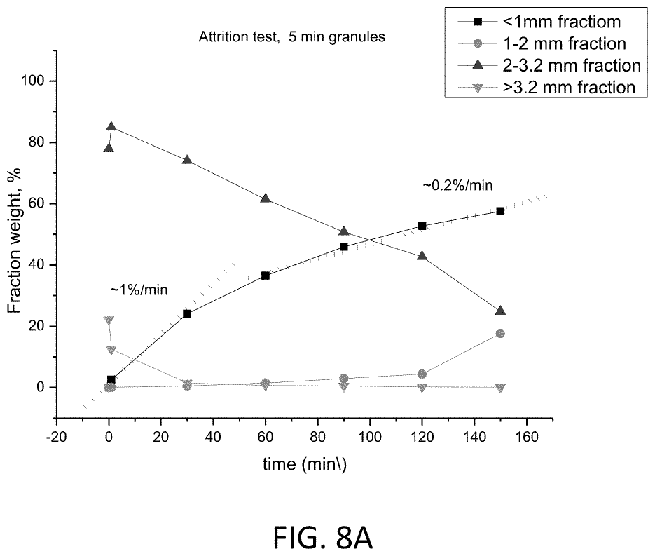

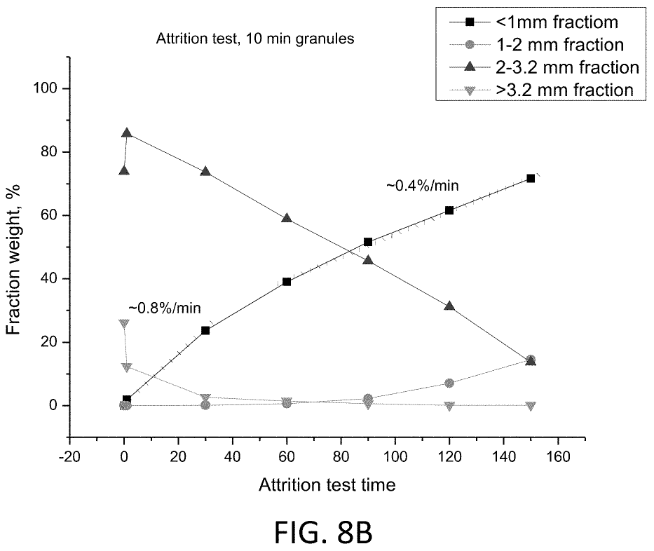

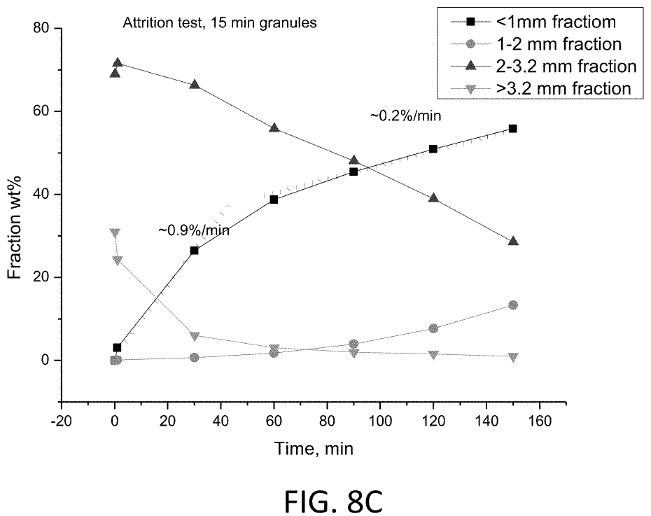

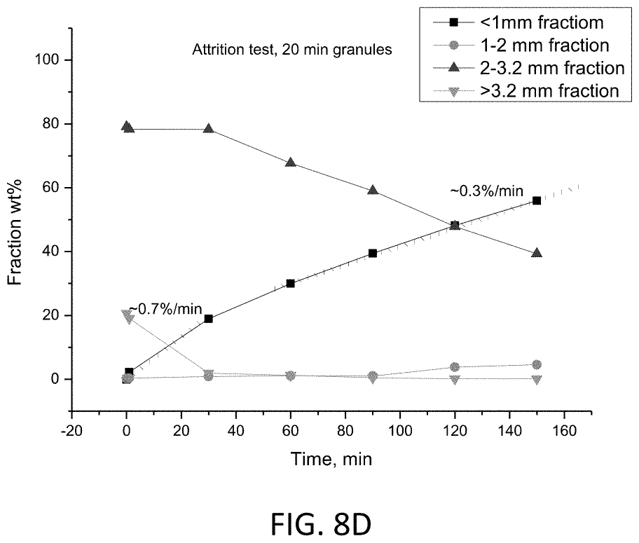

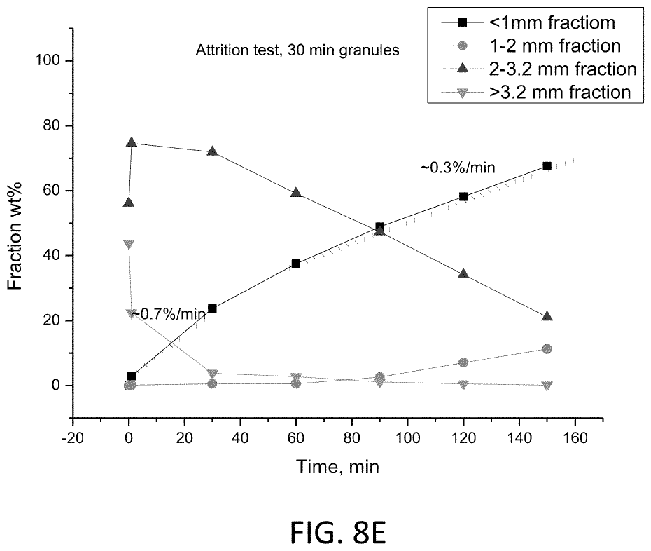

In some embodiments, individual granules of the hollow core structures may be substantially spherical, substantially elliptical, or may otherwise have a substantially rounded form. In such embodiments, the wall may completely surround the cavity defining the hollow core (i.e., the hollow core is completely separated from the surrounding environment). Other shapes, however, are not excluded. For example, in certain embodiments, a hollow core structure 10 as provided herein may be in an elongated form, such as a substantially fibrous form or a tubular form, which may have a closed end, an open end, or a partially closed end. Further, the structures 10 may be substantially irregular in form. For example, hollow core granules may have a substantially ellipsoid shape. Moreover, at least a portion of the wall of the hollow core granule may be concave. In some embodiments, a plurality of structures 10 may adhere one to another to form agglomerations of two, three, four, or more structures. Such agglomerates may have a substantially “pear” shape (e.g., where two adhered particles are of differing sizes) or may have a substantially “FIG. 8” shape (e.g., where two adhered particles are of substantially the same size).

As illustrated in FIG. 1, the wall 15 of the structure 10 is substantially uniform in thickness. In some embodiments, however, the thickness of the wall 15 may vary. An average wall thickness (e.g., measured from the outer wall surface 17 to the inner wall surface 19) may be in the range of about 0.05 mm to about 8 mm, about 0.1 mm to about 7 mm, about 0.5 mm to about 6 mm, about 1.0 mm to about 5 mm, or about 1.5 mm to about 2.5 mm. When small size granules are prepared, the average wall thickness thereof may be proportionally smaller, such as about 0.1 mm to about 4 mm, about 0.25 mm to about 3.5 mm, about 1 mm to about 3 mm, or about 1.5 mm to about 2.5 mm. The wall thickness and the overall size of the hollow core structures 10 can vary based upon the types of materials used in forming the hollow core structures. In particular, the nature of the binder that is used can strongly influence the size of the cavity that defines the core of the hollow core structure. Likewise, the wall thickness can depend at least in part on the size of individual particles of wall forming material that are used. In some embodiments, processing conditions, such as the length of time spent in the fluidized bed, can also factor into the dimensions of the hollow core structures. Thus, one may customize the relative dimensions of the overall size of the hollow core structure, the thickness of the wall of the hollow core structure, and the size of the cavity defining the hollow core of the structure through choice of binder material, choice of the type of wall forming material, and size of the individual particles of the wall forming material. In some embodiments, such dimensions can be summarized in relation a diameter of the cavity defining the hollow core (i.e., the diameter across the hollow core at the largest dimension as measured at inner wall surface) of an individual granule relative to an overall diameter of the individual granule (i.e., the diameter across the granule at the largest dimension as measured at the outer wall surface). In particular, the cavity diameter can be about 10% to about 80%, about 15% to about 65%, about 20% to about 60%, about 25% to about 55%, or about 30% to about 50% of the diameter of the granule. In some embodiments, the relative dimensions can be summarized in relation to a volume of the cavity defining the hollow core to a volume of the overall granule. In particular, the cavity volume can be about 0.1% to about 50%, about 0.25% to about 25%, about 0.5% to about 10%, about 0.7% to about 7%, or about 1% to about 4% of the volume of the overall granule. The relative dimensions noted above can also affect the bulk density of the hollow core structures. In various embodiments, hollow core structures as described herein can have a bulk density in the range of about 200 grams per liter (g/L) to about 2000 g/L, about 250 g/L to about 1200 g/L, about 200 g/L to about 900 g/L, about 400 g/L to about 850 g/L, about 450 g/L to about 800 g/L, or about 500 g/L to about 750 g/L. As such, hollow core granules as described herein can have a bulk density that is significantly different from the bulk density of the wall forming material itself. For example, where sodium bicarbonate has a bulk density of about 1100 g/L, hollow core granules as described herein using sodium bicarbonate as the wall forming material can have a bulk density of about 700 g/L. Similarly, where bentonite has a bulk density of about 1000 g/L, hollow core granules as described herein using bentonite as the wall forming material can have a bulk density of about 600 g/L. Accordingly, in some embodiments, hollow core granules of the present disclosure can have a bulk density that is lower than the bulk density of the wall forming material in its native form (i.e., as found in nature or as sold as a commodity product) by at least 20%, at least 30%, or at least 40%. Specifically, the hollow core granules can have a bulk density that is about 10% to about 75%, about 15% to about 50%, or about 20% to about 45% lower than the bulk density of the wall forming material in its native form. The comparison may be characterized as being the density of the formed, hollow core granule versus the density of the wall forming material prior to being incorporated into the hollow core granule.

The present hollow core structures, despite having an open or substantially open cavity bounded by a wall, can still retain a substantially consistent shape. This is a surprising effect since the at least one is formed of a plurality of individual particles of the wall forming material without an interior mass supporting the wall. Accordingly, the at least one wall can be characterized as being substantially self-sustaining in that the wall does not substantially cave-in on itself but rather maintains a granule shape as described above while having a central cavity that, in some embodiments, is substantially devoid of any solid or liquid material therein.

The hollow core granules, despite being hollow rather than being solid throughout the granule, can still exhibit significantly high strength. The strength in particular may be a crush strength, such as discussed in the appended examples. The strength can vary based upon the choice of wall forming material and the choice of binder. In some embodiments, granule strength can be at least 0.5 Newtons (N), at least 2 N, at least 3 N, at least 5 N, at least 10 N, or at least 15 N. The maximum granule strength, in some embodiments, may have a maximum value of about 50 N. In certain embodiments, granule strength can be about 0.5 N to about 50 N, about 1 N to about 30 N, about 2 N to about 25 N, or about 3 N to about 20 N.

The wall 15 of the hollow core structures 10 being configured as an agglomeration of individual particles 152 of one or more solid, wall-forming materials causes the wall 15 to have interstitial spaces 154 between the particles 152. This is seen in the partial cross-section illustrated in FIG. 2. As such, the wall 15 is a substantially continuous structure in that it is formed of individual particles that are sufficiently associated together to form a stable, self-sustaining structure, the interstitial spaces can provide certain properties to the hollow core structures 10. As seen in FIG. 2, the outer surface 17 and/or the inner surface 19 of the wall 15 is not necessarily uniform and may exhibit a level of roughness or unevenness that may be differentiated from a substantially smooth wall surface. In some embodiments, the interstitial space 154 may be at least partially filled with a binder material. This is shown in FIG. 3, wherein the particles 152 are substantially surrounded by a binder 155. It is understood, however, that the binder 155 may not necessarily completely surround each and every particle 152. Likewise, the binder 155 may be present in a discontinuous form, such as a particulate form such that individual binder particles may bind together two or more particles 152 of the wall-forming material.

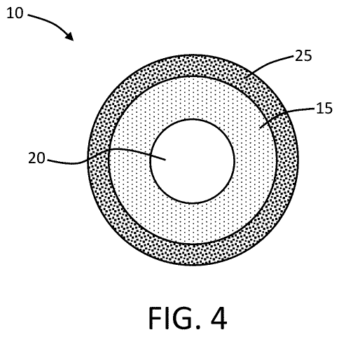

A hollow core structure 10 according to the present disclosure may comprise a single wall 15. In some embodiments, however, the structure 10 may be provided with a plurality of walls, which may be characterized as a wall with a multi-layer construction in some embodiments. As seen in the cross-section of FIG. 4, the structure 10 may comprise an inner core or cavity 20 that can be substantially empty or devoid of solid or liquid material and a surrounding wall 15. The wall 15 (which can be referenced as a first wall, first layer, inner wall, or inner layer) may then be substantially surrounded by another wall 25 (which may be referenced as a second wall, second layer, further wall, further layer, outer wall, or outer layer). As such, the hollow core structure 10 may comprise a single wall or layer surrounding the substantially hollow inner core 20 or may comprise a plurality of walls or layers. When a plurality of walls or layers is present, each individual wall or layer may have a different average thickness, or the relative average thicknesses of the walls or layers may vary. In some embodiments, an outer wall or layer may have a lesser average thickness than an inner wall or layer. At least one of the plurality of walls or layers will be an agglomeration of individual particles of the wall-forming material. One or more walls or layers, however, and particularly an outer wall or outer layer may be configured as a coating that is applied to an inner wall or inner layer. Agglomeration may be referenced more particularly in relation to substantial adhesion of the individual particles of the wall forming material to adjacent particles. The adhesion may occur due to a variety of interactive forces and may be achieved at least in part due to the presence of the binder material that is one or more of at least partially coating individual particles of the wall forming material and/or that is at least partially filling the interstitial spaces between the individual particles of the wall forming material.

In some embodiments, a hollow core structure as described herein may be defined in relation to a porosity of a wall of the structure. Porosity may be at least partially defined in relation to the presence of the interstitial spaces 154 between particles 152 forming the wall 15 of an individual granule of the hollow core structures 10. Porosity may be controlled in a variety of manners, such as by altering the average size of individual particles 152 forming the wall 10, by combining particles of two or more different average particle sizes, by controlling the amount of any binder that may be present, and the like. For example, particles used as wall forming materials may have an average size in the range of about 0.01 mm to about 2 mm, about 0.02 mm to about 1.5 mm, about 0.05 mm to about 1.0 mm, or about 0.1 mm to about 0.8 mm. In some embodiments, a range of particles sizes may be used to achieve a greater packing density in the wall with smaller particles filling in spaces between larger particles. Thus, particles of the wall forming material may have an average size spanning a range so that the smallest particle size differs from the largest particle size by about 1 mm, about 0.8 mm, about 0.5 mm, or about 0.2 mm.

In some embodiments, porosity further may be at least partially controlled through choice of material used in forming the wall, such as utilizing a material with a high or low porosity or utilizing combinations of materials with different porosities. Example materials useful in forming the walls of the present, hollow core structures are discussed in detail below. In some embodiments, porosity may be defined in relation to any one or more of average pore size, pore distribution, and the like. For example, an average pore size of pores in the wall of the structure may be in the range of about 100 nm to about 200 μm, about 250 nm to about 100 μm, or about 500 nm to about 50 μm.

In addition to the nature of the walls, hollow core structures according to the present disclosure likewise can be defined in relation to the nature of the hollow cores. As noted above, the cavity (i.e., an open volume) defining the hollow core can vary, and the cavity may be substantially completely devoid of any solid or liquid materials (e.g., less than 10%, less than 5%, less than 2%, or less than 1% of the cavity volume including any solids or liquids therein at the time of manufacturing). In some embodiments, a hollow core structure may include a content of a further material present in the volume defined by the inner surface of the innermost wall of the hollow core structure. For example, structural scaffolding may be present in the cavity defining the hollow core. As other examples, liquids may be filled in the cavity defining the hollow core. As such, the hollow core structures may provide delivery articles whereby a material present in the hollow core can be delivered in a controlled manner via dissolution, breakage, or other removal of the outer wall to release the inner material.

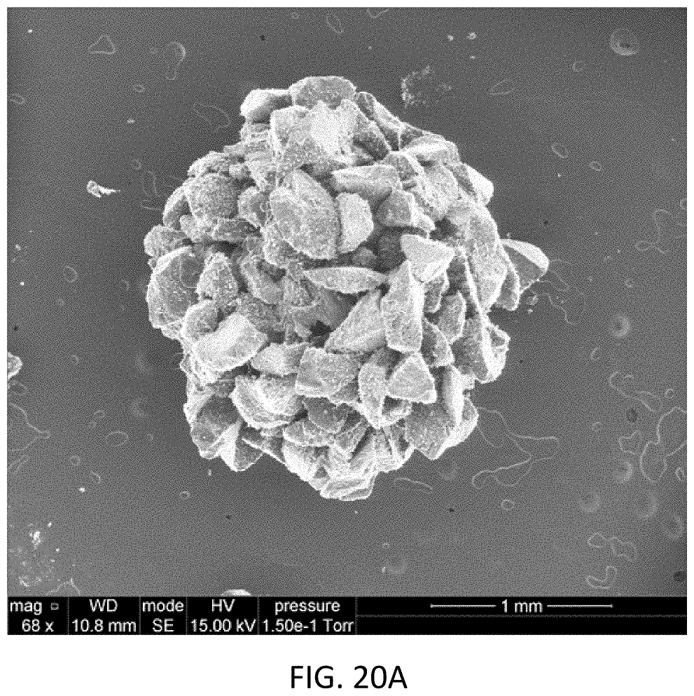

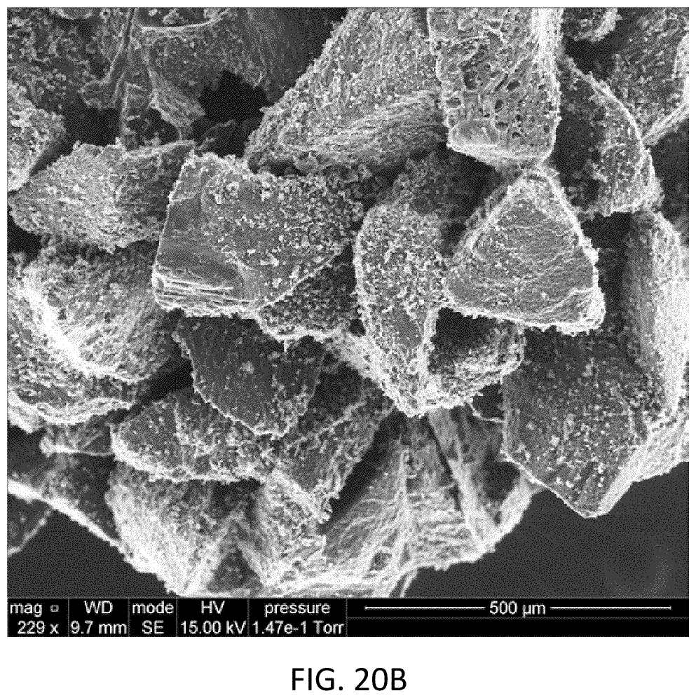

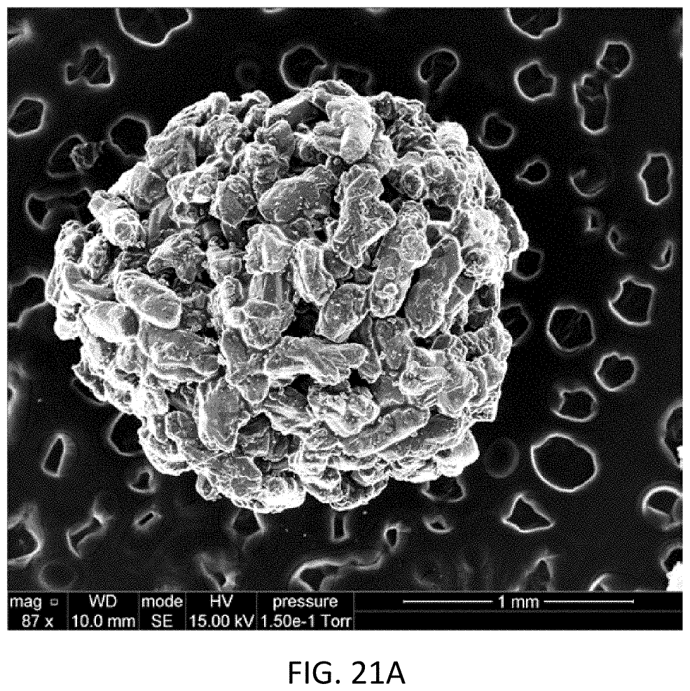

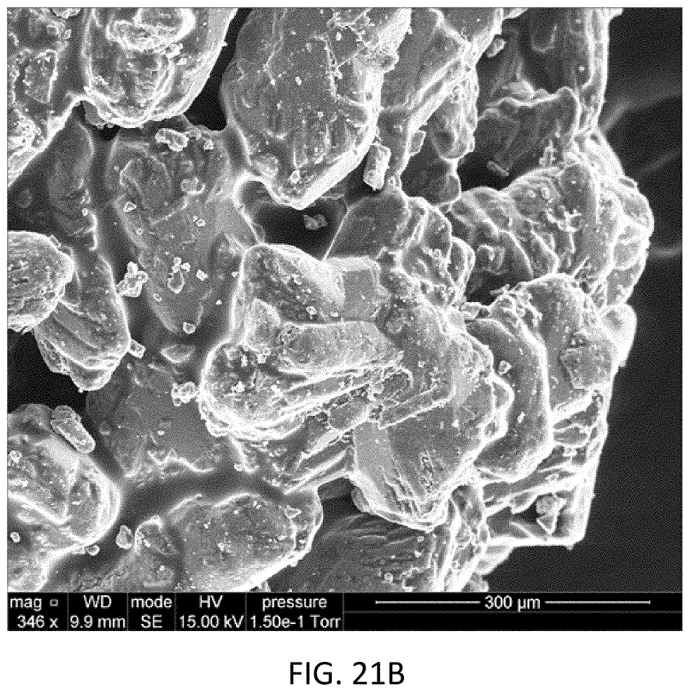

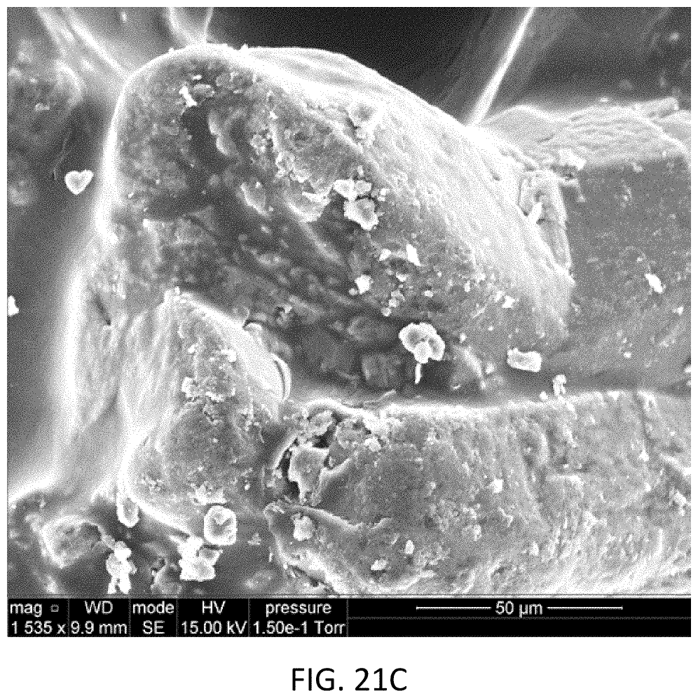

Structures according to the present disclosure may be characterized utilizing a variety of testing techniques. For example, scanning electron microscopy (SEM) testing may be useful in characterizing the particle characteristics, particle morphology, porosity and pore distribution, and the like. Accordingly, the present structures and products incorporating such structures may be further defined in relation to one or more of the foregoing characteristics. Porosity of the hollow core granules can be seen, for example, in the scanning electron microscope (SEM) images shown in FIG. 19A to FIG. 21C. A hollow core granule comprising zeolite particles as the wall forming material is shown in the SEM images of FIG. 19A at 59× magnification and in FIG. 19B at 270× magnification. A hollow core granule comprising activated charcoal particles as the wall forming material is shown in the SEM images of FIG. 20A at 68× magnification and in FIG. 20B at 229× magnification. A hollow core granule comprising sodium bicarbonate particles as the wall forming material is shown in the SEM images of FIG. 21A at 87× magnification, in FIG. 21B at 346× magnification, and in FIG. 21C at 1,535× magnification. As seen in the respective images, hollow core granules were consistently prepared with particles of different wall forming materials. Further, it is apparent from the images that the hollow core granules consistently retain a similar structure with the wall of the hollow core granule having a number of pores between the individual particles of the wall forming material. Open porosity is seen to be variable with more of less of the pores being filled with the binder material. Thus, the hollow core granules can be configured with greater or lesser open porosity by controlling processing so that more or less of the binder is retained in the walls of the hollow core granules. The ability to control open porosity can be valuable in fine tuning the properties achieved, such as improved solubilization, absorption/adsorption properties, and other properties as further discussed herein.

A variety of wall-forming materials may be used to prepare one or more walls of a hollow core granule according to the present disclosure. Wall forming materials may be one or both of functional and structural. A functional, wall forming material can be any material that is included in the hollow core granule to provide a desired function to a product comprising the hollow core granules. Accordingly, such materials may be used individually to form a product exhibiting the function of the functional material and/or may be used in any combination of any number of such materials to form a product exhibiting the combined functions. It is understood that products including hollow core granules having one or more of the functional materials as a wall forming material may also include other, non-functional components, such as fillers, bulking agents, inert components, and the like. Moreover, the hollow core granules themselves may include fillers, bulking agents, inert components, or the like as one or more wall forming materials in combination with one or more functional materials to achieve the proper dosing of the functional material in the overall hollow core granules. The functional material(s) may be available is a solid form (e.g., particles) under conditions necessary for preparation of a hollow core granule as described herein. In such cases, the functional material may be additionally effective as a structural component of the wall(s) of the hollow core granule. In some embodiments, however, one or more functional materials for use in the hollow core granules may be typically available in a liquid form under conditions necessary for preparation of a hollow core granule as described herein. In such embodiments, the liquid material may be combined with a structural material to provide the liquid in a solid form. A structural material with which one or more liquid materials may be combined may also be a functional material. A structural material with which one or more liquid materials may be combined, however, may be non-functional in the hollow core granule to be prepared, and the structural material may thus be referenced as a carrier component or particle, a filler, a bulking agent, an inert component or particle, or the like. Clays, ceramics, silicates, zeolites, carbon, and even other minerals or salts can be useful as carriers into or onto which a desired liquid for inclusion in the hollow core granules can be absorbed, adsorbed, or impregnated. The carrier particles may be considered substantially inert to the delivery site (i.e., providing no desired benefit but still safe for use) and may remain after delivery of the active agent or may further dissolve or disintegrate. In some embodiments, the carrier particles may provide an additive effect so that the efficacy of the liquid, functional material is improved through combination with the carrier particles or so that the carrier particles themselves provide a different, desired effect at the delivery site.

Liquid components may alternatively, or additionally, be provided in a form suitable for use in forming one or more walls of a hollow core granule as described herein through use of encapsulation technologies. Thus, capsules and/or microcapsules may be utilized. Encapsulation technologies likewise can be utilized with otherwise solid materials in order to provide the encapsulated component in a controlled release form whereby an encapsulating shell must be solubilized, degraded, or otherwise removed in order for the encapsulated material to be released at a delivery site.

Encapsulation of any material to be used as a wall forming material for the present hollow core granules can be carried out using any suitable technique. For example, microcapsules can be formed using any of various chemical encapsulation techniques such as solvent evaporation, solvent extraction, organic phase separation, interfacial polymerization, simple and complex coacervation, in-situ polymerization, liposome encapsulation, and nanoencapsulation. Alternatively, physical methods of encapsulation could be used, such as spray coating, pan coating, fluid bed coating, annular jet coating, spinning disk atomization, spray cooling, spray drying, spray chilling, stationary nozzle coextrusion, centrifugal head coextrusion, or submerged nozzle coextrusion. Regardless of the encapsulation methodology employed, materials used to form the capsules can vary. Classes of materials that are typically used as wall or shell materials include proteins, polysaccharides, starches, waxes, fats, natural and synthetic polymers, and resins. Exemplary materials for use in the microencapsulation process used to form the microcapsules include gelatin, acacia (gum arabic), polyvinyl acetate, potassium alginate, carob bean gum, potassium citrate, carrageenan, potassium polymetaphosphate, citric acid, potassium tripolyphosphate, dextrin, polyvinyl alcohol, povidone, dimethylpolysiloxane, dimethyl silicone, refined paraffin wax, ethylcellulose, bleached shellac, modified food starch, sodium alginate, guar gum, sodium, sodium citrate, carboxymethylcellulose, hydroxypropyl cellulose, hydroxypropylmethylcellulose, sodium ferrocyanide, sodium polyphosphates, locust bean gum, methylcellulose, sodium trimetaphosphate, methyl ethyl cellulose, sodium tripolyphosphate, microcrystalline wax, tannic acid, petroleum wax, terpene resin, tragacanth, polyethylene, xanthan gum, and polyethylene glycol. Microcapsules are commercially available, and exemplary types of microcapsule technologies are of the type set forth in Gutcho, Microcapsules and Microencapsulation Techniques (1976); Gutcho, Microcapsules and Other Capsules Advances Since 1975 (1979); Kondo, Microcapsule Processing and Technology (1979); Iwamoto et al., AAPS Pharm. Sci. Tech. 2002 3(3): article 25; U.S. Pat. No. 5,004,595 to Cherukuri et al.; U.S. Pat. No. 5,690,990 to Bonner; U.S. Pat. No. 5,759,599 to Wampler et al.; U.S. Pat. No. 6,039,901 to Soper et al.; U.S. Pat. No. 6,045,835 to Soper et al.; U.S. Pat. No. 6,056,992 to Lew; U.S. Pat. No. 6,106,875 to Soper et al.; U.S. Pat. No. 6,117,455 to Takada et al.; U.S. Pat. No. 6,482,433 to DeRoos et al.; and U.S. Pat. No. 6,929,814 to Bouwmeesters et al.; each of which is incorporated herein by reference.

Non-limiting example embodiments of materials that can be suitable for use in forming a wall of a hollow core structure as described herein can include clays (e.g., bentonite), glass, ceramics, aluminas, silicates, zeolites, carbon (e.g., activated charcoal), metals, salts (e.g., sodium bicarbonate or baking soda, sodium carbonate or soda ash, sodium chloride, etc.), powdered formulations (e.g., solid cleaning compositions, such as laundry detergents, dish detergents, fabric cleaners/deodorizers, abrasive cleaners, etc.), absorbents, adsorbents, deodorizers, odor control agents, health or beauty agents, surfactants, enzymes, bleaches, oxidizers (e.g., peroxides), reducers, gellants (e.g., gelatin, pectin, cellulosics, etc.), flavors, fragrances, abrasives, fertilizers, insecticides, pesticides, bactericides, herbicides, antimicrobials, anti-sticking agents, fillers, binders, preservatives, optical agents (e.g., brighteners), disinfectants, chelators, molecular binding agents, dyes, coloring agents, colored particles, de-dusting agents, and other materials known for use in consumer products and/or industrial settings to provide a specific function to a product. Any of the foregoing may be a functional material as referenced above and may also be referenced as additives in that they can be added to other products to impart the desired function and/or may be provided as a stand-alone product that can be combined as needed with other products to achieve an additive result. Such materials may be used in a solid form as a functional and/or structural wall forming material without modification or with modification to impart controlled release and/or to modify hydrophilicity/hydrophobicity of the material. Such materials may be used in a liquid form as a functional wall forming material when combined with a carrier or other solid material and/or with modification to be in a solid format, such as encapsulation techniques noted above. The foregoing list of wall forming materials is not intended to be all-inclusive, and it is understood that the skilled person, in light of the totality of the present disclosure, will be able to identify other chemicals, compounds, compositions, and the like that are used in or as commercial products that can likewise be utilized in forming hollow core structures as presently disclosed.

In certain embodiments, bentonite or sodium bicarbonate may be particularly used as a wall forming material due to the extensive number of uses for such materials, and either may be utilized as a functional and/or structural component of the present hollow core granules. Non-limiting examples of bentonite clays that can be used include sodium bentonite, potassium bentonite, lithium bentonite, calcium bentonite and magnesium bentonite, or combinations thereof. Clay-based liquid absorbing materials are described, for example, in U.S. Pat. No. 8,720,375 to Miller et al., the disclosure of which is incorporated herein by reference. Further, non-limiting examples of absorbent or adsorbent materials that are suitable for use in hollow core granules in combination with, or as an alternative to bentonite can include clay, quartz, feldspar, calcite, illite, calcium carbonate, carbon, mica, Georgia white clay, hectorite, smectite, opal, kaolinite, pumice, tobermite, slate, gypsum, vermiculite, halloysite, sepiolite, marls, diatomaceous earth, dolomite, attapulgite, montmorillonite, Monterey shale, Fuller's earth, silica, fossilized plant materials, perlites, expanded perlites, mixtures thereof, and like materials.

Preferably, the wall forming materials at the time of preparation of the hollow core granules will be in a solid, substantially particulate form and likewise may be adapted to or configured to be substantially insoluble in a binder that may be used in forming the walled structure. This may reference a naturally occurring state of the material or may arise through combination of the desired material with another structural material as already discussed above. In some embodiments, the wall-forming material, when used in preparation of the hollow core granules, will be configured as solid particles with a melting point of about 100° C. or greater, about 110° C. or greater, about 120° C. or greater, or about 130° C. or greater.

Any functional material in a hollow core granule according to the present disclosure may be provided in a manner so as to provide controlled release of the material. Controlled release can specifically indicate any of the following: delayed release so that, after a defined period, substantially the entire amount of the material (i.e., a “bolus”) is released; delayed release so that, after a defined period, release of the material begins and proceeds over a second defined period (i.e., “prolonged release”); or metered release so that release of the material begins substantially immediately after application, but the release proceeds over a defined period of time. Controlled release may be achieved through use of encapsulation methods discussed above. Controlled release may alternatively, or additionally, be achieved through selection of materials that are configured as “fast release” and “slow release” forms of the material. Further, controlled release configurations may be applied to any material, with any product, and/or with any use of the hollow core granules as otherwise described herein. While certain products discussed herein may be specifically described in relation to controlled release forms thereof, it is understood that these controlled release properties may be applied to any product described herein regardless of whether such feature is specifically called out in relation to the separate discussion of the product herein.

In some embodiments, a wall of a hollow core structure as described herein may be formed from a gelled material. Such gelled materials may comprise at least one hydrophilic long-chain polymer and at least a water source. Hydrophilic, long-chain polymers useful herein can include long chain carbohydrates (e.g., polysaccharides) as well as various proteins. The hydrophilic, long-chain polymer preferably is configured to thicken and form a gel upon hydration (with or without heating). Non-limiting examples of hydrophilic, long-chain polymers that may be utilized in forming a wall according to the present disclosure can include: gelatin, pectin, carrageenan, gellan gum, guar gum, locust bean gum, gum arabic, xanthan gum, starch, methylcellulose, agar, konjac, alginates, and combinations thereof (including single, binary, tertiary, or quaternary blends). The hydrophilic, long-chain polymer may comprise about 0.1% to about 20%, about 1% to about 15%, or about 2% to about 10% by weight of the gelled material used to form the wall of the hollow core structure. The gelled material may otherwise comprise about 80% to about 99.9%, about 85% to about 99%, or about 90% to about 98% by weight of a water source, particularly deionized water.

In some embodiments, a wall of a hollow core granule may comprise a lipidic material. Non-limiting examples of lipidic bases include oils, fats, and compositions formed therewith. In some embodiments, edible fats in particular may be used. Suitable lipidic materials for use in forming lipophilic compositions include fats and oils derived from one or more of a vegetable source, an animal source, a nut source, a seed source, and the like. Suitable lipidic material may be predominately or completely saturated, predominately or completely unsaturated, or hydrogenated. Non-limiting examples of suitable lipidic materials include fats and/or oils derived from one or more of the following: cocoa, palm, coconuts, almonds, cashews, hazelnuts, macadamia nuts, peanuts, pecans, pistachios, walnuts, pumpkin seeds, sesame seeds, soybeans, rapeseed, corn, safflower seeds, and the like. Specific, non-limiting examples of lipid based materials that may be used in preparing a composition as described herein include chocolates with any cocoa concentration (e.g., milk chocolate, dark chocolate, white chocolate), palm fat, coconut fat, peanut butter, hazelnut fats, vegetable oils, milk fats, confectionary fats (such as available from AAK, AB), and the like. Such materials may include additional components, such as sugar, salt, other oils, and the like. For example, chocolates may comprise sugar, cocoa butter, cocoa processed with alkali, milk fat, lactose (e.g., from milk), soy lecithin, emulsifier, vanillin, artificial flavor, milk, and/or other ingredients. Dairy components utilized in lipophilic compositions can include fats, proteins, and/or sugars derived from cow milk, goat milk, and the like.

As noted above, in one or more embodiments, a wall of a hollow core granule will be prepared though use of a binder, and the formed wall will retain a content of the binder material. In some embodiments, however, substantially all of the binder may be removed from the structure during processing of the structure. This may occur in particular when one or more wall forming materials as described above is of a nature whereby the particles thereof may remain bound even after removal of the binder material. In particular embodiments, at least a portion of the binder will be retained in the wall of the formed hollow core granules. For example, formed granules may include binder retained in the wall of the granule (e.g., in at least a portion of interstitial spaces between individual particles of the wall forming material) in an amount of about 0.1% to about 50% by weight, about 1% to about 45% by weight, about 2% to about 40% by weight, or about 5% to about 30% by weight, based on the total weight of the granules. The remaining weight of the granules may be accounted for by the wall forming material(s) alone or in combination with any coatings applied to the granules.

Binder materials particularly may be provided in particular formats for use in processing to form the hollow core granules. Specifically, it can be beneficial for the binders to be in particulate form when added to the processing equipment. In this manner, the particles of the wall forming material can agglomerate or accumulate around the particles of the binder as the solid binder softens with heating. Thereafter, as the binder liquefies, the liquid binder will flow out of the core of the forming granules and into the walls forming with the wall forming materials. To this end, it can be particularly useful for the binder particles, seeds, or crystals to have a starting size in a range of about 0.1 mm to about 5 mm, about 0.5 mm to about 4 mm, or about 0.8 mm to about 3 mm.

A variety of materials may be utilized as a binder. In some embodiments, a binder may be a material that is substantially solid at a temperature of about 50° C. or less, about 45° C. or less, or about 40° C. or less and is liquid above such temperature. In certain embodiments, the binder may be adapted to or configured to be a solid at a temperature in the range of about 10° C. to about 50° C., about 15° C. to about 45° C., or about 20° C. to about 40° C. Additionally, or alternatively, the binder may be a material with a melting point in the range of about 40° C. to about 95° C., about 45° C. to about 90° C., or about 50° C. to about 90° C. The binder may also be chosen for a defined application, as further described herein, based upon whether the binder is hydrophilic or hydrophobic. For example, in some embodiments, hydrophobic binders may be utilized, such as paraffinic hydrocarbons, olefinic hydrocarbons, waxes, beeswax, or similar materials exhibiting the foregoing state change characteristics. Hydrophobic polymers may likewise be utilized. Non-limiting examples of suitable, hydrophobic binders can include wax, paraffin, polycaprolactone, ethylene-vinyl acetate copolymers, polypropylene carbonate, poly(tetramethylene oxide), poly(ethylene adipate), poly(trans-butadiene), thermoplastic polyurethane (e.g., carbothane TPU), stearic acid, and the like. Likewise, one or more lipidic materials described above may be utilized as a hydrophobic binder. In further embodiments, the binder may specifically be a hydrophilic material, such as a polyethylene glycol (PEG). Further examples of suitable binders include materials such as polyoxyethylene fatty ethers derived from various types of alcohols (e.g., lauryl, cetyl, stearyl, and oleyl alcohols), and such materials are available under names such as Brij™ S100 (polyoxyethylene stearyl ether) or Steareth-100. Such polyoxyethylene fatty ethers may be useful as a hydrophilic binder although being more hydrophobic in nature that other hydrophilic binders, such as PEG materials. Likewise, fatty acids with carbon chain lengths in the range of C10 to C30 may be useful as binders, one example embodiment being stearic acid. In one or more embodiments, binders may be a material having a melting temperature this is less than a melting temperature of the material used in forming the wall of the hollow core structure. As such, suitable binder materials may have a substantially high melting temperature, such as in the range of about 90° C. to about 200° C., about 100° C. to about 180° C., or about 110° C. to about 160° C. For example, plastics (e.g., polyvinylchloride (PVC), high density polyethylene (HDPE), etc.), thermoplastics, rubbers, and similar materials may be utilized as binders in some embodiments.

In some embodiments, binders may be chosen specifically in relation to the viscosity of the binder in the liquefied form. Binders with lower liquid viscosities can achieve faster processing for granule formation while binders with higher liquid viscosities can result in longer processing requirements for granule formation. Likewise, however, binder liquid viscosity can affect one or more properties of the finished granules. For example, binders with higher liquid viscosities can lead to relatively stronger granules. As such, binder choice can be a factor of binder liquid viscosity. In some embodiments, flow properties of the binder in liquid form may be controlled, at least in part, through selection of binder molecular weight. For example, PEG materials can be particularly useful as binders, and various grades so PEG materials can be chosen based at least in part on the molecular weight of the material. In various embodiments, suitable PEG materials for use as a binder in the hollow core granules particularly can have a molecular weight of at least 400 Da, at least 1000 Da, at least 2000 Da, or at least 4000 Da. Maximum molecular weight can be, for example, no greater than 50000 Da, no greater than 45000 Da, or no greater than 40000 Da. More particularly, PEG molecular weight can be in the range of about 400 Da to about 34,000 Da. In specific embodiments, a lower range, such as about 400 Da to about 15000 Da, about 500 Da to about 12000 Da, or about 1000 Da to about 10000 Da may be used. In other embodiments, a high range, such as about 8000 Da to about 34000 Da, about 10000 Da to about 30000 Da, or about 12000 Da to about 25000 Da may be used.

Molecular weight can be expressed as a weight average molecular weight (M_{w}) or a number average molecular weight (Mₙ). Both expressions are based upon the characterization of macromolecular solute containing solution as having an average number of molecules (nᵢ) and a molar mass for each molecule (Mᵢ). Accordingly, number average molecular weight is defined by formula 1 below.

$${M}_{n} = \frac{\sum {n}_{i} {M}_{i}}{\sum {n}_{i}} \qquad ( 1 )$$

Weight average molecular weight (also known as molecular weight average) is directly measurable using light scattering methods and is defined by formula 2 below.

$${M}_{w} = \frac{\sum {n}_{i} {M}_{i}^{2}}{\sum {n}_{i} {M}_{i}} \qquad ( 2 )$$

Molecular weight can also be expressed as a Z-average molar weight (M_{z}), wherein the calculation places greater emphasis on molecules with large molar weights. Z-average molar weight is defined by formula 3 below.

$${M}_{z} = \frac{\sum {n}_{i} {M}_{i}^{3}}{\sum {n}_{i} {M}_{i}^{2}} \qquad ( 3 )$$

Unless otherwise noted, molecular weight (MW) is expressed herein as weight average molecular weight.

Although a variety of solid, wall-forming materials are described above along with a variety of binders, it is understood that the present disclosure contemplates all combinations of wall-forming materials and binders as described herein and as would be otherwise recognized as useful in light of the present disclosure. As such, the present disclosure encompasses hollow core structures wherein at least one wall or layer comprises any of the following: particles of one or more types of clay (e.g., bentonite) combined with at least one binder described above; particles of glass combined with at least one binder described above; particles of one or more ceramics combined with at least one binder described above; particles of one or more aluminas combined with at least one binder described above; particles of one or more silicates combined with at least one binder described above; particles of one or more zeolites combined with at least one binder described above; particles of carbon combined with at least one binder described above; particles of one or more metals combined with at least one binder described above; particles of one or more salts (e.g., sodium bicarbonate or baking soda, sodium carbonate or soda ash, or sodium chloride) combined with at least one binder described above; particles of one or more cleaning compositions combined with at least one binder described above; particles of one or more fertilizers combined with at least one binder described above; particles of one or more pesticides combined with at least one binder described above; particles of one or more absorbents and/or adsorbents combined with at least one binder described above; particles of one or more deodorizers and/or odor control agents combined with at least one binder described above; particles of one or more bleaches or bleaching agents combined with at least one binder described above; particles of one or more oxidizers combined with at least one binder described above; particles of one or more reducers combined with at least one binder described above; particles of one or more gellants combined with at least one binder described above; particles of one or more fillers combined with at least one binder described above; and particles of one or more chelators combined with at least one binder described above. Of course, it is understood that any type of material as described herein may be used as a wall forming material alone or in combination with a solid where the desired material is not in a solid form.

As further discussed herein, choice of wall forming material and/or choice of binder material can be effective to customize the hollow core granules to exhibit various properties. In some embodiments, the hollow core granules may be defined in relation to water absorption capacity. This can be a characterizing feature particularly in relation to hollow core granules including appropriate wall forming materials and/or binders so that the hollow core granules are hydrophilic. In example embodiments, hollow core granules can have a water absorption capacity such that the hollow core granules will absorb a weight of water that is about 5% to about 80%, about 10% to about 70%, or about 15% to about 60% of the initial weight of the hollow core granules. The hollow core granules likewise may exhibit greater water absorption than the wall forming material alone. For example, the hollow core granules may have a water absorption that exceeds the water absorption of the wall forming material used in forming the hollow core granules (i.e., when the wall forming material is in its native form, prior to incorporation into the hollow core granules) by an amount of about 2% to about 20%, about 2% to about 15%, or about 3% to about 10%.

In some embodiments, the hollow core granules may be defined in relation to oil absorption capacity. This can be a characterizing feature particularly in relation to hollow core granules including appropriate wall forming materials and/or binders so that the hollow core granules are hydrophobic. In example embodiments, hollow core granules can have an oil absorption capacity such that the hollow core granules will absorb a weight of oil that is about 5% to about 80%, about 10% to about 70%, or about 25% to about 65% of the initial weight of the hollow core granules. The hollow core granules likewise may exhibit greater oil absorption than the wall forming material alone. For example, the hollow core granules may have an oil absorption that exceeds the oil absorption of the wall forming material used in forming the hollow core granules (i.e., when the wall forming material is in its native form, prior to incorporation into the hollow core granules) by an amount of about 5% to about 50%, about 10% to about 40%, or about 15% to about 35%.

### Methods of Preparation

Hollow core structures according to the present disclosure may be prepared according to a variety of methods. In one or more embodiments, a method for preparing a structure with a substantially hollow core may comprise combining a binder a described herein with a plurality of solid particles of a wall-forming material as described herein to form a mixture. The wall-forming material may particularly be a material that is substantially insoluble in the binder and that has a melting point that is greater than the melting point of the binder. In light of the example embodiments of solid, wall forming materials and the example embodiments of binders provided above, it will be readily evident which types of solid, wall forming materials may be combined with which types of binders to carry out such methods. In example embodiments, a suitable binder may be a material having a melting point of about 40° C. to about 95° C. (or a further range as described above), and suitable solid particles may be a material that has a melting point of about 60° C. or grater, about 70° C. or greater, about 80° C. or greater, about 100° C. or greater or about 110° C. or greater. Of course, it is understood that a suitable binder may be chosen so that the binder has a melting point that is less than the melting point of the wall forming material by at least 5° C., at least 10° C., at least 15° C., or at least 20° C. The binder and solid particles may be combined at a temperature that is below the melting point of the binder, such as at room temperature or ambient temperature. The binder and the solid particles may be mixed at this temperature for a certain time, such as about 15 seconds to about 180 seconds, about 30 seconds to about 150 seconds, or about 45 seconds to about 120 seconds to provide a substantially uniform mixture.

Combination of the materials may be in a first container for transfer to a second container for heating. Alternatively, the process can be carried out in a single unit, such as a fluidized bed reactor. As such, a fluidizing gas, such as air, may flow upward through the bed to provide mixing and, optionally, to provide for heating and/or cooling of the mixture. Other types of reactors may also be used. When using a fluidized bed reactor, the particles of binder material may be first added to the fluidized bed followed by the particles of the wall forming material.

The mixture of the binder and the solid particles can be heated to a maximum temperature to cause melting of the binder. As such, the maximum temperature can be a temperature that is above the melting point of the binder and below the melting point of the plurality of solid particles. Such heating can be adapted to or configured to form agglomerations of the solid particles. In some embodiments, the maximum temperature may be a temperature that exceeds the melting point of the binder by about 5° C. or more, about 10° C. or more, or about 20° C. or more. The binder may alternatively, or additionally, be at least partially fluidized (e.g., melted) when added to the wall forming material. For example, binder in a liquid form may be sprayed onto particles of the wall forming material, such as through an atomizer or similar unit adapted to or configured to provide the liquid binder in a substantially fine, spray or mist form. In some embodiments, in situ melting can be utilized where binder particles that are substantially larger than the particles of the wall-forming material are utilized. Specifically, hollow core particles may be formed by immersion of the particles of the wall-forming material into molten binder particles and subsequent layering. Preferably, however, materials will be supplied in a suitable configuration so that particles of the wall forming material with accumulate or agglomerate around seed particles or crystals of the binder so that, as heating continues, the binder will flow out of the center of the forming granules and into the interstitial spaces of the particles forming the wall.

In some embodiments, heating can be carried out utilizing a specified heating rate. For example, it can be desirable for heating to be carried out at a rate of about 5° C. per minute to about 25° C. per minute, about 7° C. per minute to about 22° C. per minute, or about 10° C. per minute to about 20° C. per minute. Heating may begin at ambient temperature, the heating may be applied at the noted rate until the maximum temperature is reached. In some embodiments, the maximum temperature may be maintained for a defined period of time. For example, the maximum temperature may be maintained for a time of about 30 seconds to about 1 hour, about 30 seconds to about 45 minutes, or about 2 minutes to about 30 minutes. As seen in the appended examples, residence time at the maximum heating temperature can affect the final granule properties, including wall thickness, granule size, and percentage of binder present in the wall of the formed granule.

In some embodiments, processing time in a fluidized bed reactor can be controlled to adjust the average size of individual granules of the hollow core structures that are prepared. Processing time may also be adjusted to control other properties, such as the size of the cavity in individual granules of the hollow core structures, the ratio of cavity diameter to overall granule diameter, and the bulk density of the granules. In some embodiments, processing time in a fluidized bed reactor can be adjusted to be within a range of about 10 minutes to about 20 minutes or a range of about 12 minutes to about 18 minutes in order to maximize one or more the noted properties. Lesser processing times (e.g., about 1 minute to about 9 minutes or about 3 minutes to about 7 minutes) and/or greater processing times (e.g., about 22 minutes to about 30 minutes) can be utilized to provide lower values. Processing times may likewise be adjusted based upon the viscosity of the liquefied binder. Specifically, higher viscosities may require longer residence times while lower viscosities may require lesser residence times.

The formed agglomerations of the plurality of solid particles can be cooled to provide the plurality of granules that each have a substantially hollow core (i.e., an internal cavity). In particular, this can include cooling to a temperature that is below the melting point of the binder. In some embodiments, it can be beneficial to effect a substantially rapid cooling of the solid particles, such as cooling to below the melting point of the binder within a time of about 5 seconds to about 5 minutes, about 10 seconds to about 3 minutes, or about 15 seconds to about 2 minutes. In other embodiments, longer cooling times may be utilized, such as about 5 minutes to about 60 minutes, about 10 minutes to about 50 minutes, about 20 minutes to about 40 minutes, or about 25 minutes to about 35 minutes.

As a non-limiting example, in some embodiments, preparation of structures described herein may be carried out in a multi-stage mixer. For example, in a first stage mixer, particles of the wall forming material may be combined with a binder and effectively form a relatively thin coating of the wall forming particles around a crystal or particle(s) of the binder. The mixing may continue while particles of the wall forming continue to flocculate around the binder or otherwise combine to increase the thickness of the wall. If desired, particles from the first stage mixer may be passed to a second stage mixer where flocculation or particle adherence may continue for formation of the wall. Structures having a desired wall thickness may then be passed to a rotary dryer (or similar structure) for removal of a portion or substantially all of the binder from the structures to leave the structures with the desired, hollow core configuration. Because of such flocculation, choice of binder material can be used to adjust the size of the cavity within individual granules of the hollow core structures. Binders that tend to exist as relatively smaller particles or crystals can thus be chosen to form individual granules with relatively small core diameters, and binders that tend to exist as relatively larger particles or crystals can thus be chosen to form individual granules with relatively large core diameters.

As discussed above, while particles of the wall forming material may initially flocculate around particles of the binder material, as the binder material liquefies, the binder material can flow out from the core of the forming granule and into accumulating particles of the wall forming material. The evacuation of the binder from the interior of the forming and/or formed granules results in the interior cavity of the granule. A portion of the binder may remain at one or both of the inner wall surface 19 and the outer wall surface 17 of the wall forming the individual granule. Likewise, a portion of the binder may remain in the interstitial spaces 154 as discussed previously. As non-limiting examples, a formed structure with a substantially hollow core according to the present disclosure may be configured so that the amount of binder present in the wall of the hollow core structure is about 0.1% to about 50% by weight, about 1% to about 45% by weight, about 2% to about 40% by weight, or about 5% to about 30% by weight based on the total weight of the granules.

In one or more embodiments, a structure with a substantially hollow core may be prepared in a gel-forming process. Such process can be particularly useful for forming hollow core structures with a substantially continuous phase outer wall that is a gel or hydrogel and comprising mainly water and a gel-former. Such hollow core structures may be utilized as formed or may be further treated, such as to form a further, outer wall surrounding the gel wall.

Methods for preparing structures according to such embodiments can comprise providing a solution of a gel-former in water. The gel-former particularly can be a hydrophilic long chain polymer as otherwise described herein. Preferably, the gel-former and water can be at an increased temperature or can be specifically heated to such temperature to expedite polymer dissolution to form a solution. For example, the solution of gel-former in water can be at a temperature of about 50° C. or greater, about 60° C. or greater, or about 70° C. or greater, such as about 50° C. to about 95° C., about 55° C. to about 90° C., or about 60° C. to about 85° C. The solution may be stirred or simply left at the increased temperature until substantially all of the gel-formed has dissolved—e.g., as evidenced by visual inspection.

The methods further can comprise contacting a stream of the solution with a hydrophobic liquid in a manner adapted to or configured to form droplets of the gel-former (e.g., the hydrophilic long chain polymer). The contacting can be via a variety of means. For example, a stream of the solution and a stream of the hydrophobic liquid can be simultaneously poured such that the two streams may sufficient physical contact to cause the solution to separate into the gel droplets. In some embodiments, the hydrophobic liquid can be provided a container, and the solution of the gel-former in water can be poured or otherwise introduced into the container. If desired, the solution may be delivered for contact with the hydrophobic liquid in a substantially droplet form or relatively thin stream form. For example, the solution may be delivered through a syringe pump or similar device including one or a plurality of outlets of a desirably small size, such as a diameter of about 0.01 mm to about 2 mm, about 0.05 mm to about 1.5 mm, about 0.1 mm to about 1.2 mm, or about 0.2 mm to about 1 mm.

The solution may be at least partially cooled prior to combining with the hydrophobic liquid and/or may be cooled by contact with the hydrophobic liquid. In some embodiments, pre-cooling may be excluded. Preferably, the hydrophobic liquid is at a temperature that is less than the temperature of the solution of the gel-former. For example, the hydrophobic liquid may be at a temperature of about 45° C. or less, about 40° C. or less, or about 35° C. or less (e.g., about 5° C. to about 40° C., about 5° C. to about 25° C., or about 5° C. to about 20° C. In some embodiments, the hydrophobic liquid may be provided in a refrigerated tank or similar storage unit.

Optionally, the method may comprise separating the gel droplets from the hydrophobic liquid. When two streams of the materials are simultaneously contacted, the separation may occur during the forming step—e.g., by combining the streams over an appropriately sized sieve or the like so that the gel droplets are captured. Alternatively, when the stream of the solution is added to a hydrophobic liquid in a container, the mixture of hydrophobic liquid and formed gel droplets may be processed through an appropriately sized sieve or the like to capture the gel droplets. In some embodiments, a conveyer or similar transport system can be used for collected gel droplets (or beads) to be moved out from the hydrophobic liquid tank.

In some embodiments, it can be useful to carry out washing of the gel droplets, such as with a soap, to provide substantially cleaned gel droplets. This may be achieved, for example, by rinsing with a soap solution, temporarily soaking the gel droplets in a soap solution bath followed by rinsing with substantially pure water, or any similar method. This can be beneficial since residual hydrophobic liquid on the gel droplets can render the gel droplets substantially hydrophobic and reduce final strength and water absorption properties of the gel droplets. Washing with a soap or the like can thus provide substantially cleaned gel droplets.

It can further be useful to at least partially coat the substantially cleaned gel droplets with a conditioning agent to form conditioned gel droplets. The conditioning agent can be any material or combination of materials that are adapted to or configured to substantially prevent the gel droplets from adhering to one another. As such, the conditioning agent may function as a flow aid. Further, the conditioning agent may be material or materials useful to improve the adherence of a coating layer/wall onto the gel droplets. In some embodiments, the conditioning agent may be a mixture of an inert powder and an oil. For example, talcum powder, powdered starch(es) (e.g., cornstarch, tapioca starch, arrowroot starch, rice starch), grain flour(s) (e.g., oat flour), fumed silica, precipitated silica, confectionary sugar, calcium silicate, sodium aluminosilicate, sodium ferrocyanide, potassium ferrocyanide, calcium ferrocyanide, calcium carbonate, magnesium carbonate, cellulose powder, bone phosphate, sodium silicate, silicon dioxide, magnesium trisilicate, potassium aluminum silicate, bentonite, aluminum silicate, steric acid, polydimethylsiloxane, and the like may be utilized as an inert powder. Suitable oils can include silicone oil, mineral oil, dimethicone, and the like.

Addition of the conditioning agent may be particularly useful in relation to the later addition of a coating layer on the gel droplet. For example, a clay material in particular may be formed into a coating layer, and this can include contacting the conditioned gel droplets with clay particles or powdered clay (or other materials as already described herein). When a coating layer it applied to the gel droplets, in can be useful to carry out a drying step. For example, the gel droplets with the coating layer may be dried at ambient temperature or increased temperature and alternatively, forced air drying may be utilized. In some embodiments, the gel droplets with the coating layer may be dried at a temperature of about 90° C. or greater, about 100° C. or greater, or about 110° C. or greater (e.g., about 90° C. to about 150° C., about 100° C. to about 140° C., or about 110° C. to about 130° C.). Preferably, drying at increased temperature may be carried out after coating is completed. Coating may be carried out using a variety of coating units, such as a plate granulator, drum granulator, or the like where the gel droplets may be substantially uniformly intermixed with the coating material.

In one or more embodiments, the present disclosure thus can provide for a substantially continuous process for manufacturing hollow core structures. Such process can include forming hydrogel beads/droplets, washing the formed hydrogel beads/droplets, and coating the hydrogel beads/droplets utilizing a powdered or particulate, solid coating material. More particularly, forming the bead/droplets can include contacting a hydrogel solution with a hydrophobic liquid that is optionally refrigerated, and this can include delivering the hydrogel solution from a storage container through a syringe pump or similar component that can include a plurality of outlets. The beads/droplets may form substantially spontaneously in the hydrophobic liquid, and them may be removed therefrom via a conveyor system or similar unit to a washing/rinsing stage. In the washing/rinsing stage, the beads/droplets may have a residual layer of the hydrophobic liquid that may be substantially or completely removed therefrom, such as through contact with a detergent solution that may be sprayed or otherwise contacted with the beads/droplets. The washed/rinsed beads/droplets optionally may be at least partially dried, such as through passage through a heater and/or air dryer. The washed/rinsed beads/droplets that are optionally at least partially dried may optionally be pre-conditioned as discussed above. As such, the beads/droplets may be spray coated or otherwise contacted with a suitable conditioning material. The beads/droplets having undergone washing/rinsing and any further, optional treatments, may then be passed through a coating unit, which may consist of one or a plurality of mixing stages wherein the beads/droplets are contacted with the powdered or particulate, solid coating material until reaching a desired coating thickness. The thus-coated beads/droplets can then be passed through a drying unit for drying by heat and/or forced air. The dried beads/droplets may be ready for use or may optionally be passed through one or more further mixing units for addition of further coating layers, such as further conditioning layers and/or further layers of the coating material (e.g., a bentonite powder or other coating material as described herein). Such process may be substantially continuous in that the beads/droplets may be continuously formed and transported along a conveying system or similar, suitable system, from one processing unit to the next to provide the finished, hollow core structures.

### Products and Articles of Manufacture

The hollow core structures/granules can be utilized in forming a variety of products. Such products may be defined in relation to a functional aspect thereof and/or in relation to a physical property thereof that arises at least in part from the configuration of at least one component of the product as a hollow core structure as described herein. The methods of manufacture described above make it possible to configure a variety of solid materials (e.g., compounds, minerals, and mixtures of multiple components) into a hollow core form that can result in improved properties as compared to the same material when provided in its dense form (i.e., not having an internal cavity or hollow core). For example, provision of a material in a hollow core form as described herein can provide for increased uses and improved performance, such as reducing material weight or bulk density, improving product solubility, improving absorptive and/or adsorptive properties, improving release of a component, improving flowability of solid granules, or similar properties, and the like. In relation to mixtures of different materials, a single component of the mixture may be provided in the hollow core form and thus impart improved properties to the overall mixture of materials. Likewise, a plurality or even all of the components of a mixture may be provided in the hollow core form. For example, a mixture may include one or more components separately configured as hollow core granules (e.g., a first group of hollow core granules where a first component is the wall forming material and a second group of hollow core granules where a second component is the wall forming material, and optionally more groups of hollow core granules, the groups of hollow core granules being admixed). As another example, a mixture may include one or more components combined as hollow core granules (e.g., a group of hollow core granules where all of two or more components are used as the wall forming material). As a further example, a mixture may include any of the foregoing types of hollow core granules and one or more components not in the hollow core granule form.

In some embodiments, products provided in the form of a hollow core structure can exhibit improved solubility versus the non-hollow core version of the material. The improved solubilization can be particularly pronounced when the materials are compared on a size basis. A granule prepared as a hollow core structure with an outer wall comprising a plurality of individual particles of a given material is significantly larger in size than the individual particles of the material that are present in the outer wall of the granule. The larger granule can be configured to readily break apart in the presence of a suitable solvent so that the smaller particles forming the wall of the granule will individually solubilize. A fully dense particle of the material present in substantially the same size as the hollow core granule will dissolve significantly slower as the solvent slowly penetrates through the surface. Thus, the granule formed of a wall of individual particles of the material will exhibit significantly greater surface area for interaction with the solvent. Likewise, the binder utilized in forming the granule can be chosen for solubility in the desired solvent. For example, in relation to solid materials that are intended for dissolution in aqueous solvents or polar solvents, hydrophilic binders, such as various PEG materials, may be utilized, and the binder will this participate at least in part with rapid dissolution of the granule in the solvent. Similarly, in relation to solid materials that are intended for dissolution in non-polar solvents, hydrophobic binders, such as waxes or hydrophobic polymers, may be utilized, and the binder again will participate at least in part with rapid dissolution of the granule in the solvent. In some embodiments, a time to substantially complete solubilization of a specified weight of granules having a hollow core structure as described herein can be at least 10%, at least 25%, at least 50%, or at least 75% faster than a time to substantially complete solubilization of the same weight of the same material in a fully dense form (i.e., not in a hollow core form). More particularly, the hollow core form of the material may be substantially solubilized at a rate that is about 10% to about 99%, about 15% to about 95%, about 20% to about 90%, or about 25% to about 80% faster than the non-hollow core form of the same material.

The nature of the hollow core structures described herein as being formed from a plurality of particles of one or more materials that are bound in a wall with a binder material can provide a variety of options for controlled release compositions. Different materials will have different dissolution rates in various solvents and solvent temperatures due to the chemical and/or physical nature of the materials. Based upon identified dissolution rates of the materials, it is possible according to the present disclosure to provide granules of hollow core structures wherein the wall thereof comprises particles of two or more different materials with two or more different dissolution rates. For example, as further discussed herein, the present hollow core structures may be utilized in fertilizer products. Various chemicals and compounds useful as fertilizers can exhibit different dissolution rate or release rates. In particular, there are variously know “fast release” fertilizers and “slow release” fertilizers. Where it is desirable to provide combinations of fertilizers with different release rates, particles of a fast release fertilizer and particles of a slow release fertilizer can be combined in desired ratios and used as the wall forming component for preparing granules of hollow core structures as described above. Thus, the resulting fertilizer granules will have a wall surrounding a hollow core wherein the wall comprises the fast release fertilizer particles and the slow release fertilizer particles in the designed ratio. Upon application to a site in need of fertilization, the fast release fertilizer particles will provide immediate fertilization, and the slow release fertilizer particles will remain for the expected time for slow release thereof. The same principle can apply to any number of solid materials with differing dissolution and/or release rates so that many types of controlled release granules can be prepared.

Similarly, controlled release may be achieved by using two or more different forms of the same solid material. For example, a desired material may be provided as particles in two or more different forms that thus exhibit two or more different dissolution or release rates. The different release rates may relate to particle size, particle purity, presence of an encapsulating layer, or other recognized manners for affecting dissolution or release rate. For example, first particles of a first size may have a first dissolution or release rate with second particles of a second, different size may have a second, different dissolution or release rate. As a further example, a first set of particles that are substantially pure (i.e., formed entirely of a single material or having only a minor amount of impurities) may exhibit a first dissolution or release rate, and a second set of particles may include an additive (e.g., an inert material or a different, desired material that has a different dissolution or release rate) in an amount that causes the second set of particles to have a dissolution or release rate that differs from the first set of particles. As yet a further example, a first set of particles may be provided in an uncoated state, and a second set of particles of the same material may be provided with a coating or in an encapsulated form so that the coated or encapsulated particles exhibit a delayed release relative to the uncoated or non-encapsulated particles. These or similar situations can be applied to two, three, four, or even more sets of particles that then can be mixed in desired ratios and used as the wall forming material to prepare granules wherein the wall surrounding the hollow core comprises two, three, four, or even more sets of particles with two, three, four, or even more, different dissolution or release rates. For example, in a laundry care application, it may be desirable to provide for immediate release of detergent materials in the wash liquor but have a delayed release of bleaching materials, brighteners, or the like. In such cases, the laundry components for immediate release may be provided in an unmodified form, and the laundry components for delated release may be provided in an encapsulated or coated form, and the different materials may then be mixed and used as the wall forming material to prepare granules of the laundry cleaning composition that, when added to the wash liquor, will immediately release the detergent components therefrom while delaying release of the further components (i.e., the coated or encapsulated components).

In some embodiments, the provision of materials in a hollow core form as described herein can be particularly beneficial in providing for reduced product weight without limiting product performance. For example, solid, particulate products that are often sold in large quantities may exhibit an undesirably high weight that can be troublesome for consumers to carry and manipulate. By providing such products in a hollow core form, the total weight can be reduced while still providing the product in a volume effective to achieve the desired end result and thus avoid an effective increase in cost to the consumer in order to achieve the same result. In other words, the effective volume of the product can still provide substantially the same end result at approximately the same product cost but with a reduced product weight.

In example embodiments, such desired reduction in overall weight can particularly applied in the field of animal litters, which are often formed at least in part from dense products, such as clays. Clays are often used in animal litters because they are a relatively inexpensive and effective liquid absorbing material. Clays, however, are relatively dense and cause animal litter products to be quite heavy with commercially sold quantities requiring as much as 30 to 40 pounds of a clay-based litter to fill a large-size litter tray. The ability to provide hollow core structures as described herein thus can be particularly useful in forming an animal litter having a significantly lower weight and even improved absorption properties. This can extend to clay-based hollow core structures as well as non-clay hollow-core structures.

A reduction in weight or mass of a given material by providing the material in a hollow core form as described herein can vary based upon the density of the substantially pure product. Higher density materials will exhibit a greater reduction in product mass or weight when provided in a hollow core form relative to materials with lesser densities. In some embodiments, a specified volume of a material provided in a hollow core form according to the present disclosure can have a mass or weight that is at least 5%, at least 10%, at least 15%, or at least 20% less than the mass or weight of the same volume of the material when provided in its native or typical non-hollow core form. In certain embodiments, the hollow core version can have a mass or weight that is about 5% to about 60%, about 7% to about 40%, or about 10% to about 35% less than the mass or weight of the same volume of the non-hollow core version of the product.

In some embodiments, granules formed as hollow core structures can exhibit improved ability to absorb and or adsorb gases and liquids. As such, materials that are previously known to exhibit good absorption and/or adsorption properties in their typical, dense form can have such properties improved by configuring particles of the material as a wall around a hollow core. Likewise, materials that do not necessarily exhibit absorption and/or adsorption properties in their typical, dense form can be utilized for such purposes when particles of the material are configured as a wall around a hollow core. While not wishing to be bound by theory, it is believed that improvements in absorption and/or adsorption properties can arise at least in part due to the increased porosity achieved through binding together a significantly large number of smaller particles of the material into a wall surrounding a hollow core. Likewise, the combination of a large number of small particles in a shell structure can significantly increase available surface area to absorption and/or adsorption purposes. Still further, the addition of a binder in the shell structure can likewise provide absorption and/or adsorption properties that are additive with such properties existing in the particles of the solid, wall forming material itself. Such properties can extend to uses in odor absorption (i.e., uptake of odor causing chemicals that may exist in a substantially gaseous state) as well as liquid absorption (e.g., spill clean-up).

Improved absorption and/or adsorption particularly can indicate that the same volume or weight of a gas or liquid may be absorbed by a lesser weight of the hollow core granules relative to the same material in its native or typical, fully dense form (i.e., not in a hollow core form). For example, the present hollow core structures may provide for at least 10%, at least 25%, at least 50%, or at least 75% greater absorbance of a gas and/or a liquid (based on the volume of a gas or based upon either of the volume or the mass of a liquid) compared to the same weight of the material when not in the hollow core form as presently described. More particularly, such improvement may be in the range of about 10% to about 95%, about 15% to about 90%, about 20% to about 85%, or about 25% to about 75%.

In example embodiments, hollow core granules with improved absorption and/or adsorption of gases and that are configured to be functional as a deodorizer (i.e., configured to absorb, adsorb, or otherwise trap, bind, and/or neutralize odor causing compounds) can be prepared with a variety of wall forming materials, a variety of binders, and can include optional odor neutralizing agents. For instance, various clays (e.g., bentonite), salts (e.g., sodium bicarbonate), carbon materials (e.g., activated carbon), and high porosity materials (e.g., zeolites) can be effective at capturing odor causing compounds, and any one or more of such materials, or other materials exhibiting similar efficacy, may be used as a wall forming material for the hollow core granules. Suitable binders can include materials such as PEGs of various molecular weights (e.g., PEG 8000, PEG 12000, and/or PEG 35000), saturated fatty acids (e.g., stearic acid), and polyoxyethylene fatty ethers (e.g., Brij™ S100). Odor neutralizing agents may be provided as solid that may be included as a wall forming material, may be liquids that are combined with solid, wall forming materials, may be liquids that are blended with the binder, or may be included in the hollow core granules in any other suitable manner. One example of a suitable odor neutralizing agent is lauryl methacrylate. Odor masking agents may likewise be utilized and can encompass fragrances and the like that can deliver a desired odor in an amount sufficient to mask an undesired odor.

The improved ability of a hollow core granule as described herein to mitigate malodor by absorbing, adsorbing, or other binding odor-causing chemicals or compounds is illustrated in Example 12 herein. Specifically, it has been shown that when a material effective as an odor controlling agent is used as a wall forming material in a hollow core granule according to the present disclosure, the hollow core form of the material will exhibit improved functionality versus the same material in its native state. For example, a hollow core form of an odor reducing agent can show improved malodor reduction versus the native form of the odor reducing agent in that a detectable concentration of an odor causing chemical or compound can be at least 10% less, at least 25% less, at least 50% less, at least 75% less, or at least 90% less after a defined time of contact of the odor causing chemical or compound with the odor reducing agent. Testing in Example 12 showed a continuing ability for the hollow core form of the odor reducing agent to provide improved malodor reduction that increased with time. As such, the relevant time period for the above ranges may be as little as 1 hour or even as long as 100 hours.

In further example embodiments, hollow core granules with improved absorption and/or adsorption of liquids and that accordingly can be configured to be functional for spill cleanup or similar uses can be prepared with a variety of wall forming materials, a variety of binders, and can include optional additives for achieving defined purposes, such as degradation of organics. For instance, various clays (e.g., bentonite), carbon materials (e.g., activated carbon), and high porosity materials (e.g., zeolites) can be effective for absorbing aqueous and/or non-aqueous liquids at various locations (terrestrial and/or water), and any one or more of such materials, or other materials exhibiting similar efficacy, may be used as a wall forming material for the hollow core granules. Suitable binders can be chosen based upon the desired use. For example, in certain embodiments, binders can be chosen specifically to prepare granules for terrestrial use in absorption of liquids, including hydrocarbons, such as various oils. Suitable binders for such purposes can include hydrophilic materials such as PEGs of various molecular weights (e.g., PEG 8000, PEG 12000, and/or PEG 35000), saturated fatty acids (e.g., stearic acid), and polyoxyethylene fatty ethers (e.g., Brij™ S100). Clays, such as bentonite, may be particularly useful as a wall forming material for such uses. In other, certain embodiments, binders can be chosen specifically to prepare granules for aquatic uses, such as cleanup of oil spills and the like in marine settings. Suitable binders for such purposes can include hydrophobic materials such as waxes, paraffins, polycaprolactone, ethylene-vinyl acetate copolymers, polypropylene carbonate, poly(tetramethylene oxide) poly(ethylene adipate), poly (trans-butadiene), and thermoplastic polyurethane (e.g., Carbothane TPU). Again, various clays may be particularly useful as an absorbent, wall forming material in such uses. Biological agents and similar materials effective for decomposition of hydrocarbons or effective for otherwise modifying spilled liquids to improve ease of cleanup may be included with the hollow core granules. Such components provided as solid that may be includes as a wall forming material, may be liquids that are combined with solid, wall forming materials, may be liquids that are blended with the binder, or may be included in the hollow core granules in any other suitable manner.

In some embodiments, the ability to provide a given material in the presently described hollow core form can be beneficial in relation to processing and use of the material. For example, many solid materials that are typically sold in a particulate form can exhibit significant dusting during handling due the presence of fines (i.e., a quantity of the material that is significantly smaller in size than the average size of the remaining quantity of the material). Fines may be inherently present in a mass of certain materials due to the manufacturing process, due to unavoidable crushing of the particles during storage and/or handling, or due to other reasons. Reduced dusting can be achieved according to the present disclosure since the individual particles forming the granules of the hollow core structures are retained in the shell/wall of the individual granules due to the presence of the binder. Since fine particles are bound or adhered together in the wall of the granules and/or in one or more layers of the wall, such fine particles are less likely to become airborne during movement of the particles. As such, the provision of compositions in a hollow core structure where individual particles of a solid material are combined with a binder to form one or more walls or shells surrounding a hollow core, the amount of dust associated with a given mass of the material can be significantly reduced.

In addition to reducing dusting, the structure of the hollow core granules (i.e., having a wall of particles and binder surrounding a hollow core) can also lead to improvements in flowability and/or pourability of the material. Because the individual granules of the hollow core structures are formed through agglomeration around a binder particle, the individual granules can exhibit a substantial degree of uniformity in one or both of size and shape. This can lead to an improved appearance relative to particles of the same material in its typical, dense form that may have a significantly wide range of particle sizes and/or shapes. On the other hand, the present hollow core structures can be provided with a substantial uniformity of size so that the average particle size may vary, for example, by less than 20%, less than 15%, less than 10%, less than 5%, or less than 2% relative to the median particle size. Such uniformity can improve the manner in which the individual granules interact with one another during movement so that hollow core structures flow more easily along and around one another.

In some embodiments, hollow core granules according to the present disclosure can be configured to provide pH modification. As such, the hollow core granules can be configured for addition to a substantially acidic material or site (e.g., having a pH of less than 7, less than 6, less than 5, less than 4, or less than 3) in order to make the material or site less acidic, substantially neutral (e.g., in the range of about 6 to about 8 or about 6.5 to about 7.5), or basic (e.g., pH greater than 7, greater than 8, greater than 9, greater than 10, greater than 11, or greater than 12). Alternatively, the hollow core granules can be configured for addition to a substantially neutral material or site in order to make the material or site substantially acidic as defined above or substantially basic as defined above. Alternatively, the hollow core granules can be configured for addition to a substantially basic material or site as described above in order to make the material or site less basic, substantially neutral as defined above, or substantially acidic as defined above. Configuration for pH modification can be achieved by utilizing an acidic component as the wall forming material, using a basic component as the wall forming material, utilizing a buffer as wall forming material, or using some combination of acidic components, basic components and buffers as the wall forming material. Acidic components can include organic acids, such as oxalic acid, tartaric acid, citric acid, maleic acid, etc. that are typically available in solid form. Various salts likewise may be utilized in relation to the ability to release ions upon solubilization that can be effective at lowering pH of the surrounding environment. Basic components can include materials such as oxides of various metals, as well as various salts that release ions upon solubilization that can be effective to increase pH of the surrounding environment, such as various carbonates, hydroxides, and the like. Buffers, for example, can be prepared with mixtures of salts of similar materials that will release ions in solution at an appropriate level to maintain a substantially consistent pH in a local environment. Once provided as hollow core granules, the pH modifying hollow core granules can be added to liquids, for example, and achieve rapid solubilization to modify pH in a manner as noted above.

In light of the foregoing, it can be seen that the present disclosure can encompass a wide variety of products that can exhibit very useful properties, including improvements relative to typical forms of the same material(s) when not in a hollow core form as described herein. This can extend to a number of chemicals and compounds that are typically useful in various products in their salt form. Many salts are made or found in nature in a solid form at generally ambient conditions and, as such, a wide variety of salts may be utilized as a wall forming material in hollow core granules according to the present disclosure. Salts that can be provided in the form of hollow core granules according to the present disclosure can be organic or inorganic. In some embodiments, salts suitable for preparation as hollow core granules can include those with cationic groups such as aluminum, ammonium, bismuth, calcium, chromium, copper, germanium, iron, lithium, magnesium, manganese, nickel, palladium, platinum, potassium, silver, sodium, sulfur, tin, titanium, tungsten, vanadium, zinc, and zirconium. In further embodiments, salts suitable for preparation as hollow core granules can include those with anionic groups, such as acetates, aluminates, ammonium sulfates, benzoates, borides, bicarbonates, bromates, bromides, carbides, carbonates, chlorides, chromates, ferrites, fluorides, hydrides, hydroxides, iodates, iodides, lactates, manganates, nitrates, nitrides, oxalates, oxides, perchlorates, phosphates, phosphides, silicates, silicides, stearates, sulfates, sulfides, titanates, tungstates, vanadates, and zirconates. Non-limiting examples of specific salts that can be utilized in hollow core granules include calcium carbonate, sodium chloride, sodium carbonate, sodium bicarbonate, sodium percarbonate, sodium sulfate, sodium carbonate peroxide, potassium chloride, magnesium carbonate, magnesium sulfate, and the like.

The ability of compounds, such as salts, to exhibit surprisingly improved properties when in the form of a hollow core structure can be shown in relation to the example embodiment of sodium bicarbonate (NaHCO₃) or baking soda. Sodium bicarbonate is known to have a wide variety of uses, one example of which is use as a deodorizer in light of the ability of the material to absorb odor causing compounds, such as sulfurous compounds. As further described herein, particles of sodium bicarbonate can be combined with binders, such as PEG, paraffin, or other binders, to form granules wherein a hollow core is surrounded by one or more walls/shells comprising the sodium bicarbonate and the particular binder. The resulting hollow core sodium bicarbonate granules can provide improved odor-absorbing properties over known forms of sodium bicarbonate where the material is provided simply as a powder or in a solid mass of larger size. Hollow core sodium bicarbonate granules thus can be particularly useful as deodorizing agents for use in a variety of environments, including refrigerators, trash cans, garbage cans, pet litter boxes, and the like. This is shown in Example 12 below where sodium bicarbonate in the hollow core form was shown to exhibit improved malodor reduction in relation to materials such as ammonia and sulfur.

Hollow core sodium bicarbonate is thus an example embodiment of a substantially pure compound that can be upgraded for improved use through modification so that the substantially pure compound is combined with a binder to form hollow core granules. As such, a hollow core sodium bicarbonate granule would differ from the typical form of sodium bicarbonate in that the granules comprise particles of the sodium bicarbonate in a shell/wall with a binder so that the shell/wall surrounds a hollow core. The binder may be substantially inert in relation to the desired use of the sodium bicarbonate; however, in some embodiments, binders may be chosen to compliment the intended use and thus provide an additive effect to the sodium bicarbonate itself. The hollow core sodium bicarbonate would also differ from the typical form of sodium bicarbonate in relation to the improved properties as already discussed above—e.g., improved absorption and/or adsorption, improved solubilization, reduced weight, and other properties.

As seen from the example embodiment where sodium bicarbonate is used as the wall forming material for the hollow core structures, it is possible according to the present disclosure to structure the wall forming material into a higher order format so that usefulness and efficacy of the wall forming material can be improved as a standalone product. Such improvements, however, are not limited to sodium bicarbonate, and other wall forming materials as described herein can likewise benefit by the restructuring the material from its native format (i.e., the typical, fully dense form of the solid material) to the walled format where particles of the material are positioned in a wall with a binder surrounding a hollow core. Likewise, such improvements are not limited to uses as standalone products. Rather, individual materials, such as sodium bicarbonate, that have been upgraded into granules with the hollow core format can be used as components of various mixtures and compositions defining other types of products. For instance, using the example of hollow core sodium bicarbonate granules, such upgraded form of the material can be utilized as an ingredient is a number of useful products. Presently, sodium bicarbonate in its typical, fully dense form, finds use in other formulations, such as laundry detergents, dish detergents, carpet cleaners/deodorizers, animal litters, and personal care products, such as deodorants/antiperspirants, and dental care items (e.g., toothpastes). Any one or more of such products thus may be modified and improved by replacement of sodium bicarbonate in its typical form with hollow core sodium bicarbonate granules according to the present disclosure. The so-modified composition then can exhibit improvements arising at least from the improved functional aspects of the hollow core sodium bicarbonate granules. Of course, it is understood that sodium bicarbonate is utilized as an example embodiment, and the ability to provide improved products is not limited to the use of hollow core sodium bicarbonate granules, and such improvements may be achieved through upgrading of chemicals, compounds, and complex mixtures and compositions that may or may not include sodium bicarbonate as a component thereof.

Because a wide variety of materials may be used as the wall forming material, the present hollow core granules can be configured as a wide variety of products. Non-limiting examples of products that may include, in part or in whole, the hollow core granules of the present disclosure include cleaning compositions (e.g., laundry detergents, dish detergents, fabric cleaners, fabric deodorizers, abrasive cleaners, teeth cleaning compositions, disinfectants, etc.), cleaning composition additives (e.g., stain removers, whiteners, brighteners, bleaches, scent boosters, etc.), absorbents, adsorbents, deodorizers, odor control products, odor masking products, fertilizers, pesticides, animal litters, animal litter additives, and other consumer products and/or industrial products. Any of the foregoing may be a functional material as referenced above and may also be referenced as additives in that they can be added to other products to impart the desired function and/or may be provided as a stand-alone product that can be combined as needed with other products to achieve an additive result.

In one or more embodiments, products suitable for provision as hollow core granules can include one or more chemicals, compounds, or mixtures of materials that are effective as detergents/cleaners and/or as additives useful for combination with detergents/cleaners. Many cleaning products are provided in solid form, typically as powders or other particulate forms. Common examples of such compositions include fabric care items (e.g., laundry detergents for use in washing machines, upholstery cleaners, brighteners, whiteners, stain removers, scent boosters, and the like) and dishwashing detergents. According to the present disclosure, pre-existing cleaning compositions may be re-engineered into an upgraded format wherein one or more individual components of the mixture may be present in a hollow-core form. For example, sodium bicarbonate in such formulations may be replaced with hollow core granules of sodium bicarbonate. Other discrete components of the cleaning composition may alternatively or additionally be replaced with a hollow core version of the component. In other embodiments, the total powdered product may be modified so that the overall composition is in the form of hollow core granules. Powdered cleaning compositions may be a mixture of components that are blended into a substantially uniform powder or other particulate form. Rather than being utilized in the powdered form, the total mixture may be utilized as the wall forming material and mixed with a suitable binder so that the individual particles of the total cleaning composition are agglomerated with the binder in one or more wall of the formed granules with the hollow core format. Alternatively, the granules with the hollow core format may be prepared to have two or more walls/shells. In such embodiments, a first portion of a cleaning composition may be present as a first, inner shell or wall, and a second (or more) portion of the cleaning composition may be present in a second (or more) wall or shell external to the inner shell. More particularly, one or more components of a cleaning composition may be present as a first, inner shell or wall, and a second (or more) component(s) of the cleaning composition may be present in a second (or more) wall or shell external to the inner shell. In this manner, a timed release of individual components of the cleaner may be provided. For example, one or more outer shells in a dishwashing composition may provide a detergency function, and one or more inner shells of the composition may provide an enzymatic or different function that is more desirable in a later portion of the dishwashing cycle. In this manner, a single composition can be provided with timed release of different components of the composition. Similar effects can be achieved through layering in other compositions, such as laundry cleaning compositions. In addition to providing timed release, the provision of the compositions in the hollow core format can provide further benefits. For instance, re-engineering of powdered laundry detergents and similar formulations may be desirable, for example, to reduce overall product weight, improve solubility (and thus reduce the likelihood of detergent residue on cleaned articles), and the like.

A cleaning composition according to the present disclosure may comprise substantially only hollow core granules according to the present disclosure. The hollow core granules may include one or more chemicals, compounds, or the like having one or more cleaning application(s) as the wall forming material, and the wall forming material optionally may also include one or more carriers, fillers, inert materials, or the like that do not necessarily provide a cleaning function. The cleaning composition comprising substantially only hollow core granules may thus be configured as a substantially complete formulation for a designed use (e.g., a laundry detergent, a dishwashing detergent, etc.), or the cleaning composition comprising substantially only hollow core granules may be configured as an additive (e.g., a bleach, brightener, whitener, stain remover, deodorizer, etc.) that can be added to another composition for a desired end use. A cleaning composition according to the present disclosure may comprise hollow core granules in combination with non-hollow core components. For example, a cleaning composition may be provided as a mixture of components, and one or more of the components may be provided in the hollow core form while one or more of the remaining components may be provided in a non-hollow core form.

As is evident from the foregoing, a cleaning composition according to the present disclosure may be combination of materials defining the total cleaning composition or may be a more specialized product that is provided as an additive for cleaning products. Non-limiting examples thereof include additives, such as brighteners, non-bleach whiteners (including oxidizing materials), scent-boosters, enzymes, deodorizers, stain removers, and other materials that are useful in cleaning products. Moreover, as already noted previously, the ability to provide the compositions in the hollow core format can extend to liquid or semi-solid components as well. In particular, one or more liquid or semi-solid components may be absorbed, adsorbed, or embedded in or on a solid component of the cleaning composition or on an inert carrier that may harmlessly dissolve in the washing liquor and be removed.

In some embodiments, a cleaning product or composition according to the present disclosure may be a fabric cleaner or fabric cleaning composition. A fabric cleaner can be any product that is configured at least for use with textiles or fabrics, such as clothing, upholstery, carpets, rugs, bedding (e.g., sheets, blankets, duvets, bedspreads, quilts, mattresses, etc.), tapestries, and the like.

A fabric cleaner specifically may be a laundry detergent. Such compositions are known to include a number of components, including polymers, surfactants, builders, deodorizers, enzymes, oxidizers, bleaching components, salts, fragrance, and the like. Salts such as sodium sulfate, sodium carbonate, sodium bicarbonate, sodium chloride, potassium chloride, and the like particularly may be included in laundry detergents. Example embodiments of suitable polymers include polyethylene glycol (PEG) polymers of various molecular weights. Example embodiments of suitable surfactants can include anionics, nonionics, zwitterionics, ampholytics, cationics, and combinations thereof. One example of a laundry detergent includes C12-15 ethoxylated alcohols, sodium laureth sulfate, sodium sulfate, sodium carbonate, sodium bicarbonate, disodium distyrylbiphenyl disulfonate, modified acrylic copolymer, protease enzyme/amylase enzyme, sodium carbonate peroxide, potassium chloride, and fragrance. Such composition may be provided in a solid (e.g., powdered) format, and the solid detergent particles can be used as the wall forming material to provide the laundry detergent as hollow core granules. In some embodiments, a product according to the present disclosure can be a laundry detergent prepared by a method as described herein such that the laundry detergent comprises a mixture of hollow core granules and one or more further components that are effective in a laundry detergent composition. In further embodiments, a product according to the present disclosure can be a laundry detergent prepared by a method as described herein such that the laundry detergent comprises hollow core granules prepared such that a plurality of individual particles of at least one wall forming material comprise particles of a laundry detergent composition.

A fabric cleaner may also be provided in a more specialized form to provide a designed effect. Various functional formulations are possible to design products that can be used as additives in fabric cleaning, and particularly in laundry care. Example embodiments of such additive formulations include scent boosters, stain removers, brighteners, whiteners, bleaches, and the like. One example of a scent booster includes a sodium chloride builder, fragrance, a sodium bicarbonate builder, a hydrated silica process aid, a sorbitan oleate surfactant, and colorant. Such composition may be provided in a solid (e.g., powdered) format, and the solid particles can be used as the wall forming material to provide the scent booster formulation as hollow core granules. One example of a stain remover includes a sodium carbonate, sodium percarbonate, C12-15 linear alcohol ethoxylate, fragrance, and blue salt. Such composition may be provided in a solid (e.g., powdered) format, and the solid particles can be used as the wall forming material to provide the stain remover formulation as hollow core granules. Other additive formulations for fabric care may likewise be formulated for providing the product as hollow core granules.

Dishwashing detergents likewise can be formulated wherein powdered compositions may be provided in the form of hollow core particles. Any known, solid dishwashing detergent may be so formulated. Further, individual components of a dishwashing detergent may be individually formulated as hollow core granules that may be provided as additive or that may be otherwise admixed with other components of a dishwashing detergent that are not in a hollow core format.

Other types of household cleaners may also be subject to such re-engineering. For example, in the field of fabric care, carpet cleaners or other upholstery cleaners are often provided in a powdered form, and such compositions can be improved by re-engineering into a hollow core format as described herein. For example, sodium bicarbonate may be used in carpet cleaners to remove odors as well as provide a cleaning effect, and the provision of sodium bicarbonate as the wall forming material of hollow core granules can be effective to improve activity in the end use because of the improved absorption and/or adsorption provided through such format. Other components of such cleaners may additionally or alternatively be included in the product in the hollow core format. Likewise, an entire carpet or upholstery cleaning composition may be provided as hollow core granules that can be applied to the material to be cleaned. The applied granules may be vacuumed or otherwise removed at the appropriate time or, in some embodiments, the hollow core granules may be acted upon through outside force (such as foot traffic or use of machinery) to effect breakdown of the hollow core granules into a finer, powered form. Such mechanical action can be effective to improve the cleaning effect, improve odor removal, or the like prior to removal of the composition, such as through vacuuming of carpet or the like.

In some embodiments, the hollow core granules may be particularly configured for degradation upon application of an external force. The external force may be a rubbing, wiping, scrubbing, or other physical pressure typically applied during cleaning of a surface. More specifically, during the application of the external force, the hollow core granules can be configured to break into a plurality of parts comprising individual groups of the particles of the wall forming material. In other words, the overall granule will break into a plurality of subunits having a size that is less than the size of the original granule but is greater than the size of individual particles of the wall forming material since a plurality of the particles will still be agglomerated in each of the plurality of subunits. It is possible, however, that during application of the force, individual particles may be freed along with the plurality of subunits. During further application or continued application of the force, the plurality of subunits can further degrade into even smaller subunits and/or into the individual particles of the wall forming material.

Similar to cleaning products as discussed above, usefulness of hollow core structures can particularly extend to abrasive-type cleaners. An abrasive-type cleaner as used herein is intended to mean a cleaner wherein cleaning is achieved at least in part through mechanical action of the solid particles physically removing deposits from a surface through a scrubbing action. Such cleaners may also achieve cleaning through detergency in addition to the mechanical scrubbing of the particles along the surface to be cleaned. As discussed herein, the granules of the hollow core structures have at least one wall formed of smaller particles of the wall forming material. When the wall forming material is effective as an abrasive-type cleaner, the hollow core granules formed therefrom can exist as relatively large particles that can provide a “rough” abrasive surface, and the mechanical scrubbing action can cause the hollow core granules to gradually degrade into finer particles. The result is similar to sanding of a surface wherein a low grit, rough surface is initially used for bulk removal of material from the surface, and higher grit surfaces are used thereafter for smoothing. The granules of the hollow core structure can similarly function as a low grit, rough abrasive for bulk removal of residues and buildup and, as the granules degrade into the finer, wall forming particles, such particles function as a higher grit abrasive to provide a finer, cleaning effect for removal of smaller traces of the residues and buildup. Further, the binder material can be chosen to control how easily the hollow core granules fracture, to control how quickly the hollow core granules will dissolve in a solvent, and also to provide additive cleansing effects. In addition, the hollow core format can impart haptic feedback to the user as efficacy of the abrasive cleaning. The larger hollow core granules will impart vibrations that are noticeably different from the haptic feel of the finer, wall forming particles. Likewise, since the hollow core granules can be configured to fracture under stress, such as application of pressure when cleaning, the breaking of the granules into the finer, wall forming particles will also provide haptic sensations of how the cleaning action is progressing. Thus, the hollow core granules can be configured to break down into successfully smaller sized particles to provide a layered scouring efficacy due to differences in cleaning ability provided by the different sizes of the wall forming particles, the intact hollow core granules, and the intermediate sized sections of the granule wall as it breaks apart.

Similar to abrasive cleaners as noted above, hollow core granules of the present disclosure likewise may be used as polishing agents. In particular, one or both of the wall forming material and the binder can be chosen to provide polishing attributes. Likewise, the particles of the wall forming material may be chosen in size to provide a desired level of abrasiveness needed to achieve the polishing effect without unduly scratching or marring the material being polished. Otherwise, hollow core polishing granules may be functionally similar to the abrasive cleaning granules described above.

In some embodiments, the presently disclosed hollow core granules can be utilized in personal care items. A particular example is in the area of deodorants/antiperspirants. A further example can be exfoliating products where the hollow core granules can provide a relatively rough level of exfoliation in the initial, larger granule size and provide continually smoother levels of exfoliation as the hollow core granules break down into the individual wall forming particles that are significantly smaller in size. The binder material in such applications can be customized to provide additional skin cleansing effects and/or to provide a lubricating effect to the skin as the granules break apart and/or as the granules are solubilized in water.

Dental care products are further examples of products that can exhibit improvements through utilization of hollow core granules. More particularly, hollow core granules as described herein may be utilized in forming toothpaste compositions. One or more of the individual components of the toothpaste composition may be provided as hollow core granules that are incorporated into the overall paste, gel, or similar composition use for teeth cleaning. For example, sodium bicarbonate is a common ingredient in toothpaste compositions, and the sodium bicarbonate may be present in the composition as hollow core granules. Likewise, since many teeth cleaning compositions utilize at least mildly abrasive particles, such particles may be incorporated into hollow core granules as at least one of the wall forming materials. Further, the binder material may also be chosen to improve the activity of the wall forming material and/or to improve solubilization of the wall forming material for rapid deployment during brushing. Alternatively, the overall teeth cleaning composition may be re-engineered as a hollow core structure that then can be combined with a substrate or carrier material to from a paste, gel, or the like.

The use of hollow core granules can also provide for new teeth cleaning formulations. For example, rather than incorporating hollow core granules into a teeth cleaning gel or paste, the hollow core granules may comprise substantially the entirety of the teeth cleaning composition. In an example embodiment, a complete or substantially complete teeth cleaning composition can be utilized as the wall forming material so that the formed, hollow core granules are effective as “toothpaste bits” that can be poured into the mouth for teeth cleaning. Similarly, a plurality of hollow core teeth cleaning granules may be combined into a tablet form or similar so that a single “tablet” may be inserted into the mouth for teeth cleaning. More particularly, the toothpaste bits or toothpaste tablet, once inserted into the mouth, may be chewed so that the abrasive particles remove debris and other materials from the teeth and/or gums of the user. Again, the choice of binder material may be effective to cause the teeth cleaning granules to break apart easier or to persist longer so that effective use time may be customized. Further, the binder may be effective in providing teeth cleaning properties so that the binder is at least partially effective for removing debris or other materials from the teeth and/or gums. As with other abrasive cleaning hollow core granules, the teeth cleaning hollow core granules can provide varying levels of cleaning efficacy as the hollow core structures break apart into successfully finer sized particles.

Cleaners, detergents, and similar products may be prepared as substantially “simple” products with only a few ingredients, and one or more of the relatively few components used in such components may be present in a hollow core format, or substantially the entire composition defining the product may be present in the hollow core format. Others of such types of products may be relatively complex in relation to include a larger number of components. Again, any one or more of the components may be in the hollow core form or substantially the entire composition may be in the hollow core form. In some types of composition, however, it may be more typical for only primary components thereof to be in the hollow core form. As such, only primary components may be discussed herein in relation to being in a hollow core form. It is understood, however, that many consumer products may include a wide variety of classes of materials, and any further components that may be utilized in any product or article of manufacture encompassed by the present disclosure, including animal litters, laundry products, dishwashing products, personal care items, and the like, may be included in such products or articles of manufacture in the hollow core form as described herein. It is thus expressly intended that any of the following additives may be used in any product or article of manufacture wherein which components are typically understood to be used: fillers, binders, preservatives, fragrances, salts (e.g., carbonates, bicarbonates, chlorides, etc.), optical agents (e.g., brighteners and/or whiteners), disinfectants, enzymes, antimicrobials, oxidizers, deodorants, pH adjusters, dyes, coloring agents, and the like.

In some embodiments, hollow core granules as described herein can be useful in forming nutritional supplements for oral ingestion. This can provide for a wide variety of forms of the nutritional supplements to provide for improved properties whether the articles are configured to be chewable or are configured for swallowing whole. In relation to the latter format, many nutritional supplements suffer from poor release of the vitamin(s), mineral(s), fiber, probiotics, enzymes, amino acids, proteins, or other supplemental agent(s) typically found in various nutritional supplements. This often arises from poor solubility of the overall pill or tablet form. As discussed above, however, hollow core structures according to the present disclosure can exhibit improved solubility because of the ability of the wall to rapidly break apart into the significantly smaller particles used as the wall forming material and because of the ability to customize the binder to the environment where dissolution will be carried out so that the binder itself will readily solubilize in a contacting solvent. Because of the much higher surface area afforded by the small, wall forming particles, the rapid break down of the larger hollow core granules into the individual wall forming particles can provide for rapid release and quick uptake of the nutritional supplement(s) in the digestive system of a user. Moreover, the hollow core format can enable combination of various components for timed release. As discussed otherwise herein, coatings, encapsulation, and other methods can be employed to provide a quantity of the individual particles of one or more of the wall forming materials in a delayed release or sustained release form. Thus, when the nutritional supplement hollow core granules are ingested, at least a portion of the nutritional supplement used as the wall forming particles may provide for substantially immediate release (if desired), and at least a portion of the nutritional supplement used as the wall forming particles may provide for delayed and/or sustained release (if desired). Similarly, as not all nutritional supplements are readily absorbed in the stomach and/or may be partially or completely degraded in the stomach, the present disclosure allows for providing at least part of the nutritional supplement particles in a coated or encapsulated form that will survive the high acid environment of the stomach but be released in the small intestine for necessary absorption. Thus, the ability to provide different nutritional components in different formats allows for highly customizable nutritional supplement compositions with the nutritional materials present as the wall forming materials of the hollow core granules.

Similar to nutritional supplements, the hollow core granules can be configured as other personal care products that are configured for oral ingestion. For example, laxatives, antacids, and similar materials may be used in the hollow core granules. Materials such as PEG are known to be functional as laxatives, and hollow core granules may be prepared using a PEG binder for wall forming materials that may be substantially inert, that may also be configured as a laxative or stool softener, or may provide additional benefits, such as being a fiber supplement, being an antacid (e.g., sodium bicarbonate), or the like.

Since some users may have difficulty with swallowing pills, tablets, capsules, or the like, the present hollow core granules can be configured so that the nutritional supplement is in a chewable format. Specifically, the nutritional materials may again be used as the wall forming material of the hollow core granules, but the granules can be configured for ease of chewing and/or rapid dissolution in the mouth of a user so that the supplements can be provided in a convenient form (e.g., solid dosages versus liquid dosages) while still being easily ingested. Moreover, a variety of additives can be combined with the nutritional supplements to provide the hollow core granules in a palatable configuration. For example, sweeteners, flavorants, or other ingestible materials may be used as a portion of the wall forming material so that nutritional components that may otherwise be bitter, sour, or the like may be masked by the additives. Moreover, at least a portion of the binder likewise may be configured to impart palatable qualities to mask any unpleasant tastes associated with the nutritional supplements themselves. The nutritional supplement including vitamin(s), mineral(s), fiber, probiotics, enzymes, amino acids, proteins, or other supplemental agent(s) typically found in various nutritional supplements, can be provided in a bulk format wherein a mass or volume of the hollow core granules are provided with dosing instructions for an amount of the hollow core granules to ingest to deliver the daily recommended dosage or other dosage of the supplement(s) included therein. Alternative, a pre-dosed amount of the hollow core granules can be combined into a single unit, such as through the use of binding agents so that the hollow core granules are retained together as blocks, wafers, or similar unitary formats that a user may chew to release the hollow core granules therefrom.

An example embodiment of a nutritional supplement is a vitamin D supplement, which comprises dextrates, microcrystalline cellulose, magnesium stearate, chamomile powdered extract, flavor, and vitamin D. The components can be formulated and then used as the wall forming material in hollow core granules as described herein. Any nutritional supplement may be similarly formulated for preparation of the supplement in the hollow core format.

In some embodiments, the present, hollow core structures can be particularly useful in forming animal litters. As previously mentioned herein, clays are often a primary component of animal litter products due to the relatively low cost thereof and the particularly good efficacy for liquid absorption. Clays, however, are relatively dense and cause animal litter products to be quite heavy with commercially sold quantities requiring as much as 30 to 40 pounds of a clay-based litter to fill a large-size litter tray. Clays, however, can be particularly amenable for use as a wall forming material to produce the clay as a hollow core granule having a wall comprising smaller particles of the clay and a binder. The resulting, hollow core clay granules can thus be particularly useful in forming an animal litter having a significantly lower weight and even improved absorption properties. This can extend to animal litters having hollow core clay granules as well as hollow core granules formed from different wall forming materials.

The present disclosure thus can provide animal litter compositions that include at least one component thereof in the form of hollow core granules and that can exhibit improved properties including, but not limited to, reduced overall composition weight. Hollow core granules can be present in the animal litter in a defined amount, such as an amount of about 1% or greater by weight based on the total weight of the animal litter composition. In further embodiments, one or more types of hollow core granules may be present in the animal litter composition in amounts (independent of one another) of about 1% to about 95% by weight, about 2% to about 75% by weight, about 3% to about 60%, or about 5% to about 50% by weight based on the total weight of the composition. In some embodiments, a material present as hollow core granules may be present in a relatively low concentration, such as about 1% to about 10%, about 1.25% to about 7.5%, or about 1.5% to about 5% by weight based on the total weight of the animal litter composition. This may be the case, for example, in relation to ingredients such as sodium bicarbonate, which may be useful as a deodorizing component, fragrances, or other components typically present in animal litters. In further embodiments, a material present as hollow core granules may be present in a relatively high concentration, such as about 10% to about 90%, about 20% to about 85%, or about 25% to about 75% by weight based on the total weight of the animal litter composition. This may be the case, for example, in relation to ingredients such as liquid absorbents (e.g., clay), fillers, or the like. In other embodiments, the hollow core granules in an animal litter may be defined in relation to the volume ratio of the material since the hollow core version is expected to be significantly lighter than a non-hollow core version of the same material. For example, the total content of hollow core granules in an animal litter may be in the range of about 5% to about 98%, about 10% to about 95%, about 20% to about 90%, or about 30% to about 80% by volume based on the total volume of the animal litter composition. Other concentration ranges as already described above may be utilized on a volume basis. This can include low concentration components and/or high concentration components.

Animal litters may include a variety of components, and it is understood that animal litter compositions according to the present disclosure may include one of the following components in the form of hollow core granules. Likewise, the present animal litter compositions may include two, three, four, or even more of the following components in any combination in the form of hollow core granules. Non-limiting examples of the types of components that may be used in animal litters and that may be present in the form of hollow core granules include liquid absorbents, fillers, clumping agents (or clump enhancing materials), binders, preservatives, such as biocides (e.g., benzisothiazolinone, methylisothiazolone), de-dusting agents, fragrance, bicarbonates, and combinations thereof.

Fillers suitable for use in the present animal litter compositions can include a variety of materials that can be a non-absorbent, non-soluble substrate, or can be an absorbent substrate. In one or more embodiments, useful fillers can include absorbent substrates, such as non-clumping clays. Non-limiting examples of useful non-clumping clays include attapulgite, Fuller's earth, calcium bentonite, palygorskite, sepiolite, kaolinite, illite, halloysite, hormite, vermiculite or mixtures thereof. Suitable fillers according to the present disclosure also can include a variety of non-absorbent, non-soluble substrates, such as non-clay substances. Non-limiting examples of non-clay materials that can be used include zeolites, crushed stone (e.g., dolomite and limestone), gypsum, sand, calcite, recycled waste materials, and silica. As examples, the animal litter composition can comprise about 0% by weight to about 75% by weight, about 10% by weight to about 70% by weight, about 25% by weight to about 65% by weight, or about 40% by weight to about 60% by weight of one or more fillers based on the total weight of the animal litter composition or by volume based on the total volume of the animal litter composition. Such fillers may be present in a typical, non-hollow core format or may be present as hollow core granules or may be present as one of a plurality of components used as the wall forming material for hollow core granules.

Description of suitable clumping agents is provided in U.S. Pat. No. 8,720,375 to Miller et al., the disclosure of which is incorporated herein by reference. Useful clumping agents are those materials suitable to promote adhesion of the fine size particles of litter granules to each other as well as adhesion of the particles to form agglomerates when wetted. Preferably, the clumping agent allows the formation of a gelled agglomerate when exposed to a liquid, such as animal urine. A clumping agent may be provided in admixture (e.g., in particle form) with the further components of the animal litter. In some embodiments, the clumping agent can be provided as a coating on at least a portion of the other components forming the animal litter (e.g., as a coating on at least a portion of the filler material). Such coatings may be provided by any known method, such as spraying. If desired, a clumping agent may be provided as an outer layer/wall on a hollow core structure as already described above. For example, a clumping agent may be coated on a hollow core structure having a clay wall and/or a sodium bicarbonate wall. Non-limiting examples of materials suitable for use as a clumping agent include naturally occurring polymers, semisynthetic polymers, and sealants. Example embodiments of naturally occurring clumping agents include various starches, including corn starch, various gums such as gum arabic, gum karaya., gum tragacanth, gum Bhatti, guar gum, and xanthan gum, as well as alginates, carrageenan, pectins, dextran, gelatin, gluten, dried plants of the Plantago family, vinyl polymers, including polyvinyl alcohol, polyvinyl esters such as polyvinyl acetate, polyvinylpyrrolidone, polyvinyloxazolidone, polyvinylmethyloxazolidone, copolymers and mixtures thereof. Example embodiments of semisynthetic polymers include cellulose ethers (e.g., methylcellulose, hydroxyethyl cellulose, hydroxypropyl cellulose, ethylhydroxyethyl cellulose, methylhydroxypropyl cellulose, carboxymethylcellulose, hydroxypropylmethylcellulose or mixtures thereof), and guar gum derivatives. The amount of any clumping agent that is present in the animal litter composition can vary based upon the total composition. For example, it can be useful to include a greater amount of clumping agents when a greater amount of non-absorbent fillers is used. In some embodiments, clumping agents can be present in a total amount of 0.1% by weight to about 6% by weight, about 0.2% by weight to about 5.5% by weight, about 0.3% by weight to about 5% by weight, or about 0.5% by weight to about 4% by weight, based on the total weight of the litter composition or by volume based on the total volume of the litter composition.

To the extent that one or more binders, preservatives, de-dusting agents, fragrances, bicarbonates, or the like it included, such materials may be present independently in any amount up to about 5% by weight, up to about 2% by weight, up to about 1% by weight, or up to about 0.5% by weight, such as about 0.01% by weight to about 5% by weight, to about 4% by weight, to about 3% by weight, to about 2% by weight, or to about 1% by weight based on the total weight of the animal litter composition. Such amounts alternatively may be by volume based on the total volume of the litter composition. Further, it is understood that any one or more of such materials may be expressly excluded from the present animal litter composition.

In some embodiments, a product according to the present disclosure can be an animal litter prepared by a method as described herein such that the animal litter comprises a mixture of hollow core granules and one or more further components that are effective in an animal litter composition. In further embodiments, a product according to the present disclosure can be an animal litter prepared by a method as described herein such that the animal litter comprises hollow core granules prepared such that a plurality of individual particles of at least one wall forming material comprise particles of a clay or particles of sodium bicarbonate.

In addition to litter compositions, the present disclosure further can extend to a variety of additives that may be used with cat litters. For example, various odor-masking agents or deodorizers may be available in solid form, and such materials may be provided in the form of hollow core granules as described herein. Non-limiting examples of litter additives that may be provided in the form of hollow core granules include deodorizers, clumping agents, de-dusting agents, fragrance, odor masking agents, and the like. Any one or more materials useful in pet litters may be formulated individually or together in the form of hollow core granules to provide litter additive products.

As already discussed herein, the hollow core granules can be particularly useful in fertilizer compositions. Fertilizers are typically recognized as compositions that provide nitrogen, phosphorus, potassium, or other minerals needed for plant health such as so-called micronutrients (e.g., boron, chlorine, copper, iron, manganese, molybdenum, and zinc). The fertilizer particularly can be a nitrogen source, and examples of nitrogen sources can include materials wherein nitrogen can be provided as one or more of: amides, such as urea, urea ammonium nitrate (UAM), or polymer encapsulated urea; ammonium compounds, such as ammonium bicarbonate, ammonium sulfate, ammonium chloride, and the like; and nitrates, such as sodium nitrate, calcium nitrate, and ammonium nitrate. The fertilizer particularly can be a phosphorus source, and examples of phosphorus sources can include materials wherein phosphorus may be provided in its elemental form or in phosphate salts. The fertilizer particularly can be a potassium source, and examples of potassium sources can include materials wherein potassium can be provided in an elemental form or in a salt form, such as potash. The fertilizer particularly can be micronutrient source, and examples of micronutrient sources can include materials wherein micronutrients as noted above or otherwise accepted as such are provided. Other materials that may be considered to fall within the group of fertilizers as used herein can include materials that are commonly used to modify soil pH. Materials for increasing soil pH can include lime, which can more particularly be present as calcium carbonate, or calcitic limestone, or as dolomitic limestone, which is a combination of calcium carbonate and magnesium carbonate. Materials for reducing soil pH include sulfur or sulfur containing minerals or compounds.

Any one or more materials suitable for use as a fertilizer may be used as a wall forming material for hollow core granules as described herein. Fertilizer materials available in a solid form may thus be provided as relatively small particles that define one or more walls/shells in a hollow core granule. Upon delivery to a site of use, the fertilizer particles may be released from the relatively larger granules for solubilization in the soil. Fertilizer materials available in a liquid form may be combined with particles of an absorbent or adsorbent material, such as clay, so that the liquid fertilizers are entrained thereby. The particles of the combined materials may then be used to prepare hollow core granules of the fertilizer. Upon delivery to a site of use, the fertilizer may be released from the carrier particles, and the remaining clay particles may be incorporated into the soil or other site of delivery without harm.

Various chemicals and compounds useful as fertilizers can exhibit different dissolution rate or release rates. In particular, there are variously know “fast release” fertilizers and “slow release” fertilizers. Where it is desirable to provide combinations of fertilizers with different release rates, particles of a fast release fertilizer and particles of a slow release fertilizer can be combined in desired ratios and used as the wall forming component for preparing granules of hollow core structures as described above. Thus, the resulting fertilizer granules will have a wall surrounding a hollow core wherein the wall comprises the fast release fertilizer particles and the slow release fertilizer particles in the designed ratio. Upon application to a site in need of fertilization, the fast release fertilizer particles will provide immediate fertilization, and the slow release fertilizer particles will remain for the expected time for slow release thereof. The same principle can apply to any number of solid materials with differing dissolution and/or release rates so that many types of controlled release granules can be prepared. Alternatively, fertilizers with different release rates may be provided in separate hollow core granules. The separate hollow core granules can then be combined at the time of use in a desired ratio.

In some embodiments, controlled release of fertilizers may be particularly achieved through use of encapsulation methods as already described above. For example, certain fertilizers may be provided in a microencapsulated or other encapsulated form so that the fertilizer material is only released after dissolution, degradation, of the like of the encapsulating material. The encapsulated fertilizer particles may be used as the wall forming material (alone or in combination with other fertilizer particles in a rapid release and/or controlled release form) to provide hollow core fertilizer granules that will exhibit controlled release. Coating technology may also be utilized as a means for provision of liquid components as the wall forming material. As an example, particles of polymer encapsulated urea may be used as a wall forming material that can be combined with a binder to prepare hollow core fertilizer granules. In further embodiments, controlled release may be achieved through choice of binder material. For example, binders may be chosen based on a specific solubility so that fertilizer particles in the wall of the hollow core granules will only be released upon solubilization of the binder. Likewise, hollow core fertilizer granules may be provided with multiple walls, shells, coatings, etc. with different properties to control release of one or more fertilizer components therefrom. The different walls, shells, coatings, etc. may release their fertilizer components only under specific conditions and/or may exhibit different solubilization properties so that a controlled release may be achieved based upon the solubilization/degradation of the multiple walls, shells, coatings, etc. This can be characterized as a multi-stage release fertilizer or controlled release fertilizer.

Fertilizer compositions may be applied to a site of use by a variety of methods. Solid fertilizer compositions may be applied directly to the soil for release of the fertilizer component(s) through solubilization over time. Other fertilizer compositions, however, may be preferably admixed with a solvent and then sprayed onto the delivery site. The improved dissolution rates provided by utilizing a hollow core format as described herein can be beneficial for such uses. In particular, the solid, hollow core fertilizer granules may be provided in bulk and then the desired mass or volume of the solid, hollow core fertilizer granules can be added to a tank sprayer or similar apparatus immediately prior to application. Because the hollow core format can significantly lessen the time to dissolution of the material, the hollow core fertilizer granules can rapidly dissolve in the tank sprayer or similar apparatus for application at the site of use without significant delay. The hollow core granules can thus be particularly useful in large scale farming or the like. Likewise, since the hollow core format significantly reduces the weight of the material, this can further improve ease of application of the fertilizer.

Rapid dissolution of the hollow core granules can also be advantageous for environmental safety. Fertilizer run-off can be problematic due to passage into waterways (e.g., rivers, streams, ponds, or even wastewater drainage. Fertilizer particles can be easily washed into such waterways by heavy rains if the particles have not substantially degraded or dissolved. Since the hollow core format can significantly hasten dissolution and/or degradation, the use of the hollow core format for fertilizers can reduce such potential problems. As seen in the appended examples, hollow core fertilizer granules formed using bentonite as a wall forming material and PEG 8000 as a binder have been shown to dissolve in water in a matter of second. This illustrates the ability according to the present disclosure to provide hollow core fertilizer granules in a form that are conveniently stored and transported (solid versus liquid), are easier to handle (e.g., having a reduced weight), and will dissolve rapidly once contacted with water to release the fertilizer component.

In example embodiments, hollow core fertilizer granules can be prepared with a variety of wall forming materials and a variety of binders. For instance, various clays (e.g., bentonite), salts (e.g., sodium bicarbonate, magnesium sulfate, etc.), and similar materials can be useful as a wall forming material along with any specific, solid fertilizer materials that may be included in the granules. Clays are particularly useful since such components can effectively bind various types of fertilizers, release the fertilizers in the soil, and then remain in the soil as an inert additive. Polymer encapsulated fertilizers can particularly be utilized, encapsulated ureas and encapsulated phosphates being example embodiments. Suitable binders can include hydrophilic materials such as PEGs of various molecular weights (e.g., PEG 8000, PEG 12000, and/or PEG 35000), or similar materials that will readily solubilize in soil for degradation of the granules and release of the fertilizer materials therefrom.

Other types of materials that may be desired for application in the environment may likewise benefit from provision in a hollow core format. Pesticides, for example, can include a wide variety of chemicals, compounds, and the like that are configured for control of pests, insects, and the like. The group of pesticides can include, for example, algaecides, antimicrobials, biopesticides, disinfectants, fungicides, herbicides, insecticides, miticides, molluscicides, ovicides, repellants, rodenticides, and the like. Use of the term “pesticide” herein may thus be understood as referencing any of the above, example embodiments of materials useful for control of undesired species (pests, insects, weeds, etc.). Many such materials are commonly sold in particle form for application with spreaders or for hand application. Pesticides may be designed so that improved performance is achieved by wetting following application. This can be necessary to ensure that the active ingredients are dispersed in the soil or other point of application and/or to limit contact of the active ingredients with humans, pets, or wild animals. Moreover, wetting for dispersion and soil contact can be necessary to prevent unnecessary run-off of the potentially dangerous chemicals into waterways.

Any chemical, compound, or similar material effective as a pesticide may be utilized in forming a hollow core granule according to the present disclosure. The pesticide specifically may be considered to be an active agent in light of previous recognition thereof as exhibiting a pesticide activity. Non-limiting examples of pesticides that may be used according to the present disclosure and that may be considered to be a pesticide active agent include bifenthrin, acephate, carbaryl, cyfluthrin, 2,4-dichlorophenoxyacetic acid, trifluralin, chlorpyrifos, allethrins, cypermethrin, disulfoton, 2,6-dichlorobenzonitrile, metolachlor, cyhalothrin, hydramethylnon, atrazine, chlorothalonil, myclobutanil, dicamba, azadirachtin, captan, diazinon, carbofuran, methomyl, deltamethrin, propiconazole, borate, dinotefuran, dithiopyr, isoxaben, prodiamine, quinclorac, sethoxydim, iron(III) phosphate, mancozeb, thiophanate-methyl, esfenvalerate, tebuconazole, resmethrin, glyphosate, malathion, permethrin, imidacloprid, fipronil, abamectin, spinosad, triclopyr, piperonyl butoxide, pendimethalin, oryzalin, and oxadiazon.

Any one or more pesticides may be utilized in a hollow core granule in a manner similar as already discussed above in relation to fertilizers. In particular, one or more pesticides may be used as a wall forming material for hollow core granules as described herein. Pesticides available in a solid form may thus be provided as relatively small particles that define one or more walls/shells in a hollow core granule via combination with one or more binders. Upon delivery to a site of use, the pesticide particles may be released from the relatively larger granules for solubilization and/or dispersion in the soil. Pesticides available in a liquid form may be combined with particles of an absorbent or adsorbent material, such as clay, so that the liquid pesticides are entrained thereby. The particles of the combined materials may then be used to prepare hollow core granules of the pesticides. Upon delivery to a site of use, the pesticide may be released from the carrier particles, and the remaining clay particles may be incorporated into the soil or other site of delivery without harm.

Pesticidal hollow core granules may be configured with a single pesticide or with a combination of pesticides having the same or different activities. When a plurality of different pesticides are used as the wall forming material in a hollow core structure, the different pesticides can be combined in desired ratios for combination with one or more binders for preparing granules of hollow core structures as described above. Thus, the resulting pesticide granules will have a wall surrounding a hollow core wherein the wall comprises the particles with the different pesticides in the designed ratio.

Pesticide compositions may be applied to a site of use by a variety of methods. Solid pesticide compositions may be applied directly to soil or other surface for release of the pesticide component(s). Other pesticide compositions, however, may be preferably admixed with a solvent and then sprayed onto the delivery site. The improved dissolution rates provided by utilizing a hollow core format as described herein can be beneficial for such uses. In particular, the solid, hollow core pesticide granules may be provided in bulk and then the desired mass or volume of the solid, hollow core pesticide granules can be added to a tank sprayer or similar apparatus immediately prior to application. Because the hollow core format can significantly lessen the time to dissolution of the material, the hollow core pesticide granules can rapidly dissolve in the tank sprayer or similar apparatus for application at the site of use without significant delay. This can enable formation of lightweight compositions in a solid format that exhibit ease of storage and transportation while still allowing for rapid delivery at the point of use.

Pesticides that require delivery in a liquid format, such as in the case of herbicides that require direct plant contact, or such as in the case of pesticides that are sprayed for application in homes and the like, can be beneficially configured in a solid format by combining the liquid pesticide with a carrier particle prior to incorporating the pesticide-loaded carrier particles as the wall forming material in hollow core granules. The formed, hollow core pesticide granules then can be dissolved in a suitable solvent prior to application. Suitable carrier particles and binder can be chosen to likewise dissolve and be delivered with the pesticide as an inert component or as an additive. For example, particles of a solid pesticide may be used as a carrier for a liquid pesticide. Similarly, one or more pesticides may be used as the wall forming material to prepare the pesticide hollow core granules, and one or more further pesticides may be used as a coating on the formed particles to provide for combined delivery of multiple pesticides in a given granule.

The ability to provide pesticide composition with different release properties can be particularly beneficial. For example, some pesticides may pose a risk if ingested by wildlife or domestic animals, and it can be beneficial for such pesticides to quickly solubilize, degrade, or the like to reduce the time during which non-desired interactions may occur. This can be a problem with many solid forms of pesticides, which can persist in the environment for a significant length of time. Such materials, however, may be provided according to the present disclosure in a hollow core form that exhibits rapid dissolution, degradation, or the like. Hollow core pesticides may also be configured to exhibit specific properties that make them highly useful in various environments. For example, hollow core granules may be prepared to exhibit buoyance that can make them particularly useful for application in water. Ponds or other freshwater areas may require treatment for various pests, but in can be difficult to provide solid particles in a form that will persist at the surface rather than immediately solubilizing or sinking. Choice of binder and/or inclusion of additives in the wall forming material can be effective to render the overall granule buoyant for a sufficient time that the pesticide agent can be released at the surface of the water. For example, hydrophobic binders may be utilized for this purpose, and additives, such as cellulosics (e.g., cellulosic aerogels), straw (or similar, buoyant plant materials), and various clays may be utilized as additives and/or carriers for pesticides in the wall forming material of the hollow core granules to render the granules buoyant. Controlled release options as already discussed herein may likewise be applied to ensure that the pesticide material is released at the appropriate time after application to the site of treatment.

In example embodiments, hollow core pesticide granules can be prepared with a variety of wall forming materials and a variety of binders. Clays (e.g., bentonite) can be particularly useful as wall forming materials in pesticide granules along with any specific, solid pesticide materials that may be included in the granules. Likewise, clays can be useful as carriers of one or more pesticides that may be typically provided in a liquid form. Suitable binders can include hydrophilic materials such as PEGs of various molecular weights (e.g., PEG 8000, PEG 12000, and/or PEG 35000), or similar materials that will readily solubilize for release of the pesticide materials held in the granules. PEGs and similar binders particularly may have one or more pesticides admixed therewith so that the binder functions as a vehicle for at least part of the pesticide material(s), and the clay particles or similar materials may be utilized as an essentially inert wall former.

In some embodiments, products formed as hollow core structures as described herein may be provided in unit dose forms. As already described above, a wide variety of materials may be used as wall forming materials for hollow core granules, and the resulting product will be a plurality of hollow core granules that can be provided in any mass or volume desired. In some embodiments, however, it may be beneficial for a defined mass or volume of hollow core granules to be provided in combination to achieve a desired dosage. For example, in the field of detergents/cleaners, it may be desirable to provide a defined mass or volume of a laundry detergent composition as a convenient, pre-dosed amount for a single load of laundry. Similar benefits may be achieved in other fields, such as nutritional supplements, fertilizers, pesticides, and even further areas where it may be more convenient for a consumer to have a pre-dosed amount of the hollow core granule products as an alternative to measuring out a desired dose of the individual granules. Any one or more hollow core granule products according to the present disclosure thus may be provided in a unit dose format in addition to or as an alternative to a mass supply of individual granules.

It is known, for example, to provide granular detergent compositions as wells as pastes, gels, slurries, and the like in water-soluble film pouches, which may be referred to as pods. The present compositions provided as hollow core granules likewise may be provided in such unit dose forms wherein a mass of the hollow core granules is provided in a pouch of defined weight and/or volume. Suitable technologies for providing hollow core granules as described in unit dose forms are described, for example, in U.S. Pat. No. 8,669,220 to Huber et al.; U.S. Pat. App. Pub. No. 2002/0033004 to Edwards et al.; U.S. Pat. App. Pub. No. 2007/0157572 to Oehms et al. U.S. Pat. App. Pub. No. 2012/0097193 to Rossetto et al.; U.S. Pat. Nos. 4,973,416; 7,915,213 to Adamy et al.; and U.S. Pat. App. Pub. No. 2006/0281658 to Kellar et al.; all of the foregoing documents being incorporated herein by reference. In an example embodiment, hollow core granules providing a defined product (e.g., laundry detergent, dishwashing detergent, fertilizers, pesticides, etc.) may be enclosed in poly (vinyl alcohol) film using, for example, a simple Uline heat sealer, thereby forming unit dose pods. Any suitable, water-soluble film may be used, and any suitable sealing technology may be utilized to form the pods in any desired mass/volume suitable to provide a defined quantity of the product for a desired end use. Unit doses may likewise be provided in other forms, such as fabric pouches, which may be formed of dissolvable fibers or non-dissolvable fibers.

Other types of unit dose forms are also encompassed by the present disclosure. For example, a unit dose may encompass a mass of solids that are compressed with one or more binding agents. Such unit dose forms may include a content of hollow core granules as described herein for a particular end use. For example, products such as nutritional supplements, chewable teeth cleaning compositions, or other products where it would be undesirable for the product to be enclosed in a film may be provided in such format. In particular embodiments, a desired mass/volume of hollow core granules may be combined with a binding agent that will allow a plurality of the hollow core granules to be retained together as a unit dose. For example, gums (e.g., guar gums or xanthan gums), cellulosic materials, starch materials, and/or water soluble adhesives may be used to create such blocks, tablets, pills, caplets, prills, or other form factors through agglomeration of a plurality of hollow core granules into a single, unit dose form. These blocks or the like formed from the hollow core structures can be significantly lighter and/or significantly faster dissolving in comparison to known unit dose powders that do not include the present hollow core structures.

In light of the improved absorption properties discussed previously herein, the hollow core granules can be particularly effective in formation of one or more products where absorption and/or adsorption of a gas and/or liquid is desired. In some embodiments, hollow core granules may be configured for use in absorption and/or adsorption of one or more air pollutants. Many materials are classified, such as by the US Environmental Protection Agency, as air pollutants. Non-limiting examples of such materials are: carbon monoxide, lead, nitrogen oxides, ozone, particulate matter, sulfur dioxide, acrolein, asbestos, benzene, carbon disulfide, creosote, fuel oils/kerosene, polycyclic aromatic hydrocarbons, synthetic vitreous fibers, total petroleum hydrocarbons, and the like. Many materials are likewise known as being effective at absorbing, adsorbing, or otherwise binding with one or more of these or other types of air pollutants. Such materials can be used as wall forming materials in preparing hollow core granules according to the present disclosure. Granules with these air pollution capturing components can be deployed in various form factors for interaction with ambient air for capture of one or more air pollutants. Likewise, the granules maybe embedded in articles, such as air filters, industrial pollution capture items (e.g., power plant gas filters and exhaust cleaners), and the like that can be deployed in suitable locations for capture of air pollutants and then disposal. Such granules likewise may be utilized in personal items, such as breathing masks, to remove air pollutants for personal use. As a non-limiting example, activated carbon, zeolites, and other porous materials are known to be effective in capture of various pollutants, and such materials may be incorporated into hollow core granules as wall forming materials and thus provide granules effective at capturing one or more pollutants as well as capturing odors, and the like.

In some embodiments, hollow core granules may be configured for use in absorption or other capture of liquids. Because the hollow core granules can be configured with excellent liquid absorption properties, and because the granules may be configured to preferentially uptake aqueous or hydrophobic liquids, the hollow core granules can be used in a variety of manners for removal of liquids and/or remediation of liquid spill sites. In particular embodiments, the hollow core granules can be configured to absorb one or more types of liquids without solubilization of the granule itself. In this matter, liquids can be bound by the granules, which in turn may be removed in a substantially coherent mass and/or as individual granules that substantially retain the granular structure. This can be particularly useful in removal of organic spills (e.g., oil) in marine or other water environments. The hollow core granules can be configured to substantially float on the water surface (e.g., exhibiting buoyancy as previously discussed herein) where interaction with spilled organics can be maximized. Again, the hollow core granules can be configured to retain their granular structure and/or agglomerate into masses that are relatively easy to remove once the binding activity has been completed. This can extend to preparation of unitary items, such as spill sleeves and the like, wherein the hollow core granules may be retained within a fabric, mesh, or otherwise porous item that will prevent dispersion of the individual granules across the water surface while still allowing influx of the organic material to be retained. In some embodiments, the granules likewise can incorporate components that provide functions in addition to absorption and/or adsorption. For example, biological components are known to be useful to degrade organics or render certain materials less viscous so as to improve the ability of the material to be absorbed.

The presently disclosed, hollow core granules can be easily formulated for the desired end use through choice of wall forming material and/or binder material, and this is shown in the appended examples. For example, it has been shown herein that utilization of a hydrophobic binder provides for hollow core granules that will float on the surface of water (i.e., exhibiting buoyancy). On the other hand, utilization of a hydrophilic binder, such as PEG, can provide hollow core granules that are hydrophilic and that will readily sink in water to quickly dissolve. Moreover, it has been shown herein that the use of the same wall forming material (e.g., bentonite), when used with a hydrophilic binder, such as PEG, or when used with a hydrophobic binder, such as paraffin, can result in two different types of hollow core granules that will behave similarly in both aqueous environments and non-aqueous environments. Thus, choice of a highly stable wall forming material, such as bentonite, can dictate granule properties irrespective of choice of binder. Still further, granules can be additionally modified through use of coating materials to even further adjust the properties. As an example, combination of a hydrophilic binder (e.g., PEG) with a highly stable wall forming material, such as bentonite, can result in a generally hydrophilic granule, but the granule can be modified through formation of a hydrophobic coating layer to render the otherwise hydrophilic granules buoyant and thus allow for floating in aquatic settings to allow the granules to carry out a designed function where buoyancy is desired. Thus, for example, hollow core granules formed of bentonite and PEG but coated with a hydrophobic layer, such as paraffin, can be rendered useful for oil spill cleanup in marine settings.

### EXPERIMENTAL

The present disclosure is more fully illustrated by the following examples, which are set forth to illustrate certain embodiments of the present disclosure and are not to be construed as limiting thereof.

Experimental Methods

A variety of samples of hollow core granules were prepared utilizing a fluidized bed drier. For each set of samples, 5 grams of the chosen binder in particulate form with the chosen particle size were loaded to the fluidized bed drier with 250 grams of the chosen wall forming material in particulate form with the chosen particle size. This provided an excess of the amount of wall forming material necessary to be bound together for formation of the hollow core granules. After processing, the formed granules were unloaded from the fluidized bed drier (leaving behind any remaining, unbound, wall forming material) and weighed. Since all of the loaded binder was used in the granule formation, but since not all the particles of the wall forming material were used, binder concentration of the formed granules was calculated as the total weight of the formed granules in grams divided by 5 grams (the initially loaded weight of the binder). Granule formation using 5 grams of binder and 250 grams of wall forming material (in these examples, sodium bicarbonate or bentonite clay) typically was effective to prepare approximately 50 grams of the hollow core granules.

Granule and Cavity Sizes

To measure average size of the formed granules, average size of the internal cavity (i.e., the hollow core), and average wall thicknesses, 20 randomly selected granules from each test set were cut in half using an Exacto knife, and the half that was visually observed to better retain the original shape was measured microscopically using a microscopic ruler. For granules exhibiting a substantially elongated shape, three measurements were taken for each dimension, and an average of the sum of the three measurements was recorded.

Granule Density

To measure granule density, a cup with a known volume of 33.5 mL was filled with granules and weighed. The obtained weight was divided by the known volume to establish granule density, and a bulk density was obtained as an average of 5 measurements for a given sample of granules.

Granule Strength

Strength of the granules was measured as a maximum force needed to crush the granule. Testing was carried out using the Model 5 ST Benchtop Tester (5 kN/lk lbf) from Tinius Olsen. The machine probe was set to move with a speed of 100 mm/min. Each measurement was repeated with ten randomly selected granules from the respective set of granules. The resistant force was recorded, and the maximum peak force was taken as a strength value. Ten measurements were averaged to provide the final number for the granule strength in the given batch.

Water Absorption

To measure absorption of water by the granules, 1 gram of water was dropped on a prepared layer of granules having an average thickness of 1 cm. The combination was allowed to sit for 5 minutes to form a clump prior to weighing. Percentage of water absorption was calculated according to the following formula: [(weight of clump−1 g)/1 g]×100%.

Oil Absorption

To measure absorption of oil by the granules, 8 grams of granules were placed in oil in a sieve for 5 minutes. The combination was allowed to sit for 5 minutes to drain excess oil and allow any remaining, free oil to be absorbed by the filtering paper. The granules were then weight, and the percentage of oil absorption was calculated according to the following formula: [(weight of wet granules−8 g)/8 g]×100%.

Stability in Water

To evaluate water stability, 3 grams of granules were loaded in a beaker filled with 0.03 liters of water at room temperature (approximately 22° C.). The granules were monitored to determine when the granules begin to disintegrate. The time between loading the granules into the water-filled beaker and the moment when they disintegrate was measured and reported as time for stability in water.

Stability in Oil

To evaluate oil stability, 3 grams of granules were loaded in a beaker filled with 0.03 liters of Lukoil Standard 10 W-40 multigrade mineral engine oil (API SF/CC) at room temperature (approximately 22° C.). The granules were monitored to determine when the granules begin to disintegrate. The time between loading the granules into the oil-filled beaker and the moment when they disintegrate and appear as a sediment was measured and reported as time for stability in oil.

Buoyancy

To evaluate ability of the granules to remain floating in water, 3 grams of granules were loaded in a beaker filled with 0.15 liters of water at room temperature (approximately 22° C.). The combination was immediately evaluated, and granules were noted as being buoyant in water in all cases where a majority of the granules were observed to float.

Solubilization Time in Stirred Water

To further evaluate aqueous solubilization, 10 grams of granules were loaded in a beaker filled with 1.4 liters of deionized water at room temperature (approximately 22° C.). An impeller agitator was included in the beaker set at 500 rpm. The time between loading the granules and the moment when they disappear (i.e., the solution became substantially clear indicating substantial dissolution) was measured and reported as solubilization time. The measurements were performed only for samples where the wall forming materials was a water soluble solid (i.e., sodium bicarbonate).

### Example 1: Sodium Bicarbonate+PEG

Granules were prepared using 5 grams of PEG 8000 (1.2 mm-1.6 mm nominal size) as binder particles or crystals, which was loaded in the fluidized bed drier with 250 grams of sodium bicarbonate (0.100 mm-0.400 mm nominal size). The fluidized bed dryer was run at 65° C. to prepare five batches with different residence times at maximum temperature (5 min, 10 min, 15 min, 20 min, or 30 min). Respective batches were cooled to 30° C. prior to discharging the formed granules. An image of one of the hollow core granules after being cut is shown in FIG. 24.

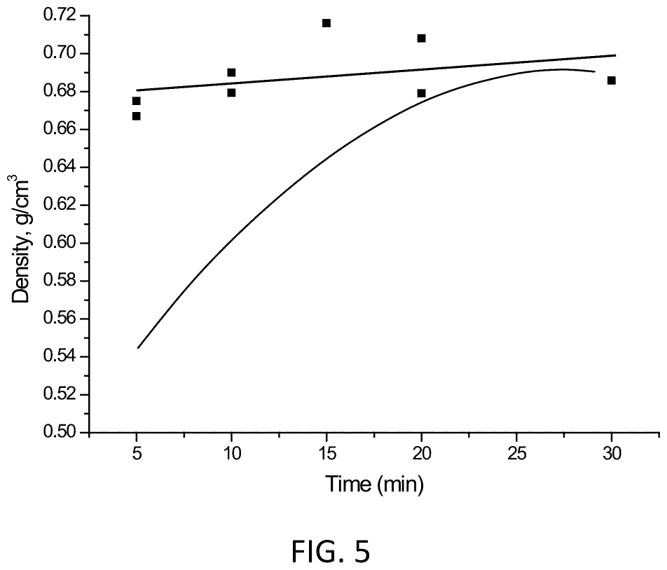

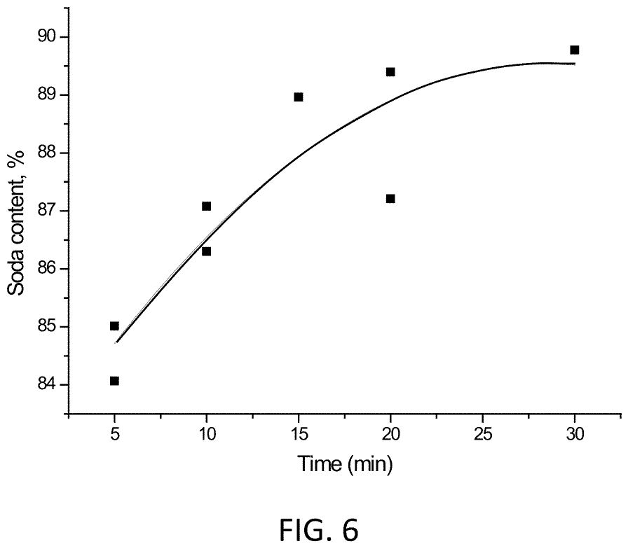

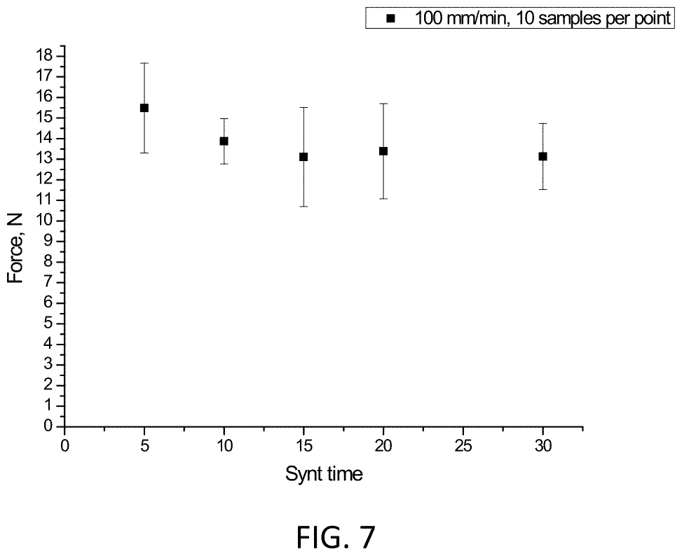

Bulk density of the granules as a function of the processing time in the fluidized bed drier was found to substantially increase with increased residence time, and the measured values are shown in FIG. 5. Residence time was also found to be a factor in the total amount of the sodium bicarbonate particles present in the formed granules with the total content increasing with processing time but appearing to reach a plateau when the binder was fully utilized, and this is shown in FIG. 6. Granule strength was found to remain approximately unchanged with only slight reductions as processing time increased (see FIG. 7). A similar pattern was seen in relation to particle attrition (see FIG. 8A through FIG. 8E).

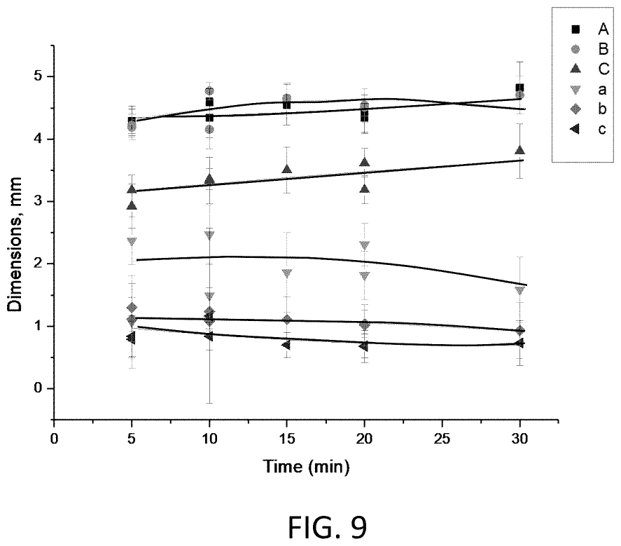

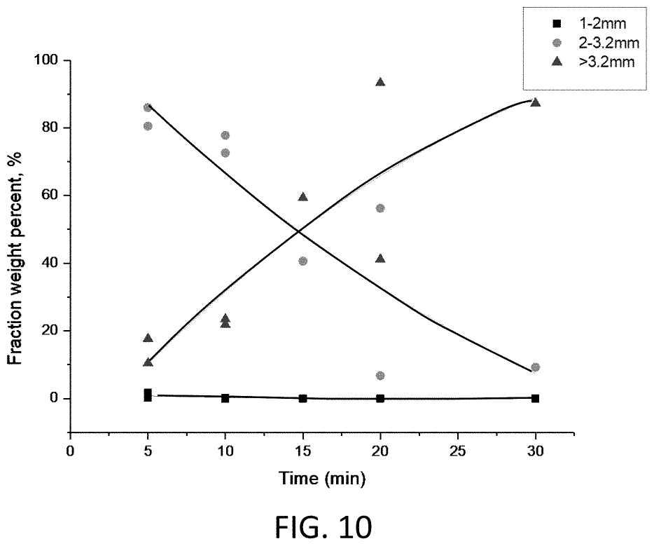

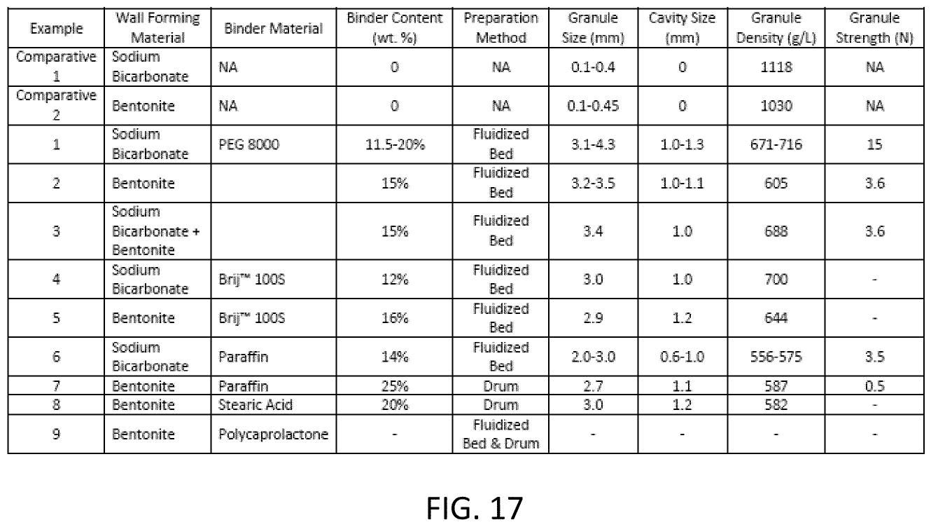

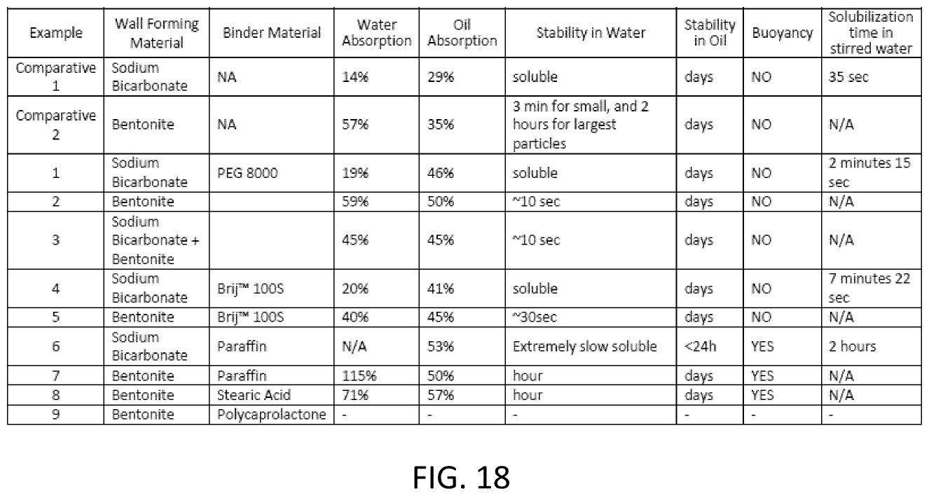

The size of the cavity in the formed granule was determined to be strongly influenced by the initial size of the binder particle. The shell thickness was approximately the same as diameter of the cavity, as shown in FIG. 9, where A, B, and C are the outer dimensions of the granules and a, b, and c are the dimensions of the cavity. Therefore, the diameter of the cavity was found to be approximately ⅓ of the outer diameter of the granule. Cavity volume was thus calculated to be approximately 3-4% of the total volume of the granule. The fractional composition of the typical batch of hollow core granules is shown in FIG. 10, and this was found to again be dependent on residence time in the fluidized bed. The fractional composition was evaluated in compliance with ASTM E-11 using three sieves (1 mm, 2 mm, and 3.2 mm nominal sizes). It was generally found that larger granule sizes were obtained with a longer residence time in the fluidized bed, allowing longer periods for particles of the wall forming material to agglomerate with the binder crystals (see FIG. 10). Total granule properties are shown in the tables of FIG. 17 and FIG. 18.

### Example 2: Bentonite+PEG

Granules were prepared using 5 grams of PEG 8000 (1.2 mm-1.6 mm nominal size) as binder particles or crystals, which was loaded in the fluidized bed drier with 250 grams of bentonite (0.100 mm-0.400 mm nominal size). The fluidized bed dryer was run at 65° C. to prepare two batches with different residence times at maximum temperature (15 min and 30 min). Respective batches were cooled to 30° C. prior to discharging the formed granules. An image of one of the hollow core granules after being cut is shown in FIG. 25.

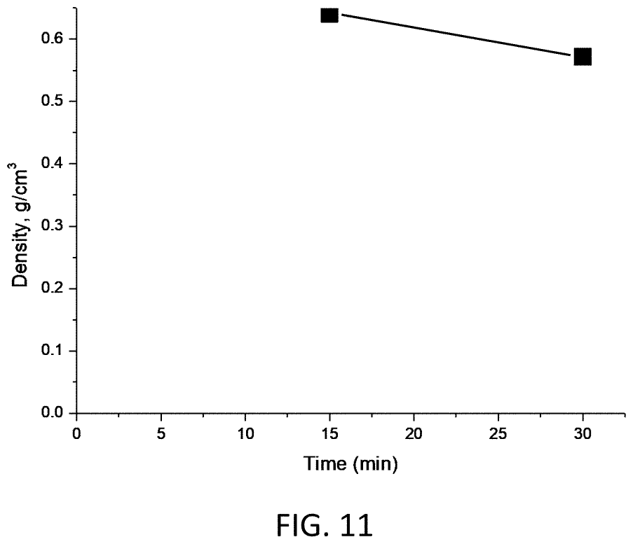

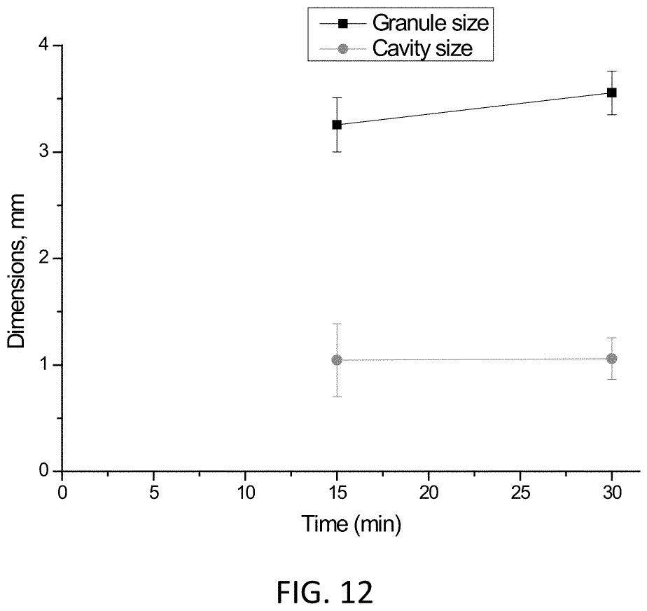

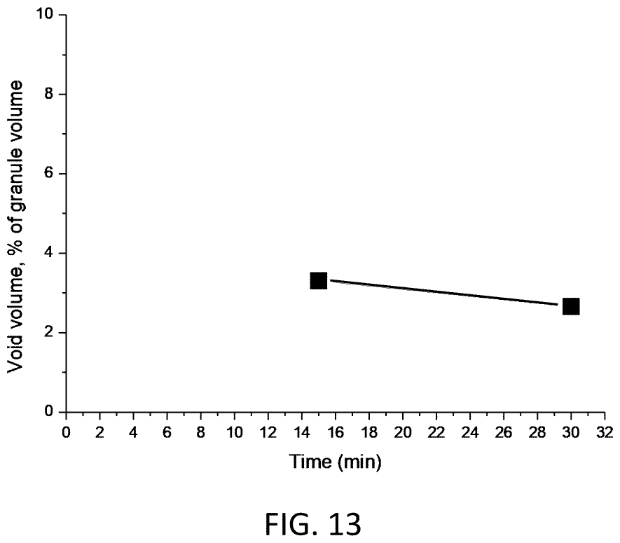

Bulk density of the granules as a function of the processing time in the fluidized bed drier was found to substantially decrease with increased residence time, and the measured values are shown in FIG. 11. Granule strength was found to be less than the measured strength of the granules of Example 1 (3.6 N for the present granules versus 15 N for the granules of Example 1). The shell thickness of the bentonite hollow core granules was approximately equal to the diameter of the cavity (see FIG. 12). Thus, the diameter of the cavity was found to be approximately ⅓ of the total diameter of the granule. Therefore, the volume of the cavity was found to be about 3-4% of the total volume of the granule (see FIG. 13). Total granule properties are shown in the tables of FIG. 17 and FIG. 18.

### Example 3: Sodium Bicarbonate+Bentonite+PEG

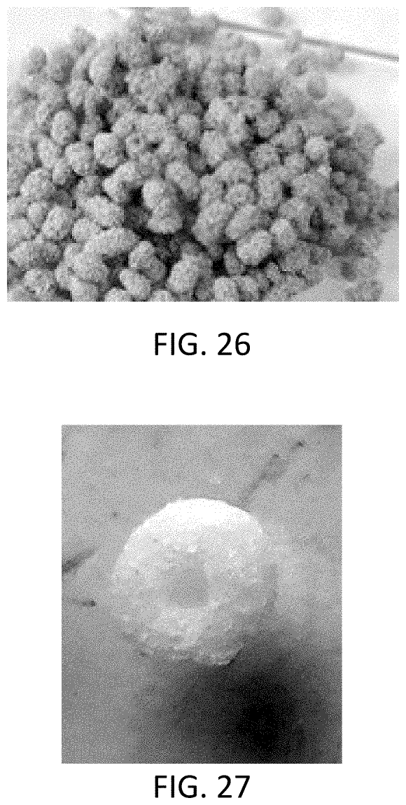

Granules were prepared using 5 grams of PEG 8000 (1.2 mm-1.6 mm nominal size) as binder particles or crystals, which was loaded in the fluidized bed drier with 235 grams of bentonite (0.100 mm-0.400 mm nominal size) and 235 grams of sodium bicarbonate (0.100 mm-0.400 mm nominal size). The fluidized bed dryer was run at 65° C. for 15 minutes prior to cooling to 30° C. before discharging the formed granules. The resulting granules had an average granule size that was similar to the bentonite granules of Example 2. The density of the resulting granules was approximately midway between the density of the sodium bicarbonate granules of Example 1 and the bentonite granules of Example 2. Strength of the granules was similar to the strength of the bentonite granules of Example 2. Total granule properties are shown in the tables of FIG. 17 and FIG. 18. An image of a plurality of the hollow core granules is shown in FIG. 26.

### Example 4: Sodium Bicarbonate+Brij™ S100

Granules were prepared using 5 grams of Brij™ S100 (1.2 mm-1.6 mm nominal size) as binder particles or crystals, which was loaded in the fluidized bed drier with 250 grams of sodium bicarbonate (0.100 mm-0.400 mm nominal size). The fluidized bed dryer was run at 60° C. Immediately after reaching the maximum temperature, the granules were cooled to 30° C. before discharging the formed granules (i.e., residence time of substantially 0). While the Brij™ S100 has a similar melting temperature as PEG 8000, the resulting liquid is much less viscous than the liquefied PEG. This resulted in a significantly faster processing speed with the liquefied Brij™ S100 penetrating the agglomerated wall forming particles to leave behind the cavity defining the hollow core. The properties of the formed granules were substantially similar to the PEG+Sodium Bicarbonate granules with the exception of the solubility parameters since the Brij™ S100 is somewhat more hydrophobic than the PEG. Total granule properties are shown in the tables of FIG. 17 and FIG. 18. An image of one of the hollow core granules after being cut is shown in FIG. 27.

### Example 5 Bentonite+Brij™ S100

Granules were prepared using 5 grams of Brij™ S100 (1.2 mm-1.6 mm nominal size) as binder particles or crystals, which was loaded in the fluidized bed drier with 250 grams of bentonite (0.100 mm-0.400 mm nominal size). The fluidized bed dryer was run at 60° C. Immediately after reaching the maximum temperature, the granules were cooled to 30° C. before discharging the formed granules (i.e., residence time of substantially 0). The properties of the formed granules were substantially similar to the PEG+Bentonite granules. The Brij™ S100+Bentonite granules exhibited a somewhat higher water stability due to the Brij™ S100 being somewhat more hydrophobic than the PEG. Total granule properties are shown in the tables of FIG. 17 and FIG. 18. An image of one of the hollow core granules after being cut is shown in FIG. 28.

### Example 6: Sodium Bicarbonate+Paraffin

Granules were prepared using 5 grams of paraffin (1.2 mm-1.6 mm nominal size) as binder particles or crystals, which was loaded in the fluidized bed drier with 250 grams of sodium bicarbonate (0.100 mm-0.400 mm nominal size). The fluidized bed dryer was run at 55° C. using a residence time of 0, 5 min, and 10 min at maximum temperature. The granules were cooled to 30° C. before discharging the formed granules. Since paraffin has a lower melting temperature than PEG, granule formation was accordingly faster than observed using PEG as the binder.

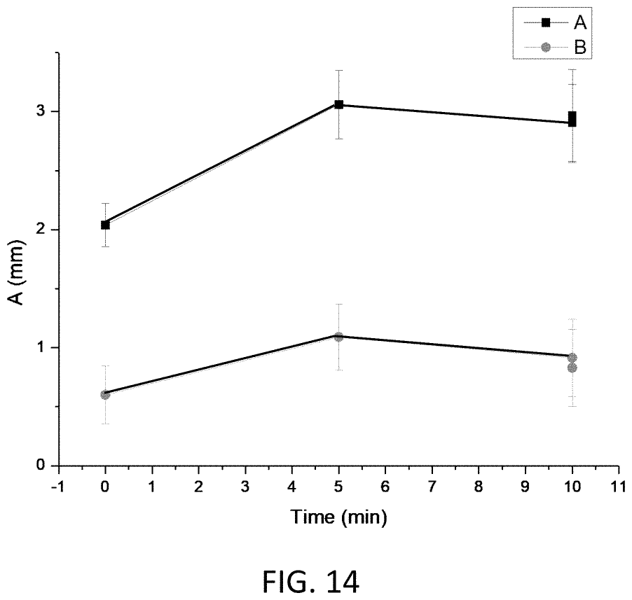

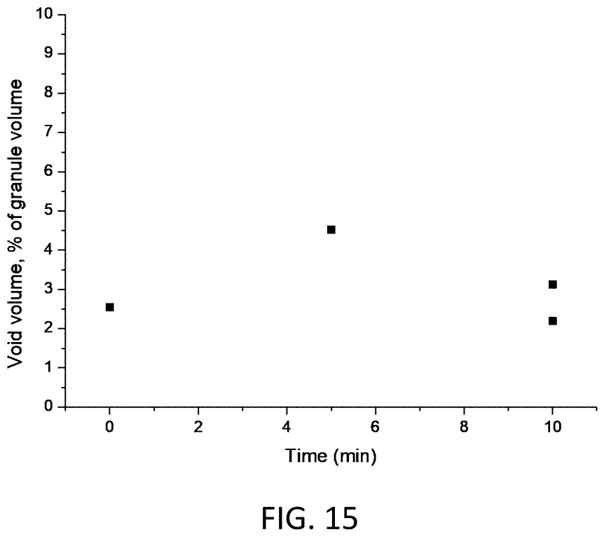

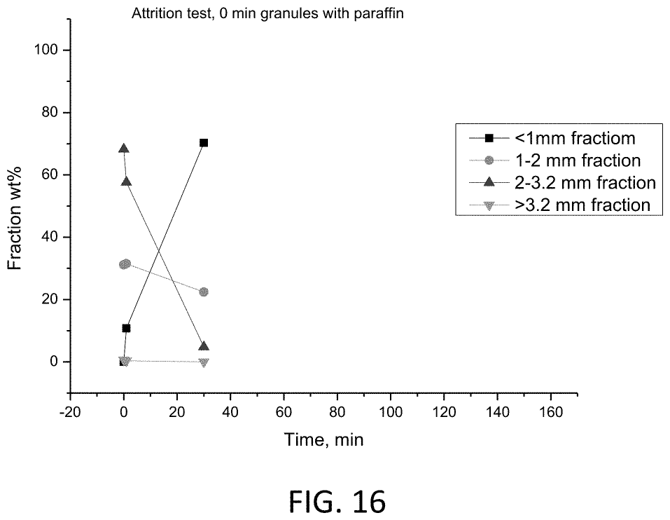

Bulk density of the formed granules was found to be lower than when using sodium bicarbonate wall forming particles with either PEG 8000 or Brij™ S100 as the binder—i.e., 556 to 575 grams per liter for the present granules versus 671 to 716 grams per liter for the granules formed using PEG 8000 or Brij™ S100 as the binder. As seen in FIG. 14, the total granule size and the cavity size was slightly smaller with a 0 min residence time, but increased with additional residence time in the fluidized bed. The volume of cavity was found to account for approximately 2-5% of the total granule volume (see FIG. 15). The granules appeared to be relatively weaker than PEG based granules, which was demonstrated both by attrition testing (see FIG. 16) and crush strength measurement (the present granules having a strength of 3.5 N). Total granule properties are shown in the tables of FIG. 17 and FIG. 18.

### Example 7: Bentonite+Paraffin

Testing indicated that granules formed of bentonite and paraffin lacked sufficient strength to be survive the small amount of shear force encountered during preparation in a fluidized bed method as described above. Without wishing to be bound by theory, it is believed that the hydrophilic nature of the bentonite and the hydrophobic nature of the paraffin was at least partially responsible for sufficient incompatibility to allow for granule formation. Granules, however, we prepared using a drum-like forming process. Granules were prepared using 5 grams of paraffin (1.2 mm-1.6 mm nominal size) as binder particles or crystals. These were manually mixed with approximately 500 grams of bentonite (0.100 mm-0.400 mm nominal size) in a heated pan at 55° C. for 5 minutes. After cooling to 30° C., the formed granules we discharged for evaluation.

The strength of these granules indeed was about 0.5 N (versus about 3.5 N for samples made using PEG or Brig™ S100). Because of hydrophobic nature of paraffin, in the water absorption testing, water did not propagate in the granulated material and rather absorbed in the bentonite particles on the surface, making the result of the measurement artificially high in this method—115%. Most of paraffin-bentonite granules initially floated in water but disintegrated in approximately 1 hour. The granules maintained their integrity in oil for longer than 3 days. Total granule properties are shown in the tables of FIG. 17 and FIG. 18.

### Example 8: Bentonite+Stearic Acid

Granules were again formed using a manual, drum-like process using 5 grams of stearic acid (1.2 mm-1.6 mm nominal size) as binder particles or crystals. These were manually mixed with approximately 500 grams of bentonite (0.100 mm-0.400 mm nominal size) in a heated pan at 75° C. for 5 minutes. After cooling to 30° C., the formed granules we discharged for evaluation. Testing was not attempted using sodium bicarbonate as a wall forming material since sodium bicarbonate is known to decompose in the same temperature range as the melting point of stearic acid (i.e., around 70° C.). The strength and bulk density of the formed granules were similar to the values obtained for the granules made with paraffin and bentonite. In this case, stearic acid behaved as hydrophobic binder very similar to paraffin. Total granule properties are shown in the tables of FIG. 17 and FIG. 18.

### Example 9: Bentonite+Polycaprolactone

Both the fluidized bed method and the manual mixing method described above were utilized in attempting to form hollow core granules using bentonite as a wall forming material and polycaprolactone as the binder, but hollow core granules were not successfully formed. Polycaprolactone is a hydrophobic polymer with a melting temperature of 60° C., and even when using temperatures up to 150° C., the resulting product was a thin coating of bentonite particles surrounding a polycaprolactone, solid core. Without wishing to be bound by theory, it is believed that the viscosity of the liquefied polycaprolactone was too high to allow propagation of the liquid into the bentonite shell. Therefore, in order to successfully form hollow core granules, it was determined that the viscosity of the liquefied binder should be sufficiently low to allow for propagation into the wall of the wall forming particles. It is likewise believed that higher viscosity materials, such as polycaprolactone, but still be utilized when admixed with another binder material and/or a viscosity modifying agent so that the total viscosity of the liquefied binder composition is sufficiently low for hollow granule formation.

### Example 10: Formation of Hollow Core Granules by Hydrogel Method

Hydrogel particles were prepared using a 2.4% by weight solution of agar in water. The solution was heated to approximately 100° C. to dissolve the agar and then cooled down to 60° C. to maintain a suitable solution viscosity for further processing.

Droplets were formed by injecting a stream of the agar solution into a vegetable oil bath chilled to approximately 11° C. The droplets spontaneously formed upon contact with the vegetable oil, and the formed droplets were separated from the oil and washed using a soap solution.

The separated and washed droplets were partially dried in air and then coated with a conditioning composition formed of talcum powder and silicon oil. The conditioned gel droplets were then mixed with bentonite powder until the particles were visually, substantially uniformly coated with the bentonite powder. The coated granules were dried under applied, static heating at a temperature of about 120° C. It was noted that drying time was partially dependent upon the thickness of the granule layer. For example, a substantially single thickness layer dried sufficiently in approximately 1 hour while a layer of granules approximately 1 cm in thickness required approximately 10 hours for desired drying. Although static drying was utilized, forced air may be applied to reduce drying time. Some samples were dried at a temperature of about 160° C., but it was noted that such drying temperature led to undesired shrinkage/deformation of the particles.

### Example 11: Hollow Core Detergent Granules

Hollow core granules were prepared using a powdered detergent composition, and the prepared, hollow core granules were tested against the detergent powder in its native form for changes in properties. The detergent used was a commercially available composition sold under the name Arm and Hammer Crisp Clean Detergent.

To prepare the hollow core granules, particles of Pluriol® E 8000 PEG were loaded into a Sherwood M501 Fluidized Bed Dryer as a binder material with the powdered detergent as the wall forming material, and the materials were processed to form the hollow core detergent granules. Three separate runs were carried out to provide three samples of hollow core detergent granules. Processing was carried out similarly to the preparation methods described above in Examples 1 to 3 to provide “lab scale” granule formation in an amount of about 100 grams of the hollow core granules (test samples 1 to 3 discussed below). Two additional samples were prepared at a manufacturing scale to provide granule formation in amounts of 4 Kg (test sample 4) and 50 Kg (test sample 5) to confirm that observed properties were consistently maintained in large scale production.

Solubility of the hollow core detergent granules was then compared with solubility of the detergent composition in the native form (i.e., “neat”). For each test sample (hollow core granules or neat detergent powder), 10 grams of the sample was dissolved in 1600 mL of room temperature water, and the time in which all granules dissolved was recorded as the solubilization time. A 2000 mL glass beaker was used for this test as well as an IKA Werke EUROSTAR Power-B Overhead Stirrer Mixer with a propeller, mixing at 700 rpm. A stopwatch was used to measure time.

The comparative, neat sample of the detergent powder exhibited a solubilization time of 9 minutes, 32 seconds. The five test samples of the hollow core detergent granules exhibited the following solubilization times: 1) 4 minutes, 30 seconds; 2) 4 minutes, 24 seconds; 3) 4 minutes, 21 seconds; 4) 4 minutes, 37 seconds; and 5) 6 minutes, 11 seconds. As verified by the testing, the presentation of the detergent composition in the hollow core format significant reduced the time to solubilization compared to the detergent composition in its native form. This was surprising since the particle size of the powdered detergent composition was unchanged in the hollow core form versus the native form. Rather, the solubilization time of the detergent composition in the hollow core form was reduced to be even lower than the solubilization time of the binder material. This is believed to be evidence that the overall nature of the hollow core granules provides a more than additive effect in improving solubilization properties, and it is expected that such surprising improvement in physical properties would extend to other functions, such as absorptive properties. Moreover, the significant reduction in solubilization time is believed to support the expectation that presentation of other chemicals, compounds, and compositions in the hollow core format would improve solubilization of such hollow core products and proportionally improve release of components thereof (e.g., cleaners, fertilizers, pesticides, and other materials as discussed herein).

### Example 12: Hollow Core Granule Malodor Testing

Hollow core granules were prepared using a variety of different wall forming materials to evaluate ability to trap odor-causing chemicals and prevent or reduce associated odor. Seven total samples were evaluated: 1) hollow core granules with PEG binder and activated carbon as the wall forming material; 2) hollow core granules with PEG binder and zeolite clinoptilolite as the wall forming material; 3) hollow core granules with PEG binder and sepiolite clay as the wall forming material; 4) hollow core granules with PEG binder and a mixture of sepiolite clay, zeolite clinoptilolite, and activated carbon as the wall forming material; 5) hollow core granules with PEG binder and sodium bicarbonate as the wall forming material; 6) native sodium bicarbonate powder; and 7) native sodium bentonite clay from Bentonite Performance Minerals. Samples 6 and 7 were used as comparatives to compare performance of hollow core granules with materials typically used in products, such as pet litters, for odor reduction. For each of the hollow core granule samples 1 through 5, the respective samples were prepared to comprise about 15% to about 25% by weight of the PEG binder and about 85% to about 75% by weight of the respective wall forming material. SEM images of a zeolite clinoptilolite hollow core granule are provided in FIG. 19A and FIG. 19B. SEM images of an activated carbon hollow core granule are provided in FIG. 20A and FIG. 20B. SEM images of a sodium bicarbonate hollow core granule are provided in FIG. 21A to FIG. 21C.

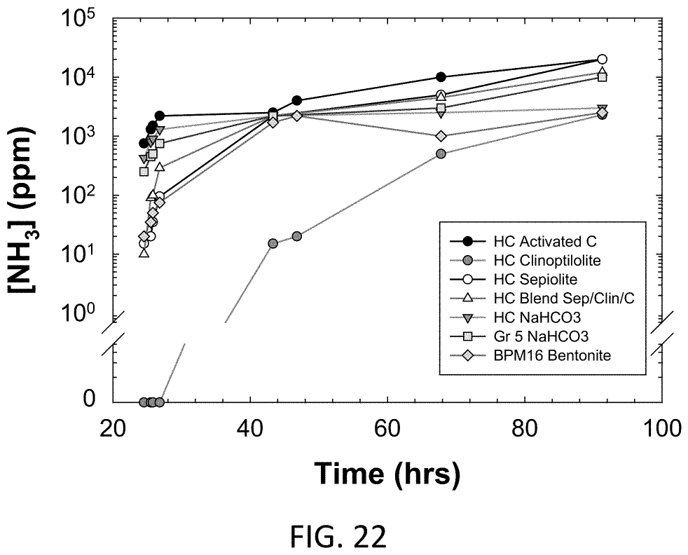

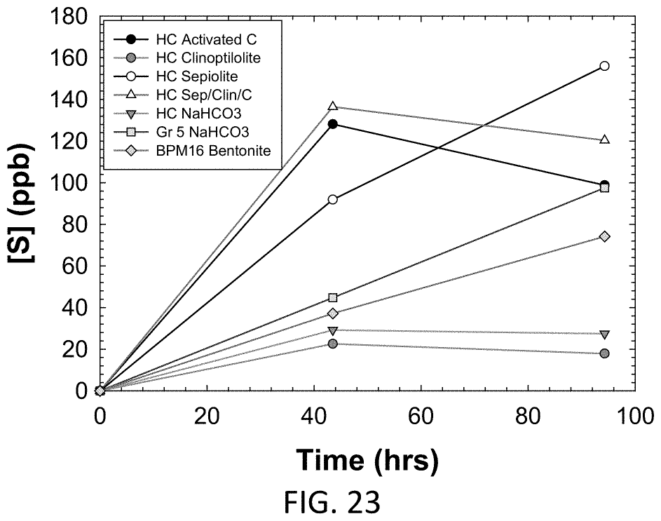

To carry out the malodor testing, approximately 100 grams of the test sample were placed in an Erlenmeyer vacuum flask witted with a side valve and a one-hole stopper. The stopper was fitted with either an additional valve or a Drager ammonia sampling tube (available from Drager, Inc.). To each sample, about 20 mL of a synthetic urine (Felinine, which is an amino acid compound [2-amino-3-propanoic acid] found in cat urine and a precursor via microbial lyase of a putative cat pheromone and thiol [3-mercapto-3-methylbutan-1-ol]). Levels of ammonia were monitored directly from the Drager tube, and a Halimeter (available from Interscan Corporation) was used to periodically measure sulfurous gases. The samples were monitored for approximately 100 hours to measure NH₃ (in ppm) and S (in ppb) with lower concentrations of the respective odor-causing chemicals being indicative of better performance by the test material in capturing the odor-causing chemicals and with higher concentrations of the respective odor-causing chemicals being indicative of lesser performance by the test material in capturing the odor-causing chemicals. Performance for reducing malodor caused by NH₃ is shown in FIG. 22, and performance for reducing malodor caused by S is shown in FIG. 23. For both NH₃ and S gases, the hollow core zeolite clinoptilolite provided the best degree of odor mitigation. As seen in FIG. 23, in a direct comparison of hollow core sodium bicarbonate to native sodium bicarbonate for sulfur odor mitigation, the hollow core form of the material was shown to exhibit superior performance. Specifically, after approximately 100 hours, measured S concentration for the hollow core sodium bicarbonate was approximately 26 ppb while the measured S concentration for native sodium bicarbonate was approximately 100 ppb.

The “about” or “substantially” as used herein can indicate that certain recited values are intended to be read as encompassing the expressly recited value and also values that are relatively close thereto. For example, a value of “about” a certain number or “substantially” as certain value can indicate the specific number or value as well as numbers or values that vary therefrom (+ or −) by 5% or less, 4% or less, 3% or less, 2% or less, or 1% or less. In some embodiments, the values may be defined as being express and, as such, the term “about” or “substantially” (and thus the noted variances) may be excluded from the express value.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A hollow core granule comprising at least one wall substantially surrounding a cavity that is substantially devoid of any solid or liquid so as to define a hollow core, the at least one wall comprising a plurality of individual particles of at least one wall forming material, the plurality of individual particles sufficiently bound together so that the at least one wall is structurally self-sustaining.

2. The hollow core granule of claim 1, wherein the at least one wall forming material is selected from the group consisting of clays, glass, ceramics, aluminas, silicates, zeolites, carbon, metals, salts, absorbents, adsorbents, deodorizers, odor control agents, surfactants, enzymes, bleaches, oxidizers, reducers, gellants, flavors, fragrances, abrasives, fertilizers, insecticides, pesticides, bactericides, herbicides, antimicrobials, anti-sticking agents, fillers, binders, preservatives, optical agents, disinfectants, chelators, molecular binding agents, dyes, coloring agents, colored particles, de-dusting agents, and combinations thereof.

3. The hollow core granule of claim 1, wherein the at least one wall forming material includes a clay.

4. The hollow core granule of claim 1, wherein the at least one wall forming material includes a salt selected from the group consisting of calcium carbonate, sodium chloride, sodium carbonate, sodium bicarbonate, sodium percarbonate, sodium sulfate, sodium carbonate peroxide, potassium chloride, magnesium carbonate, magnesium sulfate, and combinations thereof.

5. The hollow core granule of claim 1, wherein the at least one wall forming material is a fabric care composition.

6. The hollow core granule of claim 5, wherein the fabric care composition is selected from the group consisting of laundry detergents, bleaches, whiteners, brighteners, stain removers, deodorizers, scent boosters, and combinations thereof.

7. The hollow core granule of claim 1, wherein the at least one wall forming material is a pet litter composition.

8. The hollow core granule of claim 1, wherein the at least one wall forming material is an additive for a pet litter composition.

9. The hollow core granule of claim 1, wherein the at least one wall forming material is configured for absorption, adsorption, or other binding of one or more odor causing chemicals in which the hollow core granule comes in contact.

10. The hollow core granule of claim 1, wherein the at least one wall forming material is configured for absorption, adsorption, or other binding of a liquid in which the hollow core granule comes in contact.

11. The hollow core granule of claim 1, wherein the at least one wall forming material is a pH adjusting agent, a fertilizer, a pesticide, or an odor masking agent.

12. The hollow core granule of claim 1, further comprising at least one binder material present in at least a portion of interstitial spaces present between the individual particles of the at least one wall forming material.

13. The hollow core granule of claim 12, wherein the at least one binder is a hydrophilic material.

14. The hollow core granule of claim 13, wherein the at least one binder includes a polyethylene glycol (PEG) material.

15. The hollow core granule of claim 12, wherein the at least one binder is a hydrophobic material.

16. The hollow core granule of claim 15, wherein the at least one binder includes a material selected from the group consisting of wax, paraffin, polycaprolactone, ethylene-vinyl acetate copolymers, polypropylene carbonate, poly(tetramethylene oxide), poly(ethylene adipate), poly(trans-butadiene), thermoplastic polyurethane, stearic acid, and combinations thereof.

17. The hollow core granule of claim 12, wherein the at least one binder comprises about 1% to about 45% by weight, based on the total weight of the hollow core granule.

18. The hollow core granule of claim 1, wherein the hollow core has a diameter that is about 10% to about 80% of a diameter of the hollow core granule.

19. The hollow core granule of claim 1, wherein the hollow core granule is configured so that the cavity that defines the hollow core has a volume that is about 0.1% to about 50% of a volume of the hollow core granule.

20. The hollow core granule of claim 19, wherein the volume of the cavity is about 0.5% to about 10% of the volume of the hollow core granule.

21. The hollow core granule of claim 1, wherein the hollow core granule is buoyant in water.

22. The hollow core granule of claim 1, wherein the at least one wall is an agglomeration of the individual particles of the wall forming material.

23. The hollow core granule of claim 1, wherein the hollow core granule exhibits a time to substantially complete solubilization that is at least 10% faster than a time to substantially complete solubilization of a same weight of the at least one wall forming material alone.

24. A product comprising a plurality of the hollow core granule according to claim 1.

25. The product of claim 24, wherein the product is configured as a cleaning product.

26. The product of claim 25, wherein the cleaning product is a fabric care product.

27. The product of claim 26, wherein the fabric care product is selected from the group consisting of laundry detergents, upholstery cleaners, brighteners, whiteners, stain removers, scent boosters, and combinations thereof.

28. The product of claim 25, wherein the cleaning product is a dishwashing detergent, an abrasive cleaner, or a teeth cleaning product.

29. The product of claim 24, wherein the product is configured as a deodorizer.

30. The product of claim 29, wherein the plurality of the hollow core granule are configured so as to include a material selected from the group consisting of sodium bicarbonate, zeolites, activated carbon, bentonite, and combinations thereof as the at least one wall forming material.

31. The product of claim 24, wherein the product is configured as an animal litter.

32. The product of claim 31, wherein the plurality of the hollow core granule are configured so as to include sodium bicarbonate as the at least one wall forming material.

33. The product of claim 31, wherein the plurality of the hollow core granule are configured so as to include a clay as the at least one wall forming material.

34. The product of claim 33, wherein the clay comprises bentonite.

35. The product of claim 31, wherein plurality of the hollow core granule comprise at least 5% by weight of the animal litter.

36. The product of claim 24, wherein the product is configured as a pet litter additive.

37. The product of claim 24, wherein the product is a fertilizer or a pesticide.

38. A method of preparing hollow core granules, the method comprising:
combining a binder having a melting point of about 40° C. to about 95° C. with a plurality of individual particles of at least one wall forming material that is substantially insoluble in the binder and has a melting point that is greater than the melting point of the binder so as to form a mixture;
heating the mixture to a maximum temperature that is at or above the melting point of the binder and below the melting point of the plurality of the individual particles of at least one wall forming material to form agglomerations of the plurality of the individual particles of at least one wall forming material; and
cooling the agglomerations of the plurality of the individual particles of at least one wall forming material to form the hollow core granules.

39. The method of claim 38, wherein the hollow core granules that are formed comprise at least one wall substantially surrounding a cavity that is substantially devoid of any solid or liquid so as to define a hollow core, the at least one wall comprising the plurality of the individual particles of at least one wall forming material, the plurality of the individual particles sufficiently bound together so that the at least one wall is structurally self-sustaining.

40. The method of claim 39, wherein the binder and the plurality of the individual particles of at least one wall forming material are combined such that an amount of the binder present in the at least one wall of the hollow core granules is about 0.1% to about 50% by weight, based on the total weight of the hollow core granules.

41. The method of claim 38, wherein the process is carried out in a fluidized bed.

42. The method of claim 38, wherein the cooling comprises cooling to a temperature that is below the melting point of the binder.

43. A product comprising hollow core granules prepared according to the method of claim 38.

44. The product of claim 43, wherein the product is selected from the group consisting of laundry detergents, dish detergents, fabric cleaners, fabric deodorizers, abrasive cleaners, teeth cleaning compositions, disinfectants, stain removers, whiteners, brighteners, bleaches, scent boosters, absorbents, adsorbents, deodorizers, odor control products, odor masking products, fertilizers, pesticides, animal litters, and animal litter additives.

45. The product of claim 43, wherein the product is a laundry detergent, and wherein the laundry detergent comprises a mixture of the hollow core granules and one or more further components.

46. The product of claim 43, wherein the product is a laundry detergent, and wherein the plurality of individual particles of at least one wall forming material comprise particles of a laundry detergent composition.

47. The product of claim 43, wherein the product is an animal litter, and wherein the animal litter comprises a mixture of the hollow core granules and one or more further components.

48. The product of claim 43, wherein the product is an animal litter, and wherein the plurality of individual particles of at least one wall forming material comprise particles of a clay or particles of sodium bicarbonate.
